(19) United States
(12) Patent Application Publication
(10) Pub. No.: US 2021/0283111 A1
(43) Pub. Date: Sep. 16, 2021

(54) Methods of Inhibiting Osteoclastogenesis and Osteoclast Activity

(71) Applicant: vTv Therapeutics LLC, High Point, NC (US)

(72) Inventor: Otis Clinton ATTUCKS, Winston-Salem, NC (US)

(21) Appl. No.: 17/335,440

(22) Filed: Jun. 1, 2021

Related U.S. Application Data
(63) Continuation of application No. PCT/US2019/064372, filed on Dec. 4, 2019.
(60) Provisional application No. 62/793,181, filed on Jan. 16, 2019.
(60) Provisional application No. 62/776,540, filed on Dec. 7, 2018.

(51) Int. Cl. A61K 31/428; A61P 19/08

(52) U.S. Cl. CPC A61K 31/428; A61P 19/08

(57) ABSTRACT

The present invention provides methods of inhibiting osteoclastogenesis or inhibiting osteoclast activity using fused imidazole derivative compounds and pharmaceutical compositions. These methods may be used to treat various bone destructive disorders.

### FIELD OF THE INVENTION

The present invention provides methods of inhibiting osteoclastogenesis or osteoclast activity using compounds Formula (I) and pharmaceutical compositions thereof. Methods may be used to treat various degenerative bone diseases.

### BACKGROUND OF THE INVENTION

The strength and integrity of a skeleton depends on an equilibrium between bone resorption by osteoclasts and bone formation by osteoblasts. In certain bone related diseases or as a part of aging, this balance shifts in favor of osteoclasts, and bone resorption exceeds bone formation. As a result of this shortfall, bone density may decrease. In the short term an imbalance may be of little consequence, but if the remodeling cycle is out of balance for a prolonged period, bone turnover can result in major bone loss. As a result, there remains a need for methods and compositions for treating bone degenerative diseases and bone loss.

### SUMMARY OF THE INVENTION

Osteoclastogenesis is a multi-stage process, and each stage presents a potential target for therapeutic intervention. The compounds of Formula (I) herein were disclosed in PCT

Publication No. WO 2011/103018 (“WO '018”) which describes their ability to upregulate expression of HMOX1 in vitro. PCT Publication No. WO 2012/094580 (“WO '580”) describes various compounds and methods of screening for compounds that modulate cellular oxidative stress. Paragraphs [0199] to [0202] of WO '580 describe assays that suggest that fused imidazole derivatives similar to those disclosed in WO '018 may suppress the differentiation of human bone marrow mesenchymal stem cells (BMMSC) into adipocytes.

The present invention is directed to using compounds of Formula (I) to inhibit osteoclastogenesis and/or osteoclast activity. Thus, the present invention is directed to methods of inhibiting osteoclastogenesis using a compound of Formula (I) or a pharmaceutically acceptable salt thereof. The present invention is directed to methods of inhibiting osteoclast activity using a compound of Formula (I) or a pharmaceutically acceptable salt thereof. The present invention is also directed to methods of treating periodontitis or gingivitis using a compound of Formula (I) or a pharmaceutically acceptable salt thereof. The present invention is also directed to methods of treating rheumatoid arthritis using a compound of Formula (I) or a pharmaceutically acceptable salt thereof. The present invention is also directed to methods of inhibiting bone destruction or bone loss using a compound of Formula (I) or a pharmaceutically acceptable salt thereof. The present invention is also directed to methods of maintaining or increasing bone density using a compound of Formula (I) or a pharmaceutically acceptable salt thereof. The present invention is also directed to methods of treating osteoporosis using a compound of Formula (I) or a pharmaceutically acceptable salt thereof. The present invention is also directed to methods of treating osteoarthritis using a compound of Formula (I) or a pharmaceutically acceptable salt thereof. The present invention is also directed to pharmaceutical compositions for use in any of the above methods. The present invention is also directed additional inventions described below.

### BRIEF DESCRIPTION OF THE DRAWINGS

FIG. 1. Cytotoxicity of test compounds against RAW 264.7 cells. Cytotoxicity of various concentrations of various test compounds (HPP-1, -2, -3, and -4) was examined by using alamar blue reagent. Percentage of control is shown. *P<0.05 vs. control.

FIG. 2. Test compounds attenuated RANKL-mediated osteoclastogenesis. Mean number of TRAP-positive multi-nucleated cells differentiated from RAW 264.7 cells. *P<0.05 vs. control. †P<0.05 vs. RANKL alone. NS: not significant difference against the control.

FIGS. 3A-3F. Real time PCR analysis of osteoclast marker gene expression. The expressions of osteoclast marker genes (Atp6v0d2, Trap, Mmp9, Cathepsin K, Dcstamp, and Oscar) in RANKL-stimulated RAW 264.7 cells were examined by real time PCR. Gene expression was calibrated using the Rps18 house-keeping gene, and values indicating the fold-change from control are shown. The data shown are representative of three independent experiments performed in triplicate. *P<0.05 vs. control. †P<0.05 vs. RANKL alone.

FIG. 4. Resorption assay. Resorptive activity was examined using bone resorption assay plate. RANKL (100 ng/mL) stimulation of RAW 264.7 cells induced resorption areas on the substrate. Percentage resorbed area is shown. *P<0.05 vs. RANKL alone. NS: not significant difference against the control.

FIG. 5. Cpd. HPP-4 induced nuclear translocation of Nrf2. Representative images of the western blot analysis of nuclear protein for Nrf2 and histone H3 are shown.

FIGS. 6A-6E. Local HPP-4 injection ameliorated LPS-induced bone destruction in mice. FIGS. 6A-6D—Representative μCT images of control (FIG. 6A), HPP-4 (FIG. 6B), LPS (FIG. 6C), and LPS+HPP-4 treated mice (FIG. 6D) are shown. FIG. 6E—Percentage of resorbed area in the cranial bone. NS: no significant difference between the groups. *P<0.05 vs. control. †P<0.05 between the groups.

### DETAILED DESCRIPTION OF INVENTION

### Definitions

The following definitions are intended to clarify the terms defined. If a particular term used herein is not specifically defined, the term should not be considered to be indefinite. Rather, such undefined terms are to be construed in accordance with their plain and ordinary meaning to a person of ordinary skill in the field(s) of art to which the invention is directed.

As used herein the term “alkyl” refers to a straight or branched chain saturated hydrocarbon having one to ten carbon atoms, which may be optionally substituted, as herein further described, with multiple degrees of substitution being allowed. Examples of “alkyl” as used herein include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl.

The number carbon atoms in an alkyl group is represented by the phrase “C_{x-y} alkyl,” which refers to an alkyl group, as herein defined, containing from x to y, inclusive, carbon atoms. Thus, C₁₋₆ alkyl represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl.

As used herein, the term “alkylene” refers to a straight or branched chain divalent saturated hydrocarbon radical having from one to ten carbon atoms, which may be optionally substituted as herein further described, with multiple degrees of substitution being allowed. Examples of “alkylene” as used herein include, but are not limited to, methylene, ethylene, n-propylene, 1-methylethylene, 2-methylethylene, dimethylmethylene, n-butylene, 1-methyl-n-propylene, and 2-methyl-n-propylene.

The number of carbon atoms in an alkylene group is represented by the phrase “C_{x-y} alkylene,” which refers to an alkylene group, as herein defined, containing from x to y, inclusive, carbon atoms. Similar terminology will apply for other terms and ranges as well. Thus, C₁₋₄ alkylene represents an alkylene chain having from 1 to 4 carbons atoms, and, for example, includes, but is not limited to, methylene, ethylene, n-propylene, 1-methylethylene, 2-methylethylene, dimethylmethylene, n-butylene, 1-methyl-n-propylene, and 2-methyl-n-propylene.

As used herein, the term “cycloalkyl” refers to a saturated, three- to ten-membered, cyclic hydrocarbon ring, which may be optionally substituted as herein further described, with multiple degrees of substitution being allowed. Such “cycloalkyl” groups are monocyclic, bicyclic, or tricyclic. Examples of “cycloalkyl” groups as used herein include, but are not limited to, cyclopropyl, cyclobutyl, cyclopentyl, cyclohexyl, cycloheptyl, norbornyl, and adamantyl.

The number of carbon atoms in a cycloalkyl group will be represented by the phrase “C_{x-y} cycloalkyl,” which refers to a cycloalkyl group, as herein defined, containing from x to y, inclusive, carbon atoms. Similar terminology will apply for other terms and ranges as well. Thus, C₃₋₁₀ cycloalkyl represents a cycloalkyl group having from 3 to 10 carbons as described above, and for example, includes, but is not limited to, cyclopropyl, cyclobutyl, cyclopentyl, cyclohexyl, cycloheptyl, norbornyl, and adamantyl.

As used herein, the term “heterocycle” or “heterocyclyl” refers to an optionally substituted mono- or polycyclic saturated ring system containing one or more heteroatoms. Such “hetercycle” or “heterocyclyl” groups may be optionally substituted as herein further described, with multiple degrees of substitution being allowed. The term “heterocycle” or “heterocyclyl,” as used herein, does not include ring systems that contain one or more aromatic rings. Examples of heteroatoms include nitrogen, oxygen, or sulfur atoms, including N-oxides, sulfur oxides, and sulfur dioxides. Typically, the ring is three- to twelve-membered. Such rings may be optionally fused to one or more of another heterocyclic ring(s) or cycloalkyl ring(s). Examples of “heterocyclic” groups, as used herein include, but are not limited to, tetrahydrofuran, tetrahydropyran, 1,4-dioxane, 1,3-dioxane, piperidine, pyrrolidine, morpholine, tetrahydrothiopyran, and tetrahydrothiophene, where attachment can occur at any point on said rings, as long as attachment is chemically feasible. Thus, for example, “morpholine” refers to morpholin-2-yl, morpholin-3-yl, and morpholin-4-yl.

As used herein, when “heterocycle” or “heterocyclyl” is recited as a possible substituent, the “heterocycle” or “heterocyclyl” group can attach through either a carbon atom or any heteroatom, to the extent that attachment at that point is chemically feasible. For example, “heterocyclyl” would include pyrrolidin-1-yl, pyrrolidin-2-yl, and pyrrolidin-3-yl. When “heterocycle” or “heterocyclyl” groups contain a nitrogen atom in the ring, attachment through the nitrogen atom can alternatively be indicated by using an “-ino” suffix with the ring name. For example, pyrrolidino refers to pyrrolidin-1-yl.

As used herein the term “halogen” refers to fluorine, chlorine, bromine, or iodine.

As used herein, the term “oxo” refers to a >C═O substituent. When an oxo substituent occurs on an otherwise saturated group, such as with an oxo-substituted cycloalkyl group (e.g., 3-oxo-cyclobutyl), the substituted group is still intended to be a saturated group.

As used herein, the term “heteroaryl” refers to a five- to fourteen-membered optionally substituted mono- or polycyclic ring system, which contains at least one aromatic ring and also contains one or more heteroatoms. Such “heteroaryl” groups may be optionally substituted as herein further described, with multiple degrees of substitution being allowed. In a polycyclic “heteroaryl” group that contains at least one aromatic ring and at least one non-aromatic ring, the aromatic ring(s) need not contain a heteroatom. Thus, for example, “heteroaryl,” as used herein, would include indolinyl. Further, the point of attachment may be to any ring within the ring system without regard to whether the ring containing the attachment point is aromatic or contains a heteroatom. Thus, for example, “heteroaryl,” as used herein, would include indolin-1-yl, indolin-3-yl, and indolin-5-yl. Examples of heteroatoms include nitrogen, oxygen, or sulfur atoms, including N-oxides, sulfur oxides, and sulfur dioxides, where feasible. Examples of “heteraryl” groups, as used herein include, but are not limited to, furyl, thiophenyl, pyrrolyl, oxazolyl, thiazolyl, imidazolyl, isoxazolyl, isothiazolyl, 1,2,4-triazolyl, pyrazolyl, pyridinyl, pyridazinyl, pyrimidinyl, indolyl, isoindolyl, benzo[b]thiophenyl, benzimidazolyl, benzothiazolyl, pteridinyl, and phenazinyl, where attachment can occur at any point on said rings, as long as attachment is chemically feasible. Thus, for example, “thiazolyl” refers to thiazol-2-yl, thiazol-4-yl, and thiaz-5-yl.

As used herein, when “heteroaryl” is recited as a possible substituent, the “heteroaryl” group can attach through either a carbon atom or any heteroatom, to the extent that attachment at that point is chemically feasible.

As used herein, the term “heterocyclylene” refers to an optionally substituted bivalent heterocyclyl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to,

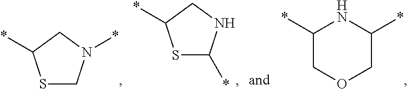

where the asterisks indicate points of attachment.

As used herein, the term “heteroarylene” refers to an optionally substituted bivalent heteroaryl group (as defined above). The points of attachment may be to the same ring atom or to different ring atoms, as long as attachment is chemically feasible. The two points of attachment can each independently be to either a carbon atom or a heteroatom, as long as attachment is chemically feasible. Examples include, but are not limited to,

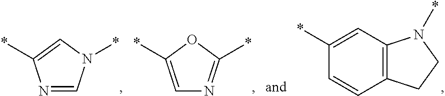

where the asterisks indicate points of attachment.

Various other chemical terms or abbreviations have their standard meaning to the skilled artisan. For example: “hydroxyl” refers to —OH; “methoxy” refers to —OCH₃; “cyano” refers to —CN; “amino” refers to —NH₂; “methylamino” refers to —NHCH₃; “sulfonyl” refers to —SO₂—; “carbonyl” refers to —C(O)—; “carboxy” or “carboxyl” refer to —CO₂H, and the like. Further, when a name recited multiple moieties, e.g., “methylaminocarbonyl-methyl”, an earlier-recited moiety is further from the point of attachment than any later-recited moieties. Thus, a term such as “methylaminocarbonylmethyl” refers to —CH₂—C(O)—NH—CH₃.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a subject. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

When any variable occurs more than one time in any one constituent (e.g., R^{d}), or multiple constituents, its definition on each occurrence is independent of its definition on every other occurrence.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

As used herein, the term “optionally” means that the subsequently described event(s) may or may not occur.

As used herein, “administer” or “administering” means to introduce, such as to introduce to a subject a compound or composition. The term is not limited to any specific mode of delivery, and can include, for example, intravenous delivery, transdermal delivery, oral delivery, nasal delivery, and rectal delivery. Oral delivery may include administering directly to affected areas in a subject's mouth, such as administering to gum, teeth, or other locations in a subject's oral cavity. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of delaying the progress of a disease or condition, controlling a disease or condition, delaying the onset of a disease or condition, ameliorating one or more symptoms characteristic of a disease or condition, or delaying the recurrence of a disease or condition or characteristic symptoms thereof, depending on the nature of a disease or condition and its characteristic symptoms. “Treat” or “treating” or “treatment” may also refers to inhibiting the disease, either physically, (e.g., stabilization of a discernible symptom), physiologically, (e.g., stabilization of a physical parameter), or both, and to inhibiting at least one physical parameter that may or may not be discernible to the subject. In certain embodiments, “treat” or “treating” or “treatment” refers to delaying the onset of the disease or at least one or more symptoms thereof in a subject which may be exposed to or predisposed to a disease even though that subject does not yet experience or display symptoms of the disease.

As used herein, “subject” may refer any mammal such as, but not limited to, humans. In one embodiment, the subject is a human. In another embodiment, the host is a human who exhibits one or more symptoms characteristic of a disease or condition. The term “subject” does not require one to have any particular status with respect to any hospital, clinic, or research facility (e.g., as an admitted patient, a study participant, or the like). In an embodiment, the subject may be “a subject in need thereof.” In some embodiments, the subject may be one that already has osteoporosis or another bone destructive disorder as described herein. In other embodiments, the subject may be one that is at risk of developing osteoporosis or another bone destructive disorder as described herein. For instance, the subject may be one that is elderly or soon to be elderly. In some cases, the subject may be greater than 40, greater than 45, greater than 50, greater than 55, greater than 60, greater than 65, greater than 70, greater than 75, greater than 80, or greater than 85 years old. The subject may be a post-menopausal woman. The subject may also be one with a family history of osteoporosis or another bone destructive disorder. The subject may have certain risk factors for osteoporosis, such as being female, Caucasian, of Asian descent, having a family history of osteoporosis, having a family history of fractures, and/or having a small body frame. In an embodiment, a subject is one who has been diagnosed as having or at risk of having a bone destruction disorder following a determination as described below in the Methods of Diagnosis.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

As used herein, the term “compound of the invention” includes free acids, free bases, and any salts thereof. Thus, phrases such as “compound of embodiment 1” or “compound of claim 1” refer to any free acids, free bases, and any salts thereof that are encompassed by embodiment 1 or claim 1, respectively.

### II. Methods of Treatment

A. Treatment of Bone Disorders

In an embodiment, the present invention is directed to a method of inhibiting osteoclastogenesis comprising administering to a subject a compound of Formula (I) or a pharmaceutically acceptable salt thereof.

In another embodiment, the present invention is directed to a method of inhibiting osteoclast activity comprising administering to a subject a compound of Formula (I) or a pharmaceutically acceptable salt thereof.

In another embodiment, the present invention is directed to a method of treating periodontitis or gingivitis comprising administering to a subject a compound of Formula (I) or a pharmaceutically acceptable salt thereof. In a further embodiment, the method comprises administering an amount of a compound of Formula (I) or a pharmaceutically acceptable salt thereof sufficient to inhibit osteoclastogenesis or osteoclast activity in the subject.

In another embodiment, the present invention is directed to a method of inhibiting bone destruction, inhibiting bone loss, or inhibiting the rate of reduction in bone density comprising administering to a subject a compound of Formula (I) or a pharmaceutically acceptable salt thereof. In a further embodiment, the method comprises administering an amount of a compound of Formula (I) or a pharmaceutically acceptable salt thereof sufficient to inhibit osteoclastogenesis or osteoclast activity in the subject.

In another embodiment, the present invention is directed to a method of maintaining or increasing bone density or bone mineral density comprising administering to a subject a compound of Formula (I) or a pharmaceutically acceptable salt thereof. In an further embodiment, the method comprises administering an amount of a compound of Formula (I) or a pharmaceutically acceptable salt thereof sufficient to inhibit osteoclastogenesis or osteoclast activity in the subject.

In another embodiment, the present invention is directed to a method of treating osteoporosis comprising administering to a subject a compound of Formula (I) or a pharmaceutically acceptable salt thereof. In a further embodiment, the method comprises administering an amount of a compound of Formula (I) or a pharmaceutically acceptable salt thereof sufficient to inhibit osteoclastogenesis or osteoclast activity in the subject.

In another embodiment, the present invention is directed to a method of treating osteoarthritis comprising administering to a subject a compound of Formula (I) or a pharmaceutically acceptable salt thereof. In a further embodiment, the method comprises administering an amount of a compound of Formula (I) or a pharmaceutically acceptable salt thereof sufficient to inhibit osteoclastogenesis or osteoclast activity in the subject.

In another embodiment, the present invention is directed to a method of treating rheumatoid arthritis comprising administering to a subject a compound of Formula (I) or a pharmaceutically acceptable salt thereof. In a further embodiment, the method comprises administering an amount of a compound of Formula (I) or a pharmaceutically acceptable salt thereof sufficient to inhibit osteoclastogenesis or osteoclast activity in the subject.

In any of the preceding embodiments, the methods may further comprise the step of determining whether a subject is at risk for or has a bone related disease by obtaining or having obtained a biological sample from the subject and performing or having performed a bodily fluid test on the biological sample to determine if the subject has biomarkers for a bone related disease.

In any of the preceding embodiments, the methods may further comprise the step of determining whether a subject is at risk for or has a bone related disease by performing or having performed a first skeletal survey as described below on an area of the subject's skeleton to determine if the subject has a bone density level associated with a bone related disease. In an embodiment, a subject is selected for treatment if their T-score is below −1, or between −1 and −2.5, or less than −2.5. In an embodiment, a subject is selected for treatment if their Z-score is below −1, or between −1 and −2.5, or less than −2.5.

In any of the preceding methods, the method may further comprise obtaining or having obtained biological samples over a period of time from the subject and performing or having performed a bodily fluid test on the biological samples to determine whether the level of one or more biochemical resorptive markers are increasing or decreasing, and if the level of one or more biochemical resorptive markers are increasing or not decreasing then administering a greater dose of a compound of Formula (I) or a pharmaceutically acceptable salt thereof. For example, osteoclast function may be measured by a reduction in expression of one or more of osteoclast marker genes such as, but not limited to, Atp6v0d2, Matrix metalloprotein 9, Oscar, Cathepsin K, Dcstamp, and Trap. In some embodiments, a compound of Formula (I) or a pharmaceutically acceptable salt thereof is administered to a subject in need thereof is administered in an amount to decrease the level of one or more of these markers. The period between collection of biological samples may be 1 week, 2 weeks, 3 weeks, 4 weeks, 2 months, 3 months, 6 months, 9 months, or 12 months.

In any of the preceding methods, the method may further comprise performing or having performed a second skeletal survey to determine whether the subject's bone density is changing or has changed from the first skeletal survey, and if the subject's bone density is decreasing or has decreased from the first to the second skeletal survey then administering a greater dose and/or a more frequent dose of a compound of Formula (I) or a pharmaceutically acceptable salt thereof. The period between the first and second skeletal surveys may be 1 week, 2 weeks, 3 weeks, 4 weeks, 2 months, 3 months, 6 months, 9 months, or 12 months. The dose and/or frequency may be increased if the difference in bone density between the first and second survey is greater than 1%, 2%, 3%, 4%, 5%, 6%, 7%, 8%, 9%, or 10%.

A compound of Formula (I) or a pharmaceutically acceptable salt thereof may be administered in a dosage of between 0.1 mg and 15 mg per kg. In another embodiment, where the subject is a human the daily dose may be between 1 mg and 1000 mg. In another embodiment, a compound of Formula (I) or a pharmaceutically acceptable salt thereof is administered in an amount from 10 mg/day to 1000 mg/day, or from 25 mg/day to 800 mg/day, or from 37 mg/day to 750 mg/day, or from 75 mg/day to 700 mg/day, or from 100 mg/day to 600 mg/day, or from 150 mg/day to 500 mg/day, or from 200 mg/day to 400 mg/day, or in an amount of less than 1000 mg/day, or less than 800 mg/day, or less than 750 mg/day, or less than 700 mg/day, or less than 600 mg/day, or less than 500 mg/day, or less than 400 mg/day. In other embodiments, the previous daily periods of administration of an amount of a compound of Formula (I) or a pharmaceutically acceptable salt thereof may be changed to a period of every 6 hours, 12 hours, 48 hours, 72 hours, 96 hours, 1 week, or 2 weeks.

B. Treatments in Combination with Other Active Ingredients

Methods for treatment described above and through this disclosure may also include administering a compound of the invention in combination with or alternation with another active agent.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order. When co-administration involves the separate administration of the first amount of a composition as described herein, and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound. For example, a composition as described herein, and the second therapeutic agent can be administered in any order within about 24 hours of each other, within about 16 hours of each other, within about 8 hours of each other, within about 4 hours of each other, within about 1 hour of each other or within about 30 minutes of each other.

More specifically, a first therapy (e.g., a prophylactic or therapeutic agent such as a composition described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Examples of therapeutic agents that may be combined with a composition of this disclosure, either administered separately or in the same pharmaceutical composition, include, but are not limited to, bisphosphonates (e.g., alendronate, risedronate, ibandronate, zoledronic acid), hormone-related therapy (e.g., estrogen, raloxifene, testosterone), teriparatide, denosumab, calcitonin, parathyroid hormone peptides (e.g., Forteo, Ostabolin-C), osteoblast activating peptide, osteocalcin, and osteogenic growth peptide.

Bisphosphonate drugs are often the first-line treatment. The often prescribed bisphosphonates are sodium alendronate (Fosamax™) orally, risedronate (Actonel™) orally or etidronate (Didronel™) orally, or ibandronate (Boniva™) orally daily or once a month or zolendronate (Zometa™) monthly or yearly intravenously or Pamidronate (Aredia™) monthly or 3-6 monthly intravenously. Oral strontium ranelate is an alternative oral treatment, suggested to stimulate the proliferation of osteoblasts, as well as inhibiting the proliferation of osteoclasts.

Additional examples of such therapeutic agents that may be combined with a composition of this disclosure include, but are not limited to, Nrf2 activators, antioxidants, detoxification agents, and anti-inflammatory agents.

Examples of the Nrf2 activators include sulforaphane, avicins, 15dPGJ₂, xanthohumol, curcumin, carnosol, zerumbone, isothiocyanate, α-lipoic acid, oltipraz (4-methyl-5-[2-pyrazinyl]-1,2-dithiole-3-thione), 1,2-dithiole-3-thione, and 2,3-butyl-4-hydroxyanisole.

Examples of the antioxidants include vitamin C, vitamin E, carotenoids, retinolds, polyphenols, flavonoids, lignan, selenium, butylated hydroxyanisole, ethylene diamine tetra-acetate, calcium disodium, acetylcysteine, probucol, and tempo.

Examples of the detoxification agents include dimethyl caprol, glutathione, acetylcysteine, methionine, sodium hydrogen carbonate, deferoxamine mesylate, calcium disodium edetate, trientine hydrochloride, penicillamine, and pharmaceutical charcoal.

The anti-inflammatory agents include steroidal anti-inflammatory agents and non-steroidal anti-inflammatory agents. Examples of the steroidal anti-inflammatory agents include cortisone acetate, hydrocortisone, paramethasone acetate, prednisolone, prednisolone, methylprednine, dexamethasone, triamcinolone, and betamethasone. Examples of the non-steroidal anti-inflammatory agents include salicylic acid non-steroidal anti-inflammatory agents such as aspirin, difiunisal, aspirin+ascorbic acid, and aspirin dialuminate; aryl acid non-steroidal anti-inflammatory agents such as diclofenac sodium, sulindac, fenbufen, indomethacin, indomethacin farnesyl, acemetacin, proglumetacin maleate, anfenac sodium, nabmeton, mofezolac, and etodorag; fenamic acid non-steroidal anti-inflammatory agents such as mefenamic acid, flufenamic acid aluminum, tolfenamic acid, and floctafenine; propionic acid non-steroidal anti-inflammatory agents such as ibuprofen, flurbiprofen, ketoprofen, naproxen, pranoprofen, fenoprofen calcium, thiaprofen, oxaprozin, loxoprofen sodium, alminoprofen, and zaltoprofen; oxicam non-steroldal anti-inflammatory agents such as piroxicam, ampiroxicam, tenoxicam, lornoxicam, and meloxicam; and basic non-steroidal anti-inflammatory agents such as tiaramide hydrochloride, epirizole, and emorfazone.

### III. Compounds and Compositions for Use in the Methods of Treatment

A. Compounds of the Invention

A compound of Formula (I) has the structure shown below

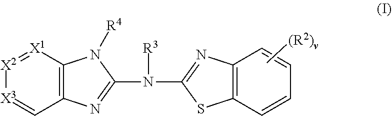

wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h} R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

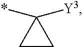

where Y³ is cyclopropyl, —CF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{z};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{d},
   i) —S(O)_{w}—R^{d},
   j) —S(O)₂O—R^{d},
   k) —NR^{d}R^{e},
   l) —C(O)—R^{d},
   m) —C(O)—O—R^{d},
   n) —OC(O)—R^{d},
   o) —C(O)NR^{d}R^{e},
   p) —C(O)-heterocyclyl,
   q) —NR^{d}C(O)R^{e},
   r) —OC(O)NR^{d} R^{e},
   s) —NR^{d} C(O)OR^{d}, or
   t) —NR^{d} C(O)NR^{d} R^{e},
   where the alkyl, cycloalkyl, and heterocyclyl groups are optionally substituted one or more times with substituents independently selected from R^{y};
R^{b} is
   a) -halogen,
   b) —C₁₋₆ alkyl,
   c) —C₃₋₁₀ cycloalkyl,
   d) -heterocyclyl,
   e) -phenyl,
   f) -heteroaryl,
   g) -cyano,
   h) —CF₃,
   i) —OCF₃,
   j) —O—R^{f},
   k) —S(O)_{w}—R^{f},
   l) —S(O)₂O—R^{f},
   m) —NR^{f}R^{g},
   n) —C(O)—R^{f},
   o) —C(O)—O—R^{f},
   p) —OC(O)—R^{f},
   q) —C(O)NR^{f}R^{g},
   r) —C(O)-heterocyclyl,
   s) —NR^{f} C(O)R^{g},
   t) —OC(O)NR^{f} R^{g},
   u) —NR^{f} C(O)OR^{f}, or
   v) —NR^{f} C(O)NR^{f} R^{g},
   where the alkyl, cycloalkyl, heterocyclyl, phenyl, and heteroaryl groups are optionally substituted one or more times with substituents independently selected from R^{z};
R^{c} is
   a) -halogen,
   b) —C₁₋₆ alkyl,
   c) —C₃₋₁₀ cycloalkyl,
   d) -heterocyclyl,
   e) -cyano,
   f) —CFs,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h} R^{k},
   p) —C(O)-heterocyclyl,
   q) —NR^{h} C(O)R^{k},
   r) —OC(O)NR^{h} R^{k},
   s) —NR^{h} C(O)OR^{k},
   t) —NR^{h} C(O)NR^{h} R^{k},
   u) —NR^{h} S(O)_{w}R^{k},
   v) -phenyl,
   w) -heteroaryl, or
   x) —O—(C₁₋₄ alkylene)-O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
   a) -halogen,
   b) —NH₂,
   c) -cyano,
   d) -carboxy,
   e) -hydroxy,
   f) -thiol,
   g) —CF₃,
   h) —OCF₃,
   i) —C(O)—NH₂,
   j) —S(O)₂—NH₂,
   k) oxo,
   l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   z) —S(O)₂—N(C₁₋₆ alkyl)₂, optionally substituted one or more times with substituents selected independently from the group consisting of halogen, —OH, —O—C₁₋₆ alkyl, —NH₂, —NH-Cue alkyl, and —N(C₁₋₆ alkyl)₂,
   aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
   bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
   a) —R^{y}
   b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
R^{z} is
   a) —R^{y}
   b) -phenyl,
   c) -heteroaryl;
   d) —O-phenyl,
   e) —O-heteroaryl,
   f) —C(O)-phenyl,
   g) —C(O)-heteroaryl,
   h) —C(O)—O-phenyl, or
   i) —C(O)—O-heteroaryl;
v is an integer from 0 to 4, and
w is an integer from 0 to 2.
Embodiment 2: A compound according to embodiment 1 wherein
G is hydrogen, —C₁₋₈ alkyl, —C₃₋₁₀ cycloalkyl, —C₁₋₆ alkylene-C₃₋₁₀ cycloaklyl, heterocyclyl, phenyl, heteroaryl, or NR^{h} R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

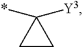

where Y³ is -cyclopropyl, —CF₃, —OCF₃, —OCH₃, —OCH₂CH₃, —F, —Cl, —OH, —O(CH₂)₂—OH′—O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h} R^{k},
   p) —C(O)-heterocyclyl,
   q) —NR^{h} C(O)R^{k},
   r) —OC(O)NR^{h} R^{k},
   s) —NR^{h} C(O)OR^{k},
   t) —NR^{h} C(O)NR^{h} R^{k},
   u) -phenyl,
   v) -heteroaryl, or
   w) —O—(C₁₋₄ alkylene)-O—(C₁₋₄ alkylene)-N(R^{h})C(O)—OR^{k},
   where the alkylene, alkyl, cycloalkyl, heterocyclyl, phenyl, and heteroaryl groups are optionally substituted one or more times with substituents independently selected from R^{x};
R^{h} and R^{k} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{y} is
   a) -halogen,
   b) —NH₂,
   c) -cyano,
   d) -carboxy,
   e) —C₁₋₆ alkyl, optionally substituted one or more times with halogen,
   f) -heterocyclyl, optionally substituted one or more times with halogen,
   g) —C₃₋₁₀ cycloalkyl, optionally substituted one or more times with halogen,
   h) —O—C₁₋₆ alkyl, optionally substituted one or more times with halogen,
   i) —O—C₃₋₁₀ cycloalkyl, optionally substituted one or more times with halogen,
   j) -hydroxy,
   k) -thiol,
   i) —CF₃,
   m) —OCF₃,
   n) —C(O)—C₁₋₆ alkyl, optionally substituted one or more times with halogen,
   o) —C(O)—O—C₁₋₆ alkyl, optionally substituted one or more times with halogen,
   p) —S—C₁₋₆ alkyl, optionally substituted one or more times with halogen, or
   q) —S(O)₂—C₁₋₆ alkyl, optionally substituted one or more times with halogen.
Embodiment 3: A compound according to embodiment 2, wherein
   R³ is hydrogen.
Embodiment 4: A compound according to embodiment 2, wherein
   R³ is methyl.
Embodiment 5: A compound according to any one of embodiments 2 to 4, wherein X¹ is ═N—.
Embodiment 6: A compound according to any one of embodiments 2 to 4, wherein X¹ is ═CH—.
Embodiment 7: A compound according to any one of embodiments 2 to 6, wherein
   v is an integer from 0 to 2.
Embodiment 8: A compound according to any one of embodiments 2 to 6, wherein
   v is 0 or 1.
Embodiment 9: A compound according to any one of embodiments 2 to 6, wherein
   v is 1.
Embodiment 10: A compound according to any one of embodiments 2 to 6, wherein
   v is 1, and R² is attached at either the 5-position or the 6-position of the benzothiazole ring.
Embodiment 11: A compound according to any one of embodiments 2 to 6, wherein
   v is 1, and R² is attached at the 6-position of the benzothiazole ring.
Embodiment 12: A compound according to any one of embodiments 2 to 68, wherein
   v is 2, and one R² is attached at the 6-position of the benzothiazole ring.
Embodiment 13: A compound according to any one of embodiments 2 to 6, wherein
   v is 2, and R² is attached at the 5-position and the 6-position of the benzothiazole ring.
Embodiment 14: A compound according to any one of embodiments 2 to 13, wherein
   R² is -halogen, —C₁₋₆ alkyl, —CF₃, —OCF₃, —O—R^{f}, or —S(O)_{w}—R^{f}, where the alkyl group is optionally substituted one or more times with substituents independently selected from R^{z}.
Embodiment 15: A compound according to any one of embodiments 2 to 13, wherein
   R² is -halogen, -methyl, —CF₃, —OCF₃, —SCF₃, —O-heteroaryl, or —S(O)₂—CH₃.
Embodiment 16: A compound according to any one of embodiments 2 to 13, wherein
   R² is selected from —Cl, —F, —CF₃, and —OCF₃.
Embodiment 17: A compound according to any one of embodiments 2 to 13, wherein
   R² is —OCF₃.
Embodiment 18: A compound according to any one of embodiments 2 to 13, wherein
   R² is —CF₃.
Embodiment 19: A compound according to any one of embodiments 2 to 13, wherein
   R² is —F.
Embodiment 20: A compound according to any one of embodiments 2 to 13, wherein
   R² is —Cl.
Embodiment 21: A compound according to any one of embodiments 2 to 20, wherein
   R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—Cl, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
Embodiment 22: A compound according to any one of embodiments 2 to 21, wherein
   R⁴ is -methyl, -ethyl, -isopropyl, -isobutyl, —CH₂CH₂—OCH₃, —CH₂CH₂—F, or —CH₂CH₂—NH₂.
Embodiment 23: A compound according to any one of embodiments 2 to 22, wherein
   R⁴ is -methyl, -ethyl, -isopropyl, or -isobutyl.
Embodiment 24: A compound according to any one of embodiments 2 to 23, wherein
   R⁴ is -methyl.
Embodiment 25: A compound according to any one of embodiments 2 to 23, wherein
   R⁴ is -ethyl.
Embodiment 26: A compound according to any one of embodiments 2 to 21, wherein
   R⁴ is —(CH₂)₂—OCH₃, —(CH₂)₂—F, —(CH₂)₂—Cl, —(CH₂)₂—OCF₃, —(CH₂)₂—NH₂, —(CH₂)₂—CN, —(CH₂)₂—OH, —(CH₂)₂—CF₃, —(CH₂)₂—CO₂H, —(CH₂)₂—SH, —(CH₂)₂—SCH₃, or —(CH₂)₂—S(O)₂CH₃.
Embodiment 27: A compound according to any one of embodiments 2 to 26, wherein
   R¹ is selected from hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, and —NH—C₁₋₆ alkyl.
Embodiment 28: A compound according to any one of embodiments 2 to 26, wherein
   R¹ is selected from —OCH₃, —F, —CF₃, —OCF₃, —N(CH₃)₂, —N(CH₂CH₃)₂, and —N(CH₃)(CH₂CH₃).
Embodiment 29: A compound according to any one of embodiments 2 to 26, wherein
   R¹ is selected from hydrogen, —OCH₃, and —F.
Embodiment 30: A compound according to any one of embodiments 2 to 26, wherein
   R¹ is hydrogen.
Embodiment 31: A compound according to any one of embodiments 2 to 30, wherein
   G is hydrogen, —C₁₋₈ alkyl, —C₃₋₁₀ cycloalkyl, —C₁₋₆ alkylene-C₃₋₈ cycloaklyl, heterocyclyl, or NR^{h} R^{k}, where the alkyl, alkylene, cycloalkyl, and heterocyclyl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

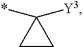

where Y³ is -cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{a};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h} R^{k},
   p) —C(O)-heterocyclyl,
   q) —NR^{h} C(O)R^{k},
   r) —OC(O)NR^{h} R^{k},
   s) —NR^{h} C(O)OR^{k},
   t) —NR^{h} C(O)NR^{h} R^{k}, or
   u) —O—(C₁₋₄ alkylene)-O—(C₁₋₄ alkylene)-N(R^{h})C(O)—OR^{k},
   where the alkylene, alkyl, cycloalkyl, and heterocyclyl groups are optionally substituted one or more times with substituents independently selected from R^{x};
R^{h} and R^{k} are independently hydrogen, C₁₋₆ alkyl, or C₃₋₁₀ cycloalkyl, where the alkyl, and cycloalkyl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
Embodiment 32: A compound according to any one of embodiments 2 to 31, wherein
   L-G is not -cyano.
Embodiment 33: A compound according to any one of embodiments 2 to 32, wherein
   L-G is —C(O)NR^{h} R^{k}.
Embodiment 34: A compound according to any one of embodiments 2 to 32, wherein
   L is —C(O)N(R⁶)— or —C(O)—O—.
Embodiment 35: A compound according to any one of embodiments 2 to 32, wherein
   L is —C(O)N(R⁶)—.
Embodiment 36: A compound according to any one of embodiments 2 to 32, wherein
   L is not —CH₂—C(O)N(R⁶)—.
Embodiment 37: A compound according to any one of embodiments 2 to 32, wherein
   L is —C(O)—O—.
Embodiment 38: A compound according to any one of embodiments 2 to 32, wherein
   L is —C(O)—.
Embodiment 39: A compound according to any one of embodiments 2 to 32, wherein
   L is —S(O)₂—.
Embodiment 40: A compound according to any one of embodiments 2 to 30, wherein
   L is heteroarylene optionally substituted one or more times with substituents independently selected from R^{x}.
Embodiment 41: A compound according to any one of embodiments 2 to 40, wherein
   R⁶ is hydrogen.
Embodiment 42: A compound according to any one of embodiments 2 to 40, wherein
   R⁶ is hydrogen or -methyl.
Embodiment 43: A compound according to any one of embodiments 2 to 42, wherein
   G is hydrogen, —C₁₋₈ alkyl, —C₃₋₁₀ cycloalkyl, or —C₁₋₆ alkylene-C₃₋₈ cycloaklyl, where the alkyl, cycloalkyl, and alkylene groups are optionally substituted one or more times with substituents independently selected from R^{x}.
Embodiment 44: A compound according to any one of embodiments 2 to 42, wherein
   G is —H, -methyl, -ethyl, -n-propyl, -isopropyl, -isobutyl, —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, or —C(Y³)(CH₃)₂, where Y³ is -cyclopropyl, —CF₃, —OCF₃, —OCH₃, —OCH₂CH₃, —F, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, —NH—C(O)—CH₃, —NH—C(O)—CH₂CH₃, or C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, or —N(CH₂CH₃)₂.
Embodiment 45: A compound according to any one of embodiments 2 to 42, wherein
   G is -methyl, -ethyl, -n-propyl, -isopropyl, or -isobutyl, where each is optionally substituted one or more times with substituents independently selected from —CF₃, —OCF₃, —OCH₃, —OCH₂CH₃, —F, —OH, —O(CH₂)₂—OH, —O(CH₂)₂—F, —SCH₃, —SCH₂CH₃, —NH—CH₃, —NH—CH₂CH₃, and —N(CH₃)₂.
Embodiment 46: A compound according to any one of embodiments 2 to 42, wherein
   G is H.
Embodiment 47: A compound according to any one of embodiments 2 to 42, wherein
   G is C₁₋₈ alkyl optionally substituted one or more times with halogen.
Embodiment 48: A compound according to any one of embodiments 2 to 42, wherein
   G is C₃₋₁₀ cycloalkyl optionally substituted one or more times with halogen.
Embodiment 49: A compound according to any one of embodiments 2 to 42, wherein
   G is heterocyclyl optionally substituted one or more times with halogen.
Embodiment 50: A compound according to any one of embodiments 2 to 42, wherein
   G is —C₁₋₆ alkylene-C₃₋₁₀ cycloalkyl optionally substituted one or more times with halogen.
Embodiment 51: A compound according to any one of embodiments 2 to 42, wherein
   G is NR^{h} R^{k}.
Embodiment 52: A compound according to any one of embodiments 2 to 42, wherein
   G is —CH₂—R^{c}.
Embodiment 53: A compound according to any one of embodiments 2 to 42, wherein
   G is —CH₂CH₂—R^{c}.
Embodiment 54: A compound according to any one of embodiments 2 to 42, wherein
   G is —CH₂CH₂CH₂—R^{c}.
Embodiment 55: A compound according to any one of embodiments 2 to 42, wherein
   G is —CH(CH₃)CH₂R^{c}.
Embodiment 56: A compound according to any one of embodiments 2 to 42, wherein
   G is —CH₂CH(R^{c})CH₃.
Embodiment 57: A compound according to any one of embodiments 2 to 42, wherein
   G is —CH(R^{c})CH₃.
Embodiment 58: A compound according to any one of embodiments 2 to 42, wherein
   G is —CH₂C(R^{c})(CH₃)₂.
Embodiment 59: A compound according to any one of embodiments 2 to 42, wherein
   G is —C(R^{c})(CH₃)₂.
Embodiment 60: A compound according to any one of embodiments 2 to 42, wherein
   G is imidazol-2-yl, thiazol-2yl, oxazol-2-yl, pyrazol1-yl, furan-2yl, thiophen-2-yl, pyrrol-1-yl, 1H-1,2,4-triazolyl-3-yl, 5-methyl-1H-1,2,4-triazolyl-3-yl, —(CH₂)₁₋₃-(imidazol-2-yl), —(CH₂)₁₋₃-(thiazol-2yl), —(CH₂)₁₋₃-(oxazol-2-yl), —(CH₂)₁₋₃-(pyrazol1-yl), —(CH₂)₁₋₃-(furan-2yl), —(CH₂)₁₋₃-(thiophen-2-yl), —(CH₂)₁₋₃-(pyrrol-1-yl), —(CH₂)₁₋₃-(1H-1,2,4-triazolyl-3-yl), or —(CH₂)₁₋₃-(5-methyl-1H-1,2,4-triazolyl-3-yl).
Embodiment 61: A compound according to any one of embodiments 2 to 60, wherein
   the compound is in its free (non-salted) form.
Embodiment 62: A compound according to any one of embodiments 2 to 60, wherein
   the compound is in the form of a pharmaceutically acceptable salt.
Embodiment 63: A compound according to any one of embodiments 1 to 62, wherein
   any “heterocyclyl” group present in the compound is selected from the group consisting of: azetidin-1-yl, azetidin-2-yl, azetidin-3-yl, pyrrolidin-1-yl, pyrrolidin-2-yl, pyrrolidin-3-yl, tetrahydrofuran-2-yl, tetrahydrofuran-3-yl, tetrahydrothiophen-2-yl, tetrahydrothiophen-3-yl, pyrazolidin-1-yl, pyrazolidin-3-yl, pyrazolidin-4-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolodin-5-yl, isothiazolidin-2-yl, isothiazolidin-3-yl, isothiazolidin-4-yl, isothiazolodin-5-yl, 1,3-dioxolan-2-yl, 1,3-dioxolan-4-yl, 1,3-oxathiolan-2-yl, 1,3-oxathiolane-4-yl, 1,3-oxathiolan-5-yl, 1,2-dithiolan-3-yl, 1,2-dithiolan-4-yl, 1,3-dithiolan-2-yl, 1,3-dithiolan-4-yl, piperidin-1-yl, piperidin-2-yl, piperidin-3-yl, piperidin-4-yl, tetrahydropyran-2-yl, tetrahydropyran-3-yl, tetrahydropyran-4-yl, thian-2-yl, thian-3-yl, thian-4-yl, piperazin-1-yl, piperazin-2-yl, morpholin-2-yl, morpholin-3-yl, morpholin-4-yl, thiomorpholin-2-yl, thiomorpholin-3-yl, thiomorpholin-4-yl, 1,4-dioxan-2-yl, 1,3-dioxan-2-yl, 1,3-dioxan-4-yl, 1,3-dioxan-5-yl, 1,4-dithian-2-yl, 1,3-dithian-2-yl, 1,3-dithian-4-yl, 1,3-dithian-5-yl, 1,2-dithian-3-yl, 1,2-dithian-4-yl, azepan-1-yl, azepan-2-yl, azepan-3-yl, and azepan-4-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, and —C(O)N(C₁₋₄ alkyl)₂, and where any nitrogen atom in any of these named rings may optionally be oxidized when chemically feasible, and where any sulfur atom in any of these named rings may optionally be oxidized once or twice when chemically feasible.
Embodiment 64: A compound according to any one of embodiments 1 to 63, wherein
   any “heteroaryl” group present in the compound is selected from the group consisting of: 1H-pyrrol-1-yl, 1H-pyrrol-2-yl, 1H-pyrrol-3-yl, furan-2-yl, furan-3-yl, thiophen-2-yl, thiophen-3-yl, 1H-imidazol-1-yl, 1H-imidazol-2-yl, 1H-imidazol-4-yl, 1H-imidazol-5-yl, 1H-pyrazol-1-yl, 1H-pyrazol-3-yl, 1H-pyrazol-4-yl, 1H-pyrazol-5-yl, oxazol-2-yl, oxazol-4-yl, oxazol-5-yl, thiazol-2-yl, thiazol-4-yl, thiazol-5-yl, isoxazol-3-yl, isoxazol-4-yl, isoxazol-5-yl, isothiazol-3-yl, isothiazol-4-yl, isothiazol-5-yl, 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl, 1H-1,2,4-triazol-5-yl, furazan-3-yl, pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1H-indol-1-yl, 1H-indol-2-yl, 1H-indol-3-yl, 2H-isoindol-1-yl, 2H-isoindol-2-yl, quinolin-2-yl, quinolin-3-yl, quinolin-4-yl, isoquinolin-1-yl, isoquinolin-3-yl, isoquinolin-4-yl, benzoxazol-2-yl, benzothiazol-2-yl, 1H-benzimidazol-1-yl, 1H-benzimidazol-2-yl, benzofuran-2-yl, benzofuran-3-yl, benzothiophen-2-yl, and benzothiophen-3-yl, where each of these named rings may optionally be substituted one or more times with substituents independently selected from halogen, —NH₂, cyano, carboxy, C₁₋₄ alkyl, C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, —C(O)N(C₁₋₄ alkyl)₂, and phenyl.
Embodiment 65: A compound according to any one of embodiments 1 to 64, wherein
   any “heteroarylene” group present in the compound is selected from the group consisting of: 1H-pyrrol-2,5-diyl, furan-2,5-diyl, thiophen-2,5-diyl, 1H-imidazol-2,4-diyl, 1H-imidazol-2,5-diyl, oxazol-2,4-diyl, oxazol-2,5-diyl, thiazol-2,4-diyl, thiazol-2,5-diyl, 1H-1,2,4-triazol-3,5-diyl, and 2H-isoindol-1,3-diyl, where each of these named rings may optionally be substituted one or more times with substituents independently selected from halogen, —NH₂, cyano, carboxy, —C₁₋₄ alkyl, —C₃₋₁₀ cycloalkyl, hydroxyl, thiol, —CF₃, —OCF₃, —O—C₁₋₄ alkyl, —NH—C₁₋₄ alkyl, —N(C₁₋₄ alkyl)₂, —S—C₁₋₄ alkyl, —S(O)₂—C₁₋₄ alkyl, —C(O)—C₁₋₄ alkyl, —C(O)O—C₁₋₄ alkyl, —C(O)NH₂, —C(O)NH—C₁₋₄ alkyl, —C(O)N(C₁₋₄ alkyl)₂, and phenyl.
Embodiment 66: A compound according to embodiment 1.
Embodiment 67: A compound according to embodiment 66, wherein
   R³ is hydrogen.
Embodiment 68: A compound according to embodiment 66, wherein
   R³ is methyl.
Embodiment 69: A compound according to embodiment 66, wherein
   R³ is ethyl.
Embodiment 70: A compound according to embodiment 66, wherein
   R³ is isopropyl.
Embodiment 71: A compound according to any one of embodiment 66 to 70, wherein X¹ is ═N—.
Embodiment 72: A compound according to any one of embodiments 66 to 70, wherein X¹ is ═CH—.
Embodiment 73: A compound according to any one of embodiments 66 to 72, wherein
   v is 0, 1 or 2.
Embodiment 74: A compound according to any one of embodiments 66 to 72, wherein
   v is 1 or 2.
Embodiment 75: A compound according to any one of embodiments 66 to 72, wherein
   v is 1.
Embodiment 76: A compound according to any one of embodiments 66 to 72, wherein
   v is 1, and R² is attached at either the 5-position or the 6-position of the benzothiazole ring.
Embodiment 77: A compound according to any one of embodiments 66 to 72, wherein
   v is 1, and R² is attached at the 6-position of the benzothiazole ring.
Embodiment 78: A compound according to any one of embodiments 66 to 72, wherein
   v is 2, and one R² is attached at the 6-position of the benzothiazole ring.
Embodiment 79: A compound according to any one of embodiments 66 to 72, wherein
   v is 2, and R² is attached at the 5-position and the 6-position of the benzothiazole ring.
Embodiment 80: A compound according to any one of embodiments 66 to 79, wherein
   R² is -halogen, —C₁₋₆ alkyl, —CF₃, —OCF₃, —O—R^{f}, or —S(O)_{w}—R^{f}, where the alkyl group is optionally substituted one or more times with substituents independently selected from R^{z}.
Embodiment 81: A compound according to any one of embodiments 66 to 79, wherein
   R² is -halogen, -methyl, ethyl, isopropyl, —OCH₃, —OCH₂CH₃, —OCH(CH₃)₂, —CF₃, —OCF₃, —SCF₃, —S(O)₂—CH₃, —O-phenyl, —O-(2-pyridyl), —O-(3-pyridyl), or —O-(4-pyridyl).
Embodiment 82: A compound according to any one of embodiments 66 to 79, wherein
   R² is -halogen, -methyl, ethyl, isopropyl, —OCH₃, —OCH₂CH₃, —OCH(CH₃)₂, —CF₃, —OCF₃, —SCF₃, —S(O)₂—CH₃, or —O-(3-pyridyl).
Embodiment 83: A compound according to any one of embodiments 66 to 79, wherein
   R² is —Cl, —F, —CF₃, or —OCF₃.
Embodiment 84: A compound according to any one of embodiments 66 to 79, wherein
   R² is —OCF₃.
Embodiment 85: A compound according to any one of embodiments 66 to 79, wherein
   R² is —CF₃.
Embodiment 86: A compound according to any one of embodiments 66 to 79, wherein
   R² is —F.
Embodiment 87: A compound according to any one of embodiments 66 to 79, wherein
   R² is —Cl.
Embodiment 88: A compound according to any one of embodiments 66 to 79, wherein
   R² is —SO₂CH₃.
Embodiment 89: A compound according to any one of embodiments 66 to 79, wherein
   R² is methyl, ethyl, or isopropyl.
Embodiment 90: A compound according to any one of embodiments 66 to 79, wherein
   R² is methyl.
Embodiment 91: A compound according to any one of embodiments 66 to 79, wherein
   R² is —OCH₂CH₃.
Embodiment 92: A compound according to any one of embodiments 66 to 79, wherein
   R² is —O-phenyl.
Embodiment 93: A compound according to any one of embodiments 66 to 79, wherein
   R² is —O-(2-pyridyl), —O-(3-pyridyl), or —O-(4-pyridyl).
Embodiment 94: A compound according to any one of embodiments 66 to 79, wherein
   R² is —O-(3-pyridyl).
Embodiment 95: A compound according to any one of embodiments 66 to 94, wherein
   R⁴ is -methyl, -ethyl, -n-propyl, -isopropyl, -n-butyl, -sec-butyl, -isobutyl, -tert-butyl, —(CH₂)₁₋₂—OCH₃, —(CH₂)₁₋₂—F, —(CH₂)₁₋₂—Cl, —(CH₂)₁₋₂—OCF₃, —(CH₂)₁₋₂—NH₂, —(CH₂)₁₋₂—CN, —(CH₂)₁₋₂—OH, —(CH₂)₁₋₂—CF₃, —(CH₂)₁₋₂—CO₂H, —(CH₂)₁₋₂—SH, —(CH₂)₁₋₂—SCH₃, —(CH₂)₁₋₂—S(O)₂CH₃, —(CH₂)₁₋₂—OCH₂CH₃, —(CH₂)₁₋₂—SCH₂CH₃, —(CH₂)₁₋₂—S(O)₂CH₂CH₃, —(CH₂)₁₋₂—NH—CH₃, or —(CH₂)₁₋₂—N(CH₃)₂.
Embodiment 96: A compound according to any one of embodiments 66 to 94, wherein
   R⁴ is -methyl, -ethyl, -isopropyl, -isobutyl, —CH₂CH₂—OCH₃, —CH₂CH₂—F, —CH₂CH₂—NH₂, or —CH₂CH₂—NH—CH₃.
Embodiment 97: A compound according to any one of embodiments 66 to 94, wherein
   R⁴ is -methyl, -ethyl, -isopropyl, or -isobutyl.
Embodiment 98: A compound according to any one of embodiments 66 to 94, wherein
   R⁴ is methyl.
Embodiment 99: A compound according to any one of embodiments 66 to 94, wherein
   R⁴ is -ethyl.
Embodiment 100: A compound according to any one of embodiments 66 to 94, wherein
   R⁴ is -isopropyl.
Embodiment 101: A compound according to any one of embodiments 66 to 94, wherein
   R⁴ is -isobutyl.
Embodiment 102: A compound according to any one of embodiments 66 to 94, wherein
   R⁴ is —CH₂CH₂—OCH₃.
Embodiment 103: A compound according to any one of embodiments 66 to 94, wherein
   R⁴ is —CH₂CH₂—F.
Embodiment 104: A compound according to any one of embodiments 66 to 94, wherein
   R⁴ is-CH₂CH₂—NH₂.
Embodiment 105: A compound according to any one of embodiments 66 to 94, wherein
   R⁴ is —CH₂CH₂—NH—CH₃.
Embodiment 106: A compound according to any one of embodiments 66 to 105, wherein
   R¹ is hydrogen, —OCH₃, —F, —Cl, —NH₂, -cyano, —OH, —CF₃, —OCF₃, —SH, —S—C₁₋₆ alkyl, —S(O)₂—C₁₋₆ alkyl, —CO₂H, —NH—C₁₋₆ alkyl, —N(C₁₋₆ alkyl)₂, or —NH—C₁₋₆ alkyl.
Embodiment 107: A compound according to any one of embodiments 66 to 105, wherein
   R¹ is —OCH₃, —F, —CF₃, —OCF₃, —N(CH₃)₂, —N(CH₂CH₃)₂, or —N(CH₃)(CH₂CH₃).
Embodiment 108: A compound according to any one of embodiments 66 to 105, wherein
   R¹ is hydrogen, —OCH₃, or —F.
Embodiment 109: A compound according to any one of embodiments 66 to 105, wherein
   R¹ is hydrogen.
Embodiment 110: A compound according to any one of embodiments 66 to 105, wherein
   R¹ is —F.
Embodiment 111: A compound according to any one of embodiments 66 to 105, wherein
   R¹ is —OCH₃.
Embodiment 112: A compound according to any one of embodiments 66 to 105 wherein
   R¹ is —N(CH₂CH₃)₂.
Embodiment 113: A compound according to any one of embodiments 66 to 112, wherein
   G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, or NR^{h} R^{k}, where the alkyl, alkylene, cycloalkyl, and heterocyclyl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or

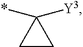

where Y³ is cyclopropyl, —CF₃, —OCF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, or heterocyclylene optionally substituted one or more times with substituents independently selected from R^{x}; or the group -L-G is -cyano;
R¹ is hydrogen or R^{a};
R^{c} is
   a) -halogen,
   b) —C₁₋₆ alkyl,
   c) —C₃₋₁₀ cycloalkyl,
   d) -heterocyclyl,
   e) -cyano,
   f) —CF₃,
   g) —OCF₃,
   h) —O—R^{h},
   i) —S(O)_{w}—R^{h},
   j) —S(O)₂O—R^{h},
   k) —NR^{h}R^{k},
   l) —C(O)—R^{h},
   m) —C(O)—O—R^{h},
   n) —OC(O)—R^{h},
   o) —C(O)NR^{h} R^{k},
   p) —C(O)-heterocyclyl,
   q) —NR^{h} C(O)R^{k},
   r) —OC(O)NR^{h} R^{k},
   s) —NR^{h}C(O)OR^{k},
   t) —NR^{h}C(O)NR^{h} R^{k},
   u) —NR^{h}S(O)_{w}R^{k}, or
   v) —O—(C₁₋₄ alkylene)-O—(C₁₋₄ alkylene)-N(R^{h})C(O)—OR^{k},
   where the alkylene, alkyl, cycloalkyl, and heterocyclyl groups are optionally substituted one or more times with substituents independently selected from R^{x};
R^{h} and R^{k} independently are hydrogen, C₁₋₆ alkyl, C₃₋₁₀ cycloalkyl, or heterocyclyl, where the alkyl, cycloalkyl, and heterocyclyl groups are optionally substituted one or more times with substituents independently selected from R^{x}; or, if R^{h} and R^{k} are both attached to the same nitrogen atom, together with that nitrogen atom may optionally form a heterocyclic ring selected from azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x}; and
R^{x} is R^{y}.
Embodiment 114: A compound according to any one of embodiments 66 to 112, wherein
   L-G is not -cyano.
Embodiment 115: A compound according to any one of embodiments 66 to 112, wherein
   L is —C(O)N(R⁶)—.
Embodiment 116: A compound according to embodiment 115 wherein R⁶ is hydrogen.
Embodiment 117: A compound according to embodiment 115 wherein R⁶ is methyl.
Embodiment 118: A compound according to embodiment 117 wherein G is —N(CH₃)₂.
Embodiment 119: A compound according to any one of embodiments 66 to 112, wherein
   L-G is —C(O)NR^{h} R^{k}.
Embodiment 120: A compound according to embodiment 119, wherein
   NR^{h} R^{k} is pyrrolidino, piperidino, piperazino, 4-methyl-piperazino, or morpholino, where each of the foregoing is optionally substituted once with —(CH₂)₁₋₃—OH.
Embodiment 121: A compound according to embodiment 120, wherein
   NR^{h} R^{k} is pyrrolidino, 4-(2-hydroxyethyl)-piperazino, or 4-(3-hydroxypropyl)-piperidino.
Embodiment 122: A compound according to embodiment 119, wherein
   NR^{h} R^{k} is N[(CH₂)₂—OH]₂.
Embodiment 123: A compound according to any one of embodiments 66 to 114, wherein
   L is not —CH₂—C(O)N(R⁶)—.
Embodiment 124: A compound according to any one of embodiments 66 to 123, wherein
   L is not heterocyclylene.
Embodiment 125: A compound according to any one of embodiments 66 to 112, wherein
   L is —S(O)₂—.
Embodiment 126: A compound according to embodiment 125, wherein
   G is methyl or —CF₃.
Embodiment 127: A compound according to any one of embodiments 66 to 112, wherein
   L is heteroarylene optionally substituted one or more times with substituents independently selected from R^{x}.
Embodiment 128: A compound according to embodiment 127, wherein
   L-G is imidazol-2-yl, 1,2,4-triazol-3-yl, or 5-methyl-1,2,4-triazol-3-yl.
Embodiment 129: A compound according to any one of embodiments 66 to 112, wherein
   L is —C(O)—O—.
Embodiment 130: A compound according to embodiment 129, wherein
   G is hydrogen, or —C₁₋₈ alkyl, where the alkyl group is optionally substituted one or more times with substituents independently selected from R^{c}.
Embodiment 131: A compound according to embodiment 130, wherein
   G is methyl or ethyl.
Embodiment 132: A compound according to embodiment 130, wherein
   G is hydrogen.
Embodiment 133: A compound according to any one of embodiments 66 to 116, wherein
   G is —C₁₋₈ alkyl, —C₃₋₁₀ cycloalkyl, —C₁₋₆ alkylene-C₃₋₁₀ cycloaklyl, heterocyclyl, or —C₁₋₆ alkylene-C₃₋₁₀ heterocyclyl, where the alkyl, alkylene, cycloalkyl, and heterocyclyl groups are optionally substituted one or more times with substituents independently selected from R^{c}.
Embodiment 134: A compound according to embodiment 133, wherein
   G is —C₁₋₈ alkyl optionally substituted one or more times with substituents independently selected from R^{c}.
Embodiment 135: A compound according to embodiment 134, wherein
   G is methyl, ethyl, isopropyl, n-propyl, n-butyl, sec-butyl, or isobutyl.
Embodiment 136: A compound according to embodiment 134, wherein
   G is methyl, ethyl, or n-propyl.
Embodiment 137: A compound according to embodiment 134, wherein
   G is 2-fluoroethyl, 2,2-difluoroethyl, or 2,2,2-trifluoroethyl.
Embodiment 138: A compound according to embodiment 134, wherein
   G is 2-cyanoethyl.
Embodiment 139: A compound according to embodiment 134, wherein
   G is —C₁₋₈ alkyl substituted once by —C(O)—O—R^{h}.
Embodiment 140: A compound according to embodiment 139, wherein
   G is —CH₂—C(O)—O—R^{h}.
Embodiment 141: A compound according to embodiment 140, wherein
   R^{h} is hydrogen or methyl.
Embodiment 142: A compound according to embodiment 139, wherein
   G is —CH₂CH₂—C(O)—O—R^{h}.
Embodiment 143: A compound according to embodiment 142, wherein
   R^{h} is hydrogen or methyl.
Embodiment 144: A compound according to embodiment 139, wherein
   G is —C(CH₃)₂—C(O)—O—R^{h}.
Embodiment 145: A compound according to embodiment 144, wherein
   R^{h} is hydrogen or methyl.
Embodiment 146: A compound according to embodiment 139, wherein
   G is —CH(CH₃)—C(O)—O—R^{h}.
Embodiment 147: A compound according to embodiment 146, wherein
   R^{h} is hydrogen or methyl.
Embodiment 148: A compound according to embodiment 134, wherein
   G is —C₁₋₈ alkyl substituted once by —C(O)NR^{h} R^{k}.
Embodiment 149: A compound according to embodiment 148, wherein
   G is CH₂—C(O)—NR^{h} R^{k}.
Embodiment 150: A compound according to embodiment 149, wherein
   NR^{h} R^{k} is methylamino, dimethylamino, or diethylamino.
Embodiment 151: A compound according to embodiment 149, wherein
   NR^{h} R^{k} is thiomorpholino or 1,1-dioxothiomorpholino.
Embodiment 152: A compound according to embodiment 149, wherein
   NR^{h} R^{k} is morpholino, pyrrolidino, piperidino, piperazino, or 4-methylpiperazino.
Embodiment 153: A compound according to embodiment 149, wherein
   NR^{h} R^{k} is pyrrolidino, 3-hydroxy-pyrrolidino, 3-methoxy-pyrrolidino, 3-amino-pyrrolidino, 3-(methylamino)-pyrrolidino, 3-(dimethylamino)-pyrrolidino, 2-(hydroxymethyl)-pyrrolidino, 2-(dimethylaminocarbonyl)-pyrrolidino or 3,4-dihydroxy-pyrrolidino.
Embodiment 154: A compound according to embodiment 149, wherein
   NR^{h} R^{k} is piperazino, 4-methylpiperazino, 4-(methylsulfonyl)-piperazino, or 4-(dimethylaminosulfonyl)-piperazino.
Embodiment 155: A compound according to embodiment 149, wherein
   NR^{h} R^{k} is piperidino, 3-hydroxypiperidino, 4-hydroxypiperidino, 2-(hydroxymethyl)-piperidino, 3-(hydroxymethyl)-piperidino, 4-(hydroxymethyl)-piperidino, 3-methoxy-piperidino, 4-(methoxymethyl)-piperidino, 4-(fluoromethyl)-piperidino, 4-(trifluoromethyl)-piperidino, 4-cyano-piperidino, 4-carbamoyl-piperidino, 4-(methylamino)-piperidino, 4-(dimethylamino)-piperidino, 4-(methylaminomethyl)-piperidino, or 4-(dimethylaminomethyl)-piperidino.
Embodiment 156: A compound according to embodiment 149, wherein
   NR^{h} R^{k} is NHR^{k}, where R^{k} is 2-hydroxypropyl, 2-(methylsulfonyl)-ethyl, tetrahydrofuran-3-yl, tetrahydropyran-4-yl, 1-methylpiperidin-4-yl, piperidin-3-yl, or 1-methylpiperidin-3-yl.
Embodiment 157: A compound according to embodiment 149, wherein
   NR^{h} R^{k} is N(CH₃)R^{k}, where R^{k} is 2-hydroxyethyl, tetrahydropyran-4-yl, pyrrolidin-3-yl, 1-methylpyrrolidin-3-yl, or piperazin-3-yl.
Embodiment 158: A compound according to embodiment 149, wherein
   NR^{h} R^{k} is N(CH₂CH₂OH)₂.
Embodiment 159: A compound according to embodiment 148, wherein
   G is —(CH₂)₂₋₃—C(O)—N(CH₃)₂.
Embodiment 160: A compound according to embodiment 148, wherein
   G is —(CH₂)₃—C(O)-(4-methylpiperazino).
Embodiment 161: A compound according to embodiment 148, wherein
   G is —CH(CH₃)—C(O)—NR^{h} R^{k}, where NR^{h} R^{k} is methylamino, dimethylamino, 4-methylpiperazino, or morpholino.
Embodiment 162: A compound according to embodiment 148, wherein
   G is —C(CH₃)₂—C(O)—N(CH₃)₂.
Embodiment 163: A compound according to embodiment 134, wherein
   G is —CH—[C(O)—N(CH₃)₂]—[CH₂OH], —CH—[C(O)—N(CH₃)₂]—[(CH₂)₄₋NH₂], or —CH—[C(O)—N(CH₃)₂]—[(CH₂)₄—N(CH₃)₂].
Embodiment 164: A compound according to embodiment 134, wherein
   G is —C₁₋₈ alkyl substituted once by —O—R^{h}.
Embodiment 165: A compound according to embodiment 164, wherein
   G is —(CH₂)₂—O—R^{h}.
Embodiment 166: A compound according to embodiment 165, wherein
   R^{h} is hydrogen, methyl, or ethyl.
Embodiment 167: A compound according to embodiment 165, wherein
   R^{h} is trifluoromethyl, 2-fluoroethyl, 3-fluoropropyl, or 2,2-difluoroethyl.
Embodiment 168: A compound according to embodiment 165, wherein
   R^{h} is tetrahydrofuran-2-ylmethyl.
Embodiment 169: A compound according to embodiment 165, wherein
   R^{h} is 2-hydroxy ethyl.
Embodiment 170: A compound according to embodiment 165, wherein
   R^{h} is 3-hydroxypropyl.
Embodiment 171: A compound according to embodiment 165, wherein
   R^{h} is 2-methoxyethyl.
Embodiment 172: A compound according to embodiment 165, wherein
   R^{h} is 2-(2-hydroxyethoxy)-ethyl.
Embodiment 173: A compound according to embodiment 165, wherein
   R^{h} is 2-hydroxypropyl or 1-hydroxyprop-2-yl.
Embodiment 174: A compound according to embodiment 165, wherein
   R^{h} is 2-cyanoethyl, 2-(methylcarbonylamino)-ethyl, or 2-(methylsulfonylamino)-ethyl.
Embodiment 175: A compound according to embodiment 165, wherein
   R^{h} is 2-aminoethyl, 2-(methylamino)-ethyl, or 2-(dimethylamino)-ethyl.
Embodiment 176: A compound according to embodiment 165, wherein
   R^{h} is carbarnoylmethyl.
Embodiment 177: A compound according to embodiment 164, wherein
   G is —(CH₂)₃—O—R^{h}.
Embodiment 178: A compound according to embodiment 177, wherein
   R^{h} is hydrogen, methyl, or ethyl.
Embodiment 179: A compound according to embodiment 177, wherein
   R^{h} is 2-hydroxy ethyl.
Embodiment 180: A compound according to embodiment 164, wherein
   G is —(CH₂)₄—OH, —(CH₂)₅—OH, —CH₂C(CH₃)₂—OH, —CH₂C(CH₃)₂—OCH₃, —CH₂C(CH₃)₂—CH₂—OH, —CH(CH₃)—CH₂—OCH₃, —(CH₂)₃C(CH₃)₂—CH₂—OH, —(CH₂)₂CH(CH₃)—CH₂—OH, or —(CH₂)₂CH(CH₃)—OH.
Embodiment 181: A compound according to embodiment 164, wherein
   G is —CH₂CH(CH₃)—O—R^{h}
Embodiment 182: A compound according to embodiment 181, wherein
   R^{h} is hydrogen, methyl, or ethyl.
Embodiment 183: A compound according to embodiment 134, wherein
   G is —CH₂—CH(OH)—CH₂—OH.
Embodiment 184: A compound according to embodiment 134, wherein
   G is —C₁₋₈ alkyl substituted once by —NR^{h}R^{k}.
Embodiment 185: A compound according to embodiment 184, wherein
   G is —(CH₂)₂—NR^{h}R^{k}
Embodiment 186: A compound according to embodiment 185, wherein
   NR^{h}R^{k} is amino, methylamino, or dimethylamino.
Embodiment 187: A compound according to embodiment 185, wherein
   NR^{h}R^{k} is methylcarbonylamino.
Embodiment 188: A compound according to embodiment 185, wherein
   NR^{h}R^{k} is (dimethylamino)methylcarbonylamino, hydroxymethylcarbonylamino, or 1-hydroxyethylcarbonylamino.
Embodiment 189: A compound according to embodiment 185, wherein
   NR^{h}R^{k} is methylsulfonylamino.
Embodiment 190: A compound according to embodiment 185, wherein
   NR^{h}R^{k} is piperidino, 4-hydroxypiperidino, or 3-hydroxypiperidino.
Embodiment 191: A compound according to embodiment 185, wherein
   NR^{h}R^{k} is piperidino, 4,4-difluoropiperidino, or 3,3-difluoropiperidino.
Embodiment 192: A compound according to embodiment 185, wherein
   NR^{h}R^{k} is 2-oxo-pyrrolidino, 2-oxo-imidazolidino, or 3-oxo-piperazino.
Embodiment 193: A compound according to embodiment 185, wherein
   NR^{h}R^{k} is piperazino, 4-methylpiperazino, morpholino, or 1,1-dioxo-thiomorpholino.
Embodiment 194: A compound according to embodiment 184, wherein
   G is —(CH₂)₃—NR^{h}R^{k}
Embodiment 195: A compound according to embodiment 194, wherein
   NR^{h}R^{k} is amino, dimethylamino, or diethylamino.
Embodiment 196: A compound according to embodiment 194, wherein
   NR^{h}R^{k} is piperidino, 4-methylpiperazino, or morpholino.
Embodiment 197: A compound according to embodiment 184, wherein
   G is —(CH₂)₄—NR^{h}R^{k}
Embodiment 198: A compound according to embodiment 197, wherein
   NR^{h}R^{k} is amino, dimethylamino, or diethylamino.
Embodiment 199: A compound according to embodiment 133, wherein
   G is —C₁₋₆ alkylene-heterocyclyl, where the alkylene and heterocyclyl groups are optionally substituted one or more times with substituents independently selected from R^{c}.
Embodiment 200: A compound according to embodiment 199, wherein
   G is —CH₂-heterocyclyl, where the heterocyclyl group is optionally substituted once with a substituent selected from R^{c}.
Embodiment 201: A compound according to embodiment 200, wherein
   the heterocyclyl group is tetrahydropyran-4-yl, tetrahydrofuran-2-yl, 1,4-dioxan-2-yl, morpholin-2-yl, tetrahydropyran-2-yl, piperidin-4-yl, 1-(2-hydroxyethyl)-piperidin-4-yl, 1-(dimethylaminomethylcarbonyl)-piperidin-4-yl, piperazin-2-yl, or 1-methyl-piperazin-2-yl.
Embodiment 202: A compound according to embodiment 133, wherein
   G is C₃₋₁₀ cycloalkyl optionally substituted one or more times with substituents independently selected from R^{c}.
Embodiment 203: A compound according to embodiment 202, wherein
   G is 4-hydroxy-cyclohexyl, 4-carboxy-cyclohexyl, or 4-(dimethylaminocarbonyl)-cyclohexyl.
Embodiment 204: A compound according to embodiment 202, wherein
   G is 1-carboxy-cyclopropyl, 1-(ethoxycarbonyl)-cyclopropyl, or 1-(dimethylamino-carbonyl)-cyclopropyl.
Embodiment 205: A compound according to embodiment 133, wherein
   G is C₁₋₆ alkylene-C₃₋₁₀ cycloalkyl, where the alkylene and cycloalkyl groups are optionally substituted one or more times with substituents independently selected from R^{c}.
Embodiment 206: A compound according to embodiment 205, wherein
   G is —CH₂-(4-hydroxy-cyclohexyl).
Embodiment 207: A compound according to embodiment 205, wherein
   G is —(CH₂)₂-(4-hydroxy-cyclohexyl).
Embodiment 208: A compound according to embodiment 205, wherein
   G is —CH₂-[4-(hydroxymethyl)-cyclohexyl].
Embodiment 209: A compound according to embodiment 133, wherein
   G is heterocyclyl optionally substituted one or more times with substituents independently selected from R^{c}.
Embodiment 210: A compound according to embodiment 209, wherein
   G is piperidin-4-yl, 1-methyl-piperidin-4-yl, 1-carboxy-piperidin-4-yl, 1-(methylsulfonyl)-piperidin-4-yl, 1-(2-hydroxyethyl)-piperidin-4-yl, 1-(dimethyl-aminocarbonyl)piperidin-4-yl, or 1-(dimethylaminomethylcarbonyl)-piperidin-4-yl.
Embodiment 211: A compound according to embodiment 209, wherein
   G is piperidin-3-yl or 1-(dimethylaminomethylcarbonyl)-piperidin-3-yl.
Embodiment 212: A compound according to embodiment 209, wherein
   G is 1,1-dioxo-tetrahydrothiophen-3-yl.
Embodiment 213: A compound according to embodiment 209, wherein
   G is pyrrolidin-3-yl, 1-methyl-pyrrolidin-3-yl, 1-(2-hydroxyethyl)-pyrrolidin-3-yl, 1-(2-hydroxypropyl)-pyrrolidin-3-yl, 1-(2-hydroxy-2-methylpropyl)-pyrrolidin-3-yl, 1-(1-hydroxyethylcarbonyl)-pyrrolidin-3-yl, 1-(2-carboxyethyl)-pyrrolidin-3-yl, or 1-(2-methylsulfonylamino-ethyl)-pyrrolidin-3-yl.
Embodiment 214: A compound according to embodiment 134, wherein
   G is —C₁₋₈ alkyl substituted once by —S—R^{h}.
Embodiment 215: A compound according to embodiment 214, wherein
   G is —(CH₂)₂—S—R^{h}.
Embodiment 216: A compound according to embodiment 215, wherein
   R^{h} is methyl or ethyl.
Embodiment 217: A compound according to embodiment 215, wherein
   R^{h} is 2-hydroxy ethyl.
Embodiment 218: A compound according to embodiment 214, wherein
   G is —(CH₂)₃—S—R^{h}.
Embodiment 219: A compound according to embodiment 218, wherein
   R^{h} is methyl.
Embodiment 220: A compound according to embodiment 134, wherein
   G is —C₁₋₈ alkyl substituted once by —SO₂—R^{h}.
Embodiment 221: A compound according to embodiment 220, wherein
   G is —(CH₂)₂—SO₂—R^{h}
Embodiment 222: A compound according to embodiment 221, wherein
   R^{h} is methyl or ethyl.
Embodiment 223: A compound according to embodiment 221, wherein
   R^{h} is 2-hydroxy ethyl.
Embodiment 224: A compound according to embodiment 220, wherein
   G is —(CH₂)₃—SO₂—R^{h}
Embodiment 225: A compound according to embodiment 224, wherein
   R^{h} is methyl.
Embodiment 226: A compound according to embodiment 133 wherein
   G is —CH(CH₃)—NR^{h}R^{k}, where NR^{h}R^{k} is pyrrolidino, piperidino, 4-methyl-piperazino, morpholino, or dimethylamino.
Embodiment 227: A compound according to embodiment 133 wherein
   G is 1-(2-hydroxypropyl)-pyrrolodin-3-yl or 1-(1-hydroxyethylcarbonyl)-pyrrolidin-3-yl.
Embodiment 228: A compound according to embodiment 133 wherein
   G is 1-(dimethylaminomethylcarbonyl)-piperidin-4-yl.
Embodiment 229: A compound according to embodiment 133 wherein
   G is —(CH₂)₃₋₅—OH.
Embodiment 230: A compound according to embodiment 133 wherein
   G is 4-hydroxy-cyclohexylmethyl.
Embodiment 231: A compound according to embodiment 133 wherein
   G is —(CH₂)₂—NHC(O)—CH₂—N(CH₃)₂.
Embodiment 232: A compound according to embodiment 133 wherein
   G is 4-hydroxy-cyclohexylmethyl.
Embodiment 233: A compound according to embodiment 133 wherein
   G is —CH₂—C(O)—NR^{h}R^{k}, where NR^{h}R^{k} is 3-hydroxy-pyrrolidino or 3-(dimethyl-amino)-pyrrolidino.
Embodiment 234: A compound according to embodiment 133 wherein
   G is —CH₂—C(O)—NR^{h}R^{k}, where NR^{h}R^{k} is morpholino.
Embodiment 235: A compound according to embodiment 133 wherein
   G is —CH₂—C(O)—NR^{h}R^{k}, where NR^{h}R^{k} is 4-hydroxy-piperidino, 4-methoxy-piperidino, 4-(hydroxymethyl)-piperidino, 3-hydroxy-piperidino, 3-methoxy-piperidino, 3-(hydroxymethyl)-piperidino, or 4,4-difluoropiperidino.
Embodiment 236: A compound according to embodiment 133 wherein
   G is —CH₂—C(O)—NR^{h}R^{k} where NR^{h}R^{k} is dimethylamino.
Embodiment 237: A compound according to embodiment 133 wherein
   G is —(CH₂)₂—O—(CH₂)₂—OH.
Embodiment 238: A compound according to embodiment 133 wherein
   G is —(CH₂)₂—O—(CH₂)₂—OCH₃.
Embodiment 239: A compound according to embodiment 133 wherein
   G is —CH₂—CH(CH₃)—OH.
Embodiment 240: A compound according to any one of embodiments 66 to 112, wherein
   L is C(O)NH, and G is C₁₋₈ alkyl substituted once by a heteroaryl group, where the heteroaryl group is optionally substituted one or more times with substituents independently selected from R^{x}.
Embodiment 241: A compound according to embodiment 240, wherein
   G is —CH₂-(2-furyl), —CH₂-(2-thienyl), —CH₂-(2-oxazolyl), or —CH₂-(2-thiazolyl).
Embodiment 242: A compound according to embodiment 240, wherein
   G is —(CH₂)₂₋₃-(1-pyrrolyl), —(CH₂)₂₋₃-(1-pyrazolyl), or —(CH₂)₂₋₃-(1-imidazolyl).
Embodiment 243: A compound according to any one of embodiments 66 to 112, wherein
   L is C(O)NH, and G is C₁₋₈ alkyl substituted once by a phenyl group, where the phenyl group is optionally substituted one or more times with substituents independently selected from R^{x}.
Embodiment 244: A compound according to embodiment 243, wherein
   G is —(—CH₂)₁₋₂-(4-hydroxyphenyl) or —(—CH₂)₁₋₂-(4-methoxy-3-hydroxyphenyl).
Embodiment 245: A compound according to any one of embodiments 66 to 112, wherein
   L is C(O)NH, and G is —CH₂—C(O)NH—CH₂-(4-hydroxyphenyl).
Embodiment 246: A compound according to any one of embodiments 66 to 112, wherein
   L is C(O)NH, and G is —CH₂—C(O)-[4-(pyrimidin-2-yloxy)-piperidino].
Embodiment 247: A compound according to any one of embodiments 1 to 246, wherein X² is ═C(R¹)— and X³ is ═C(-L-G)-.
Embodiment 248: A compound according to any one of embodiments 1 to 247, wherein the compound is in the form of a free acid or a free base.
Embodiment 249: A compound according to any one of embodiments 1 to 247, wherein the compound is in the form of a pharmaceutically acceptable salt.
Embodiment 250: A compound according to embodiment 1, wherein the compound is a compound from Table A or a pharmaceutically acceptable salt thereof.

TABLE A

| No. | Name |
|---|---|
| 1 | 1-Methyl-2-(6-trifluoromethoxy-benzothiazol-2-ylamino)-1H- |
| | benzoimidazole-5-carboxylic acid methyl amide |
| 2 | 1-Methyl-2-(6-trifluoromethoxy-benzothiazol-2-ylamino)-1H- |
| | benzoimidazole-5-carboxylic acid methyl ester |
| 3 | 1-Methyl-2-(6-trifluoromethoxy-benzothiazol-2-ylamino)-1H- |
| | benzoimidazole-5-carboxylic acid |
| 4 | 1-Methyl-2-(6-trifluoromethoxy-benzothiazol-2-ylamino)-1H- |
| | benzoimidazole-5-carboxylic acid (2-ethoxy-ethyl)-amide |
| 5 | 1-Methyl-2-(6-trifluoromethoxy-benzothiazol-2-ylamino)-1H- |
| | benzoimidazole-5-carboxylic acid cyclopropylmethyl-amide |
| 6 | 1-Methyl-2-(6-trifluoromethoxy-benzothiazol-2-ylamino)-1H- |
| | benzoimidazole-5-carboxylic acid ethylamide |
| 7 | [1-Methyl-2-(6-trifluoromethoxy-benzothiazol-2-ylamino)- |
| | 1H-benzoimidazol-5-yl]-pyrrolidin-1-yl-methanone |
| 8 | 1-Methyl-2-(6-trifluoromethoxy-benzothiazol-2-ylamino)- |
| | 1H-benzoimidazole-5-carboxylic acid (2-methoxy-ethyl)-amide |
| 9 | 1-Methyl-2-(6-trifluoromethoxy-benzothiazol-2-ylamino)- |
| | 1H-benzoimidazole-5-carboxylic acid (2-fluoro-ethyl)-amide |
| 10 | 1-Methyl-2-(6-trifluoromethoxy-benzothiazol-2-ylamino)- |
| | 1H-benzoimidazole-5-carboxylic acid (2-hydroxy-ethyl)-amide |
| 11 | 1-Methyl-2-(6-trifluoromethoxy-benzothiazol-2-ylamino)- |
| | 1H-benzoimidazole-5-carboxylic acid (3-pyrazol-1-yl-propyl)- |
| | amide |
| 12 | 1-Methyl-2-(6-trifluoromethoxy-benzothiazol-2-ylamino)- |
| | 1H-benzoimidazole-5-carboxylic acid propylamide |
| 13 | 1-Methyl-2-(6-trifluoromethoxy-benzothiazol-2-ylamino)- |
| | 1H-benzoimidazole-5-carboxylic acid (3-hydroxy-propyl)- |
| | amide |
| 14 | 1-Methyl-2-(6-trifluoromethoxy-benzothiazol-2-ylamino)- |
| | 1H-benzoimidazole-5-carboxylic acid (3-ethoxy-propyl)- |
| | amide |
| 15 | 1-Methyl-2-(6-trifluoromethoxy-benzothiazol-2-ylamino)- |
| | 1H-benzoimidazole-5-carboxylic acid morpholin-4-ylamide |
| 16 | 1-Methyl-2-(6-trifluoromethoxy-benzothiazol-2-ylamino)- |
| | 1H-benzoimidazole-5-carboxylic acid (2,2,2-trifluoro-ethyl)- |
| | amide |
| 17 | 1-Methyl-2-(6-trifluoromethoxy-benzothiazol-2-ylamino)- |
| | 1H-benzoimidazole-5-carboxylic acid (tetrahydro-pyran-4- |
| | ylmethyl)-amide |
| 18 | 1-Methyl-2-(6-trifluoromethoxy-benzothiazol-2-ylamino)- |
| | 1H-benzoimidazole-5-carboxylic acid (tetrahydro-furan-2- |
| | ylmethyl)-amide |
| 19 | 1-Methyl-2-(6-trifluoromethoxy-benzothiazol-2-ylamino)-1H- |
| | benzoimidazole-5-carboxylic acid (3-methoxy-propyl)-amide |
| 20 | 1-Methyl-2-(6-trifluoromethoxy-benzothiazol-2-ylamino)-1H- |
| | benzoimidazole-5-carboxylic acid (2-methoxy-1-methyl-ethyl)- |
| | amide |
| 21 | 1-Methyl-2-(6-trifluoromethoxy-benzothiazol-2-ylamino)-1H- |
| | benzoimidazole-5-carboxylic acid (2-hydroxy-propyl)-amide |
| 22 | 1-Methyl-2-(6-trifluoromethoxy-benzothiazol-2-ylamino)-1H- |
| | benzoimidazole-5-carboxylic acid (2-methoxy-2-methyl- |
| | propyl)-amide |
| 23 | 1-Methyl-2-(6-trifluoromethyl-benzothiazol-2-ylamino)-1H- |
| | benzoimidazole-5-carboxylic acid methyl ester |
| 24 | 1-Methyl-2-(6-trifluoromethyl-benzothiazol-2-ylamino)-1H- |
| | benzoimidazole-5-carboxylic acid |
| 25 | 1-Methyl-2-(6-trifluoromethyl-benzothiazol-2-ylamino)-1H- |
| | benzoimidazole-5-carboxylic acid (2-methoxy-ethyl)-amide |
| 26 | 1-Methyl-2-(6-trifluoromethyl-benzothiazol-2-ylamino)-1H- |
| | benzoimidazole-5-carboxylic acid ethylamide |
| 27 | 1-Methyl-2-(6-trifluoromethyl-benzothiazol-2-ylamino)-1H- |
| | benzoimidazole-5-carboxylic acid (2-hydroxy-ethyl)-amide |
| 28 | 2-(6-Chloro-benzothiazol-2-ylamino)-1-methyl-1H- |
| | benzoimidazole-5-carboxylic acid methyl ester |
| 29 | 2-(6-Chloro-benzothiazol-2-ylamino)-1-methyl-1H- |
| | benzoimidazole-5-carboxylic acid |
| 30 | 2-(6-Chloro-benzothiazol-2-ylamino)-1-methyl-1H- |
| | benzoimidazole-5-carboxylic acid (2-hydroxy-ethyl)-amide |
| 31 | 2-(6-Chloro-benzothiazol-2-ylamino)-1-methyl-1H- |
| | benzoimidazole-5-carboxylic acid (2-methoxy-ethyl)-amide |
| 32 | 2-(6-Chloro-benzothiazol-2-ylamino)-1-methyl-1H- |
| | benzoimidazole-5-carboxylic acid ethylamide |
| 33 | 2-(5,6-Difluoro-benzothiazol-2-ylamino)-1-methyl-1H- |
| | benzoimidazole-5-carboxylic acid methyl ester |
| 34 | 2-(5,6-Difluoro-benzothiazol-2-ylamino)-1-methyl-1H- |
| | benzoimidazole-5-carboxylic acid |
| 35 | 2-(5,6-Difluoro-benzothiazol-2-ylamino)-1-methyl-1H- |
| | benzoimidazole-5-carboxylic acid ethylamide |
| 36 | 2-(5,6-Difluoro-benzothiazol-2-ylamino)-1-methyl-1H- |
| | benzoimidazole-5-carboxylic acid (2-hydroxy-ethyl)-amide |
| 37 | 2-(5,6-Difluoro-benzothiazol-2-ylamino)-1-methyl-1H- |
| | benzoimidazole-5-carboxylic acid (2-methoxy-ethyl)-amide |
| 38 | 3-Methyl-2-(6-trifluoromethoxy-benzothiazol-2-ylamino)- |
| | 3H-benzoimidazole-5-carboxylic acid methylamide |
| 39 | 6-Fluoro-1-methyl-2-(6-trifluoromethoxy-benzothiazol- |
| | 2-ylamino)-1H-benzoimidazole-5-carboxylic |
| | acid (2-methoxy-ethyl)-amide |
| 40 | 6-Fluoro-1-methyl-2-(6-trifluoromethoxy-benzothiazol- |
| | 2-ylamino)-1H-benzoimidazole-5-carboxylic |
| | acid methyl ester |
| 41 | 6-Fluoro-1-methyl-2-(6-trifluoromethoxy-benzothiazol- |
| | 2-ylamino)-1H-benzoimidazole-5-carboxylic acid |
| 42 | 6-Fluoro-1-methyl-2-(6-trifluoromethoxy-benzothiazol- |
| | 2-ylamino)-1H-benzoimidazole-5-carboxylic |
| | acid ethylamide |
| 43 | 1-Methyl-2-(6-trifluoromethoxy-benzothiazol-2-ylamino)- |
| | 1H-benzoimidazole-5-carboxylic acid |
| | (2-morpholin-4-yl-ethyl)-amide |
| 44 | 1-Methyl-2-(6-trifluoromethoxy-benzothiazol-2-ylamino)- |
| | 1H-benzoimidazole-5-carboxylic acid |
| | (2-trifluoromethoxy-ethyl)-amide |
| 45 | 1-Methyl-2-(6-trifluoromethoxy-benzothiazol-2-ylamino)- |
| | 1H-benzoimidazole-5-carboxylic acid |
| | (2-hydroxy-2-methyl-propyl)-amide |
| 46 | 1-Methyl-2-(6-trifluoromethoxy-benzothiazol-2-ylamino)-1H- |
| | benzoimidazole-5-carboxylic acid [2-(2-hydroxy- |
| | ethoxy)-ethyl]-amide |
| 47 | 1-Methyl-2-(6-trifluoromethoxy-benzothiazol-2-ylamino)-1H- |
| | benzoimidazole-5-carboxylic acid [2-(2-fluoro-ethoxy)- |
| | ethyl]-amide |
| 48 | 1-Methyl-2-(6-trifluoromethoxy-benzothiazol-2-ylamino)-1H- |
| | benzoimidazole-5-carboxylic acid (furan-2-ylmethyl)- |
| | amide |
| 49 | 1-Methyl-2-(6-trifluoromethoxy-benzothiazol-2-ylamino)-1H- |
| | benzoimidazole-5-carboxylic acid |
| | ([1,4]dioxan-2-ylmethyl)-amide |
| 50 | 1-Methyl-2-(6-trifluoromethoxy-benzothiazol-2-ylamino)-1H- |
| | benzoimidazole-5-carboxylic acid |
| | ((S)-2-hydroxy-propyl)-amide |
| 51 | 1-Methyl-2-(6-trifluoromethoxy-benzothiazol-2-ylamino)-1H- |
| | benzoimidazole-5-carboxylic acid |
| | ((R)-2-hydroxy-propyl)-amide |
| 52 | 1-Methyl-2-(6-trifluoromethoxy-benzothiazol-2-ylamino)-1H- |
| | benzoimidazole-5-carboxylic acid (trans-4-hydroxy- |
| | cyclohexyl)-amide |
| 53 | 1-Methyl-2-(6-trifluoromethoxy-benzothiazol-2-ylamino)-1H- |
| | benzoimidazole-5-carboxylic acid [2-(tetrahydro- |
| | furan-2-ylmethoxy)-ethyl]-amide |
| 54 | 1-Methyl-2-(6-trifluoromethoxy-benzothiazol-2-ylamino)-1H- |
| | benzoimidazole-5-carboxylic acid |
| | (2-ethoxy-propyl)-amide |
| 55 | 2-({[1-Methyl-2-(6-trifluoromethoxy-benzothiazol- |
| | 2-ylamino)-1H-benzoimidazole-5-carbonyl]-amino}- |
| | methyl)-morpholine-4-carboxylic acid tert-butyl ester |
| 56 | 1-Methyl-2-(6-trifluoromethoxy-benzothiazol-2-ylamino)-1H- |
| | benzoimidazole-5-carboxylic acid (morpholin-2- |
| | ylmethyl)-amide hydrochloride |
| 57 | 6-Fluoro-1-methyl-2-(6-trifluoromethoxy-benzothiazol- |
| | 2-ylamino)-1H-benzoimidazole-5-carboxylic |
| | acid (2-ethoxy-ethyl)-amide |
| 58 | 6-Fluoro-1-methyl-2-(6-trifluoromethoxy-benzothiazol- |
| | 2-ylamino)-1H-benzoimidazole-5-carboxylic acid |
| | dimethylcarbamoylmethyl-amide |
| 59 | 6-Fluoro-1-methyl-2-(6-trifluoromethoxy-benzothiazol- |
| | 2-ylamino)-1H-benzoimidazole-5-carboxylic acid |
| | (2-morpholin-4-yl-ethyl)-amide |
| 60 | 6-Fluoro-1-methyl-2-(6-trifluoromethoxy-benzothiazol- |
| | 2-ylamino)-1H-benzoimidazole-5-carboxylic acid |
| | (2-hydroxy-propyl)-amide |
| 61 | 6-Methoxy-1-methyl-2-(6-trifluoromethoxy-benzothiazol- |
| | 2-ylamino)-1H-benzoimidazole-5-carboxylic acid |
| | methyl ester |
| 62 | 6-Methoxy-1-methyl-2-(6-trifluoromethoxy-benzothiazol- |
| | 2-ylamino)-1H-benzoimidazole-5-carboxylic acid |
| 63 | 6-Methoxy-1-methyl-2-(6-trifluoromethoxy-benzothiazol- |
| | 2-ylamino)-1H-benzoimidazole-5-carboxylic acid |
| | dimethylcarbamoyl-methyl-amide |
| 64 | 6-Methoxy-1-methyl-2-(6-trifluoromethoxy-benzothiazol- |
| | 2-ylamino)-1H-benzoimidazole-5-carboxylic |
| | acid ethylamide |
| 65 | 6-Methoxy-1-methyl-2-(6-trifluoromethoxy-benzothiazol- |
| | 2-ylamino)-1H-benzoimidazole-5-carboxylic |
| | acid (2-ethoxy-ethyl)-amide |
| 66 | 6-Methoxy-1-methyl-2-(6-trifluoromethoxy-benzothiazol- |
| | 2-ylamino)-1H-benzoimidazole-5-carboxylic acid |
| | (2-morpholin-4-yl-ethyl)-amide |
| 67 | 6-Methoxy-1-methyl-2-(6-trifluoromethoxy-benzothiazol- |
| | 2-ylamino)-1H-benzoimidazole-5-carboxylic acid |
| | (2-methoxy-ethyl)-amide |
| 68 | 6-Methoxy-1-methyl-2-(6-trifluoromethoxy-benzothiazol- |
| | 2-ylamino)-1H-benzoimidazole-5-carboxylic acid |
| | (2-hydroxy-propyl)-amide |
| 69 | 6-Diethylamino-1-methyl-2-(6-trifluoromethoxy- |
| | benzothiazol-2-ylamino)-1H-benzoimidazole-5-carboxylic |
| | acid methyl ester |
| 70 | 6-Diethylamino-1-methyl-2-(6-trifluoromethoxy-benzothiazol- |
| | 2-ylamino)-1H-benzoimidazole-5-carboxylic acid |
| 71 | 3-Methyl-2-(6-trifluoromethoxy-benzothiazol-2- |
| | ylamino)-3H-imidazo[4,5-b]pyridine-6-carboxylic acid |
| | ethyl ester |
| 72 | 3-Methyl-2-(6-trifluoromethoxy-benzothiazol-2- |
| | ylamino)-3H-imidazo[4,5-b]pyridine-6-carboxylic acid |
| 73 | 3-Methyl-2-(6-trifluoromethoxy-benzothiazol-2- |
| | ylamino)-3H-imidazo[4,5-b]pyridine-6-carboxylic acid |
| | (2-methoxy-ethyl)-amide |
| 74 | 3-Methyl-2-(6-trifluoromethoxy-benzothiazol-2-ylamino)- |
| | 3H-imidazo[4,5-b]pyridine-6-carboxylic acid |
| | dimethylcarbamoylmethyl-amide |
| 75 | 3-Methyl-2-(6-trifluoromethoxy-benzothiazol-2-ylamino)- |
| | 3H-imidazo[4,5-b]pyridine-6-carboxylic acid (2-ethoxy- |
| | ethyl)-amide |
| 76 | 3-Methyl-2-(6-trifluoromethoxy-benzothiazol-2-ylamino)-3H- |
| | imidazo[4,5-b]pyridine-6-carboxylic acid ethylamide |
| 77 | 3-Methyl-2-(6-trifluoromethoxy-benzothiazol-2-ylamino)-3H- |
| | imidazo[4,5-b]pyridine-6-carboxylic acid (2-morpholin- |
| | 4-yl-ethyl)-amide |
| 78 | 3-Methyl-2-(6-trifluoromethoxy-benzothiazol-2-ylamino)-3H- |
| | imidazo[4,5-b]pyridine-6-carboxylic acid (2-hydroxy- |
| | propyl)-amide |
| 79 | {[1-Methyl-2-(6-trifluoromethoxy-benzothiazol-2-ylamino)- |
| | 1H-benzoimidazole-5-carbonyl]-amino}-acetic acid |
| | methyl ester |
| 80 | 1-Methyl-2-(6-trifluoromethoxy-benzothiazol-2-ylamino)- |
| | 1H-benzoimidazole-5-carboxylic acid |
| | dimethylcarbamoylmethyl-amide |
| 81 | 1-Methyl-2-(6-trifluoromethoxy-benzothiazol-2-ylamino)- |
| | 1H-benzoimidazole-5-carboxylic acid ((S)-1- |
| | ethylcarbamoyl-ethyl)-amide |
| 82 | 1-Methyl-2-(6-trifluoromethoxy-benzothiazol-2-ylamino)- |
| | 1H-benzoimidazole-5-carboxylic acid (2- |
| | dimethylamino-ethyl)-amide |
| 83 | {[1-Methyl-2-(6-trifluoromethoxy-benzothiazol-2-ylamino)- |
| | 1H-benzoimidazole-5-carbonyl]-amino}-acetic acid |
| 84 | 1-Methyl-2-(6-trifluoromethyl-benzothiazol-2-ylamino)- |
| | 1H-benzoimidazole-5-carboxylic acid methylamide |
| 85 | 1-Methyl-2-(6-trifluoromethyl-benzothiazol-2-ylamino)- |
| | 1H-benzoimidazole-5-carboxylic acid (2-ethoxy-ethyl)-amide |
| 86 | 2-(5,6-Difluoro-benzothiazol-2-ylamino)-1-methyl-1H- |
| | benzoimidazole-5-carboxylic acid methylamide |
| 87 | 2-(5,6-Difluoro-benzothiazol-2-ylamino)-1-methyl-1H- |
| | benzoimidazole-5-carboxylic acid (2-ethoxy-ethyl)-amide |
| 88 | 2-(6-Chloro-benzothiazol-2-ylamino)-1-methyl-1H- |
| | benzoimidazole-5-carboxylic acid methylamide |
| 89 | 2-(6-Chloro-benzothiazol-2-ylamino)-1-methyl-1H- |
| | benzoimidazole-5-carboxylic acid (2-ethoxy-ethyl)-amide |
| 90 | 1-Methyl-2-(6-trifluoromethoxy-benzothiazol-2- |
| | ylamino)-1H-benzoimidazole-5-carboxylic acid |
| | (1-methanesulfonyl-piperidin-4-yl)-amide |
| 91 | {[1-Methyl-2-(6-trifluoromethoxy-benzothiazol-2-ylamino)- |
| | 1H-benzoimidazole-5-carbonyl]-amino}-acetic acid |
| | tert-butyl ester |
| 92 | 4-{[1-Methyl-2-(6-trifluoromethoxy-benzothiazol-2-ylamino)- |
| | 1H-benzoimidazole-5-carbonyl]-amino}-piperidine-1- |
| | carboxylic acid tert-butyl ester |
| 93 | 1-Methyl-2-(6-trifluoromethoxy-benzothiazol-2-ylamino)- |
| | 1H-benzoimidazole-5-carboxylic acid piperidin-4-ylamide |
| | hydrochloride |
| 94 | 3-{[1-Methyl-2-(6-trifluoromethoxy-benzothiazol-2-ylamino)- |
| | 1H-benzoimidazole-5-carbonyl]-amino}-piperidine-1-carboxylic |
| | acid tert-butyl ester |
| 95 | 1-Methyl-2-(6-trifluoromethoxy-benzothiazol-2-ylamino)-1H- |
| | benzoimidazole-5-carboxylic acid piperidin-3-ylamide |
| | hydrochloride |
| 96 | 1-Methyl-2-(6-trifluoromethoxy-benzothiazol-2-ylamino)- |
| | 1H-benzoimidazole-5-carboxylic acid (thiazol-2-ylmethyl)- |
| | amide |
| 97 | 3-{[1-Methyl-2-(6-trifluoromethoxy-benzothiazol-2-ylamino)- |
| | 1H-benzoimidazole-5-carbonyl]-amino}-propionic acid |
| | methyl ester |
| 98 | 3-{[2-(6-Trifluoromethoxy-benzothiazol-2-ylamino)-1-methyl- |
| | 1H-benzimidazole-5-carbonyl]-amino}-propionic acid |
| 99 | 1-Methyl-2-(5-trifluoromethoxy-benzothiazol-2-ylamino)-1H- |
| | benzoimidazole-5-carboxylic acid methyl ester |
| 100 | 1-Methyl-2-(6-trifluoromethoxy-benzothiazol-2-ylamino)-1H- |
| | benzoimidazole-5-carboxylic acid (2-acetylamino-ethyl)-amide |
| 101 | 1-Methyl-2-(6-trifluoromethoxy-benzothiazol-2-ylamino)-1H- |
| | benzoimidazole-5-carboxylic acid (2-methylsulfanyl-ethyl)-amide |
| 102 | 1-Methyl-2-(6-trifluoromethoxy-benzothiazol-2-ylamino)- |
| | 1H-benzoimidazole-5-carboxylic acid (2-methanesulfonyl- |
| | ethyl)-amide |
| 103 | (2-{[1-Methyl-2-(6-trifluoromethoxy-benzothiazol-2-ylamino)- |
| | 1H-benzoimidazole-5-carbonyl]-amino}-ethyl)-carbamic acid |
| | tert-butyl ester |
| 104 | 1-Methyl-2-(6-trifluoromethoxy-benzothiazol-2-ylamino)- |
| | 1H-benzoimidazole-5-carboxylic acid (2-amino-ethyl)- |
| | amide hydrochloride |
| 105 | 1-Methyl-2-(6-trifluoromethoxy-benzothiazol-2-ylamino)-1H- |
| | benzoimidazole-5-carboxylic acid (2-methylamino-ethyl)-amide |
| 106 | 1-Methyl-2-(6-trifluoromethoxy-benzothiazol-2-ylamino)- |
| | 1H-benzimidazole-5-carboxylic acid trimethylhydrazide |
| 107 | 1-Methyl-2-(6-trifluoromethoxy-benzothiazol-2-ylamino)-1H- |
| | benzoimidazole-5-carboxylic acid (2-ethylsulfanyl-ethyl)-amide |
| 108 | 1-Methyl-2-(6-trifluoromethoxy-benzothiazol-2-ylamino)-1H- |
| | benzoimidazole-5-carboxylic acid (3-methylsulfanyl- |
| | propyl)-amide |
| 109 | 1-Methyl-2-(6-trifluoromethoxy-benzothiazol-2-ylamino)-1H- |
| | benzimidazole-5-carboxylic acid (2-ethanesulfonyl-ethyl)-amide |
| 110 | 1-Methyl-2-(6-trifluoromethoxy-benzothiazol-2-ylamino)- |
| | 1H-benzimidazole-5-carboxylic acid (3-methanesulfonyl- |
| | propyl)-amide |
| 111 | 2-(5-Fluoro-benzothiazol-2-ylamino)-1-methyl-1H- |
| | benzoimidazole-5-carboxylic acid methyl ester |
| 112 | 2-(6-Fluoro-benzothiazol-2-ylamino)-1-methyl-1H- |
| | benzoimidazole-5-carboxylic acid methyl ester |
| 113 | 2-(6-Methanesulfonyl-benzothiazol-2-ylamino)-1-methyl-1H- |
| | benzoimidazole-5-carboxylic acid methyl ester |
| 114 | 1-Methyl-2-(6-methyl-benzothiazol-2-ylamino)-1H- |
| | benzoimidazole-5-carboxylic acid methyl ester |
| 115 | 1-Methyl-2-(5-trifluoromethoxy-benzothiazol-2-ylamino)-1H- |
| | benzimidazole-5-carboxylic acid methylamide |
| 116 | 1-Methyl-2-(5-trifluoromethoxy-benzothiazol-2-ylamino)- |
| | 1H-benzimidazole-5-carboxylic acid (2-methoxy-ethyl)-amide |
| 117 | 2-(5-Fluoro-benzothiazol-2-ylamino)-1-methyl-1H- |
| | benzimidazole-5-carboxylic acid |
| 118 | 2-(6-Fluoro-benzothiazol-2-ylamino)-1-methyl-1H- |
| | benzimidazole-5-carboxylic acid |
| 119 | 2-(6-Methanesulfonyl-benzothiazol-2-ylamino)-1-methyl-1H- |
| | benzimidazole-5-carboxylic acid |
| 120 | 1-Methyl-2-(6-methyl-benzothiazol-2-ylamino)-1H- |
| | benzimidazole-5-carboxylic acid |
| 121 | 1-Methyl-2-(6-trifluoromethoxy-benzothiazol-2-ylamino)- |
| | 1H-benzimidazole-5-carboxylic acid (1,1-dioxo-tetrahydro- |
| | 1λ⁶-thiophen-3-yl)-amide |
| 122 | 2-(5-Fluoro-benzothiazol-2-ylamino)-1-methyl-1H- |
| | benzimidazole-5-carboxylic acid methylamide |
| 123 | 2-(5-Fluoro-benzothiazol-2-ylamino)-1-methyl-1H- |
| | benzimidazole-5-carboxylic acid (2-methoxy-ethyl)-amide |
| 124 | 2-(6-Fluoro-benzothiazol-2-ylamino)-1-methyl-1H- |
| | benzimidazole-5-carboxylic acid methylamide |
| 125 | 2-(6-Fluoro-benzothiazol-2-ylamino)-1-methyl-1H- |
| | benzimidazole-5-carboxylic acid (2-methoxy-ethyl)-amide |
| 126 | 2-(6-Methanesulfonyl-benzothiazol-2-ylamino)-1-methyl- |
| | 1H-benzimidazole-5-carboxylic acid methylamide |
| 127 | 2-(6-Methanesulfonyl-benzothiazol-2-ylamino)-1-methyl-1H- |
| | benzimidazole-5-carboxylic acid (2-methoxy-ethyl)-amide |
| 128 | 2-(6-Methyl-benzothiazol-2-ylamino)-1-methyl-1H- |
| | benzimidazole-5-carboxylic acid methylamide |
| 129 | 2-(6-Methyl-benzothiazol-2-ylamino)-1-methyl-1H- |
| | benzimidazole-5-carboxylic acid (2-methoxy-ethyl)-amide |
| 130 | 2-(6-Methanesulfonyl-benzothiazol-2-ylamino)-1-methyl-1H- |
| | benzimidazole-5-carboxylic acid (2-methylsulfanyl-ethyl)-amide |
| 131 | 2-(6-Methanesulfonyl-benzothiazol-2-ylamino)-1-methyl-1H- |
| | benzimidazole-5-carboxylic acid (2-methylsulfonyl-ethyl)-amide |
| 132 | 1-Methyl-2-(6-trifluoromethylsulfanyl-benzothiazol-2-ylamino)- |
| | 1H-benzoimidazole-5-carboxylic acid methyl ester |
| 133 | 2-(6-Chloro-benzothiazol-2-ylamino)-1-methyl-1H- |
| | benzimidazole-5-carboxylic acid dimethylcarbamoylmethyl- |
| | amide |
| 134 | 1-Methyl-2-(6-trifluoromethyl-benzothiazol-2-ylamino)-1H- |
| | benzimidazole-5-carboxylic acid dimethylcarbamoylmethyl- |
| | amide |
| 135 | 1-Methyl-2-(6-trifluoromethoxy-benzothiazol-2-ylamino)-1H- |
| | benzimidazole-5-carboxylic acid (2-dimethylcarbamoyl-ethyl)- |
| | amide |
| 136 | 3-{[1-Methyl-2-(6-trifluoromethoxy-benzothiazol-2- |
| | ylamino)-1H-benzimidazole-5-carbonyl]-amino}-propionic |
| | acid tert-butyl ester |
| 137 | 1-Methyl-2-(6-trifluoromethoxy-benzothiazol-2-ylamino)- |
| | 1H-benzimidazole-5-carboxylic acid [2-(4-methyl- |
| | piperazin-1-yl)-2-oxo-ethyl]-amide |
| 138 | 1-Methyl-2-(6-trifluoromethoxy-benzothiazol-2-ylamino)- |
| | 1H-benzimidazole-5-carboxylic acid (2-morpholin-4- |
| | yl-2-oxo-ethyl)-amide |
| 139 | 1-Methyl-2-(6-trifluoromethoxy-benzothiazol-2-ylamino)-1H- |
| | benzimidazole-5-carboxylic acid methylcarbamoylmethyl-amide |
| 140 | 1-Methyl-2-(6-trifluoromethoxy-benzothiazol-2-ylamino)- |
| | 1H-benzimidazole-5-carboxylic acid diethylcarbamoylmethyl- |
| | amide |
| 141 | 1-Methyl-2-(6-trifluoromethoxy-benzothiazol-2-ylamino)- |
| | 1H-benzimidazole-5-carboxylic acid (2-oxo-2-pyrrolidin-1-yl- |
| | ethyl)-amide |
| 142 | 4-(2-{[1-Methyl-2-(6-trifluoromethoxy-benzothiazol-2- |
| | ylamino)-1H-benzimidazole-5-carbonyl]-amino}-acetyl)- |
| | piperazine-1-carboxylic acid tert-butyl ester |
| 143 | (S)-2-{[1-Methyl-2-(6-trifluoromethoxy-benzothiazol-2- |
| | ylamino)-1H-benzoimidazole-5-carbonyl]-amino}- |
| | propionic acid methyl ester |
| 144 | 1-{[1-Methyl-2-(6-trifluoromethoxy-benzothiazol-2- |
| | ylamino)-1H-benzimidazole-5-carbonyl]-amino}- |
| | cyclopropanecarboxylic acid ethyl ester |
| 145 | 2-Methyl-2-{[1-methyl-2-(6-trifluoromethoxy-benzothiazol-2- |
| | ylamino)-1H-benzimidazole-5-carbonyl]-amino}-propionic |
| | acid methyl ester |
| 146 | (S)-2-{[1-Methyl-2-(6-trifluoromethoxy-benzothiazol-2- |
| | ylamino)-1H-benzoimidazole-5-carbonyl]-amino}- |
| | propionic acid |
| 147 | 1-{[1-Methyl-2-(6-trifluoromethoxy-benzothiazol-2- |
| | ylamino)-1H-benzimidazole-5-carbonyl]-amino}- |
| | cyclopropanecarboxylic acid |
| 148 | 2-Methyl-2-{[1-methyl-2-(6-trifluoromethoxy-benzothiazol- |
| | 2-ylamino)-1H-benzimidazole-5-carbonyl]-amino}- |
| | propionic acid |
| 149 | 1-Methyl-2-(6-trifluoromethoxy-benzothiazol-2-ylamino)-1H- |
| | benzoimidazole-5-carboxylic acid ((S)-1- |
| | dimethylcarbamoyl-ethyl)-amide |
| 150 | 1-Methyl-2-(6-trifluoromethoxy-benzothiazol-2-ylamino)-1H- |
| | benzimidazole-5-carboxylic acid (1-dimethylcarbamoyl- |
| | cyclopropyl)-amide |
| 151 | 1-Methyl-2-(6-trifluoromethoxy-benzothiazol-2-ylamino)-1H- |
| | benzimidazole-5-carboxylic acid (1-dimethylcarbamoyl-1- |
| | methyl-ethyl)-amide |
| 152 | 1-Methyl-2-(6-trifluoromethoxy-benzothiazol-2-ylamino)-1H- |
| | benzimidazole-5-carboxylic acid (2-oxo-2-piperazin-1-yl- |
| | ethyl)-amide hydrochloride |
| 153 | 1-Ethyl-2-(6-trifluoromethoxy-benzothiazol-2-ylamino)-1H- |
| | benzoimidazole-5-carboxylic acid methyl ester |
| 154 | 1-Ethyl-2-(6-trifluoromethoxy-benzothiazol-2-ylamino)-1H- |
| | benzoimidazole-5-carboxylic acid |
| 155 | 1-Ethyl-2-(6-trifluoromethoxy-benzothiazol-2-ylamino)-1H- |
| | benzoimidazole-5-carboxylic acid methylamide |
| 156 | 1-Ethyl-2-(6-trifluoromethoxy-benzothiazol-2-ylamino)-1H- |
| | benzoimidazole-5-carboxylic acid ethylamide |
| 157 | 1-Ethyl-2-(6-trifluoromethoxy-benzothiazol-2-ylamino)-1H- |
| | benzoimidazole-5-carboxylic acid (2-ethoxy-ethyl)-amide |
| 158 | 1-Isopropyl-2-(6-trifluoromethoxy-benzothiazol-2-ylamino)- |
| | 1H benzoimidazole-5-carboxylic acid methyl ester |
| 159 | 1-Isopropyl-2-(6-trifluoromethoxy-benzothiazol-2-ylamino)- |
| | 1H-benzoimidazole-5-carboxylic acid |
| 160 | 1-Isopropyl-2-(6-trifluoromethoxy-benzothiazol-2-ylamino)- |
| | 1H-benzoimidazole-5-carboxylic acid methylamide |
| 161 | 1-Isopropyl-2-(6-trifluoromethoxy-benzothiazol-2-ylamino)- |
| | 1H-benzoimidazole-5-carboxylic acid ethylamide |
| 162 | 1-Isobutyl-2-(6-trifluoromethoxy-benzothiazol-2-ylamino)- |
| | 1H-benzoimidazole-5-carboxylic acid methyl ester |
| 163 | 1-Isobutyl-2-(6-trifluoromethoxy-benzothiazol-2-ylamino)- |
| | 1H-benzoimidazole-5-carboxylic acid |
| 164 | 1-Isobutyl-2-(6-trifluoromethoxy-benzothiazol-2-ylamino)- |
| | 1H-benzoimidazole-5-carboxylic acid methylamide |
| 165 | 1-Isobutyl-2-(6-trifluoromethoxy-benzothiazol-2-ylamino)- |
| | 1H-benzoimidazole-5-carboxylic acid ethylamide |
| 166 | 1-(2-Methoxy-ethyl)-2-(6-trifluoromethoxy-benzothiazol-2- |
| | ylamino)-1H-benzoimidazole-5-carboxylic acid |
| 167 | 1-(2-Methoxy-ethyl)-2-(6-trifluoromethoxy-benzothiazol-2- |
| | ylamino)-1H-benzoimidazole-5-carboxylic acid methylamide |
| 168 | 1-(2-Methoxy-ethyl)-2-(6-trifluoromethoxy-benzothiazol-2- |
| | ylamino)-1H-benzoimidazole-5-carboxylic acid |
| | (2-methoxy-ethyl)-amide |
| 169 | 1-(2-Methoxy-ethyl)-2-(6-trifluoromethoxy-benzothiazol-2- |
| | ylamino)-1H-benzoimidazole-5-carboxylic acid |
| | (2-ethoxy-ethyl)-amide |
| 170 | 1-(2-Fluoro-ethyl)-2-(6-trifluoromethoxy-benzothiazol-2- |
| | ylamino)-1H-benzoimidazole-5-carboxylic acid methylamide |
| 171 | 1-(2-Fluoro-ethyl)-2-(6-trifluoromethoxy-benzothiazol-2- |
| | ylamino)-1H-benzoimidazole-5-carboxylic acid |
| | (2-methoxy-ethyl)-amide |
| 172 | 1-(2-Fluoro-ethyl)-2-(6-trifluoromethoxy-benzothiazol-2- |
| | ylamino)-1H-benzoimidazole-5-carboxylic acid |
| | (2-ethoxy-ethyl)-amide |
| 173 | 1-(2-Amino-ethyl)-2-(6-trifluoromethoxy-benzothiazol-2- |
| | ylamino)-1H-benzoimidazole-5-carboxylic acid |
| | methylamide hydrochloride |
| 174 | 2-(6-Chloro-benzothiazol-2-ylamino)-1-ethyl-1H- |
| | benzoimidazole-5-carboxylic acid methylamide |
| 175 | 2-(6-Chloro-benzothiazol-2-ylamino)-1-ethyl-1H- |
| | benzoimidazole-5-carboxylic acid ethylamide |
| 176 | 2-(6-Chloro-benzothiazol-2-ylamino)-1-ethyl-1H- |
| | benzoimidazole-5-carboxylic acid (2-fluoro-ethyl)-amide |
| 177 | 2-(6-Chloro-benzothiazol-2-ylamino)-1-ethyl-1H- |
| | benzoimidazole-5-carboxylic acid (2-methoxy-ethyl)-amide |
| 178 | 2-(6-Chloro-benzothiazol-2-ylamino)-1-ethyl-1H- |
| | benzoimidazole-5-carboxylic acid (2-methoxy-2-methyl- |
| | propyl)-amide |
| 179 | 2-(6-Chloro-benzothiazol-2-ylamino)-1-ethyl-1H- |
| | benzoimidazole-5-carboxylic acid (2-ethoxy-ethyl)-amide |
| 180 | 1-Ethyl-2-(6-trifluoromethyl-benzothiazol-2-ylamino)-1H- |
| | benzoimidazole-5-carboxylic acid methylamide |
| 181 | 1-Ethyl-2-(6-trifluoromethyl-benzothiazol-2-ylamino)-1H- |
| | benzoimidazole-5-carboxylic acid ethylamide |
| 182 | 1-Ethyl-2-(6-trifluoromethyl-benzothiazol-2-ylamino)-1H- |
| | benzoimidazole-5-carboxylic acid (2-methoxy-ethyl)-amide |
| 183 | 1-Ethyl-2-(6-trifluoromethyl-benzothiazol-2-ylamino)-1H- |
| | benzoimidazole-5-carboxylic acid (2-ethoxy-ethyl)-amide |
| 184 | 1-Ethyl-2-(6-trifluoromethyl-benzothiazol-2-ylamino)-1H- |
| | benzoimidazole-5-carboxylic acid (2-methoxy-2-methyl-propyl)- |
| | amide |
| 185 | 1-Ethyl-2-(6-trifluoromethyl-benzothiazol-2-ylamino)-1H- |
| | benzoimidazole-5-carboxylic acid (2-methylsulfanyl-ethyl)- |
| | amide |
| 186 | 1-Ethyl-2-(6-trifluoromethoxy-benzothiazol-2-ylamino)-1H- |
| | benzoimidazole-5-carboxylic acid dimethylcarbamoylmethyl- |
| | amide |
| 187 | 1-Ethyl-2-(6-trifluoromethyl-benzothiazol-2-ylamino)-1H- |
| | benzoimidazole-5-carboxylic acid dimethylcarbamoylmethyl- |
| | amide |
| 188 | 1-(2-Methoxy-ethyl)-2-(6-trifluoromethoxy-benzothiazol-2- |
| | ylamino)-1H-benzoimidazole-5-carboxylic acid dimethyl- |
| | carbamoylmethyl-amide |
| 189 | 1-(2-Methoxy-ethyl)-2-(6-trifluoromethoxy-benzothiazol-2- |
| | ylamino)-1H-benzoimidazole-5-carboxylic acid [2-(4-methyl- |
| | piperazin-1-yl)-2-oxo-ethyl]-amide |
| 190 | 1-Ethyl-2-(6-trifluoromethoxy-benzothiazol-2-ylamino)-1H- |
| | benzoimidazole-5-carboxylic acid [2-(4-methyl-piperazin-1- |
| | yl)-2-oxo-ethyl]-amide |
| 191 | 1-Ethyl-2-[6-(pyridin-3-yloxy)-benzothiazol-2-ylamino]-1H- |
| | benzoimidazole-5-carboxylic acid (2-methoxy-ethyl)-amide |
| 192 | 1-Methyl-2-(6-trifluoromethoxy-benzothiazol-2-ylamino)-1H- |
| | benzoimidazole-5-carboxylic acid [2-(4-hydroxy-piperidin-1-yl)- |
| | ethyl]-amide |
| 193 | 1-Methyl-2-(6-trifluoromethoxy-benzothiazol-2-ylamino)-1H- |
| | benzoimidazole-5-carboxylic acid [2-(3-hydroxy-piperidin-1-yl)- |
| | ethyl]-amide |
| 194 | 1-Methyl-2-(6-trifluoromethoxy-benzothiazol-2-ylamino)-1H- |
| | benzoimidazole-5-carbonitrile |
| 195 | 1-Methyl-2-(6-trifluoromethoxy-benzothiazol-2-ylamino)-1H- |
| | benzoimidazole-6-carbonitrile |
| 196 | [5-(1H-Imidazol-2-yl)-1-methyl-1H-benzimidazol-2-yl]-(6- |
| | trifluoromethoxy-benzothiazol-2-yl)-amine |
| 197 | [1-Methyl-6-(1H-1,2,4-triazol-3-yl)-1H-benzimidazol-2-yl]- |
| | (6-trifluoromethoxy-benzothiazol-2-yl)-amine |
| 198 | [1-Methyl-6-(5-methyl-1H-1,2,4-triazol-3-yl)-1H-benzimidazol- |
| | 2-yl]-(5-trifluoromethoxy-benzothiazol-2-yl)-amine |
| 199 | (1-Ethyl-5-trifluoromethanesulfonyl-1H-benzoimidazol-2-yl)-(6- |
| | trifluoromethoxy-benzothiazol-2-yl)-amine |
| 200 | 1-[1-Methyl-2-(6-trifluoromethoxy-benzothiazol-2-ylamino)-1H- |
| | benzoimidazol-5-yl]-ethanone |
| 201 | (5-Methanesulfonyl-1-methyl-1H-benzoimidazol-2-yl)-(6- |
| | trifluoromethoxy-benzothiazol-2-yl)-amine |
| 202 | 2-[1-Methyl-2-(6-trifluoromethoxy-benzothiazol-2-ylamino)-1H- |
| | benzoimidazol-6-yl]-acetamide |
| 203 | 2-(6-Chloro-benzothiazol-2-ylamino)-1-methyl-1H- |
| | benzoimidazole-5-carboxylic acid ((R)-2-hydroxy-propyl)-amide |
| 204 | 2-(6-Chloro-benzothiazol-2-ylamino)-1-methyl-1H- |
| | benzoimidazole-5-carboxylic acid ((S)-2-hydroxy-propyl)-amide |
| 205 | 1-Methyl-2-(6-trifluoromethyl-benzothiazol-2-ylamino)-1H- |
| | benzoimidazole-5-carboxylic acid ((R)-2-hydroxy-propyl)-amide |
| 206 | 1-Methyl-2-(6-trifluoromethyl-benzothiazol-2-ylamino)-1H- |
| | benzoimidazole-5-carboxylic acid ((S)-2-hydroxy-propyl)-amide |
| 207 | 2-(6-Chloro-benzothiazol-2-ylamino)-1-methyl-1H- |
| | benzoimidazole-5-carboxylic acid (2-methoxy-2-methyl-propyl)- |
| | amide |
| 208 | 1-Methyl-2-(6-trifluoromethyl-benzothiazol-2-ylamino)-1H- |
| | benzoimidazole-5-carboxylic acid (2-methoxy-2-methyl-propyl)- |
| | amide |
| 209 | 2-(6-Chloro-benzothiazol-2-ylamino)-1-methyl-1H- |
| | benzoimidazole-5-carboxylic acid (2-fluoro-ethyl)-amide |
| 210 | 1-Methyl-2-(6-trifluoromethyl-benzothiazol-2-ylamino)-1H- |
| | benzoimidazole-5-carboxylic acid (2-fluoro-ethyl)-amide |
| 211 | 1-Methyl-2-(6-trifluoromethoxy-benzothiazol-2-ylamino)-1H- |
| | benzoimidazole-5-carboxylic acid cyanomethyl-amide |
| 212 | 1-Methyl-2-(6-trifluoromethoxy-benzothiazol-2-ylamino)-1H- |
| | benzoimidazole-5-carboxylic acid (2-cyano-ethyl)-amide |
| 213 | 2-(6-Chloro-benzothiazol-2-ylamino)-1-methyl-1H- |
| | benzoimidazole-5-carboxylic acid (2-cyano-ethyl)-amide |
| 214 | 2-(6-Chloro-benzothiazol-2-ylamino)-1-methyl-1H- |
| | benzoimidazole-5-carboxylic acid (3-hydroxy-propyl)-amide |
| 215 | 2-(6-Chloro-benzothiazol-2-ylamino)-1-methyl-1H- |
| | benzoimidazole-5-carboxylic acid (3-hydroxy-butyl)-amide |
| 216 | 1-Methyl-2-(6-trifluoromethyl-benzothiazol-2-ylamino)-1H- |
| | benzoimidazole-5-carboxylic acid (3-hydroxy-butyl)-amide |
| 217 | 1-Methyl-2-(6-trifluoromethoxy-benzothiazol-2-ylamino)-1H- |
| | benzoimidazole-5-carboxylic acid (3-hydroxy-2,2-dimethyl- |
| | propyl)-amide |
| 218 | 2-(6-Chloro-benzothiazol-2-ylamino)-1-methyl-1H- |
| | benzoimidazole-5-carboxylic acid (3-hydroxy-2,2-dimethyl- |
| | propyl)-amide |
| 219 | 1-Methyl-2-(6-trifluoromethoxy-benzothiazol-2-ylamino)-1H- |
| | benzoimidazole-5-carboxylic acid (4-hydroxy-butyl)-amide |
| 220 | 2-(6-Chloro-1H-benzoimidazol-2-ylamino)-1-methyl-1H- |
| | benzoimidazole-5-carboxylic acid (4-hydroxy-butyl)-amide |
| 221 | 1-Methyl-2-(6-trifluoromethyl-benzothiazol-2-ylamino)-1H- |
| | benzoimidazole-5-carboxylic acid (4-hydroxy-butyl)-amide |
| 222 | 6-Fluoro-1-methyl-2-(6-trifluoromethoxy-benzothiazol-2- |
| | ylamino)-1H-benzoimidazole-5-carboxylic acid (4-hydroxy- |
| | butyl)-amide |
| 223 | 1-Methyl-2-(6-trifluoromethoxy-benzothiazol-2-ylamino)-1H- |
| | benzoimidazole-5-carboxylic acid ((R)-4-hydroxy-3-methyl- |
| | butyl)-amide |
| 224 | 2-(6-Chloro-benzothiazol-2-ylamino)-1-methyl-1H- |
| | benzoimidazole-5-carboxylic acid ((R)-4-hydroxy-3-methyl- |
| | butyl)-amide |
| 225 | 2-(6-Chloro-benzothiazol-2-ylamino)-1-methyl-1H- |
| | benzoimidazole-5-carboxylic acid (trans-4-hydroxy-cyclohexyl)- |
| | amide |
| 226 | 1-Methyl-2-(6-trifluoromethoxy-benzothiazol-2-ylamino)-1H- |
| | benzoimidazole-5-carboxylic acid (5-hydroxy-pentyl)-amide |
| 227 | 2-(6-Chloro-benzothiazol-2-ylamino)-1-methyl-1H- |
| | benzoimidazole-5-carboxylic acid (5-hydroxy-pentyl)-amide |
| 228 | 1-Methyl-2-(6-trifluoromethyl-benzothiazol-2-ylamino)-1H- |
| | benzoimidazole-5-carboxylic acid (5-hydroxy-pentyl)-amide |
| 229 | 1-Methyl-2-(6-trifluoromethoxy-benzothiazol-2-ylamino)-1H- |
| | benzoimidazole-5-carboxylic acid (5-hydroxy-4,4-dimethyl- |
| | pentyl)-amide |
| 230 | 1-Methyl-2-(6-trifluoromethoxy-benzothiazol-2-ylamino)-1H- |
| | benzoimidazole-5-carboxylic acid 4-hydroxy-benzylamide |
| 231 | 1-Methyl-2-(6-trifluoromethoxy-benzothiazol-2-ylamino)-1H- |
| | benzoimidazole-5-carboxylic acid 3-hydroxy-4-methoxy- |
| | benzylamide |
| 232 | 1-Methyl-2-(6-trifluoromethoxy-benzothiazol-2-ylamino)-1H- |
| | benzoimidazole-5-carboxylic acid (trans-4-hydroxy- |
| | cyclohexylmethyl)-amide |
| 233 | 2-(6-Chloro-benzothiazol-2-ylamino)-1-methyl-1H- |
| | benzoimidazole-5-carboxylic acid (trans-4-hydroxy- |
| | cyclohexylmethyl)-amide |
| 234 | 1-Methyl-2-(6-trifluoromethyl-benzothiazol-2-ylamino)-1H- |
| | benzoimidazole-5-carboxylic acid (trans-4-hydroxy- |
| | cyclohexylmethyl)-amide |
| 235 | 2-(6-Chloro-benzothiazol-2-ylamino)-1-methyl-1H- |
| | benzoimidazole-5-carboxylic acid [2-(2-hydroxy-ethoxy)- |
| | ethyl]-amide |
| 236 | 1-Methyl-2-(6-trifluoromethyl-benzothiazol-2-ylamino)-1H- |
| | benzoimidazole-5-carboxylic acid [2-(2-hydroxy-ethoxy)- |
| | ethyl]-amide |
| 237 | 6-Fluoro-1-methyl-2-(6-trifluoromethoxy-benzothiazol-2- |
| | ylamino)-1H-benzoimidazole-5-carboxylic acid [2-(2-hydroxy- |
| | ethoxy)-ethyl]-amide |
| 238 | 3-Methyl-2-(6-trifluoromethoxy-benzothiazol-2-ylamino)-3H- |
| | imidazo[4,5-b]pyridine-6-carboxylic acid [2-(2-hydroxy-ethoxy)- |
| | ethyl]-amide |
| 239 | 1-(2-Methylamino-ethyl)-2-(6-trifluoromethoxy-benzothiazol-2 |
| | ylamino)-1H-benzoimidazole-5-carboxylic acid[2-(2-hydroxy- |
| | ethoxy)-ethyl]-amide hydrochloride |
| 240 | 2-(6-Chloro-benzothiazol-2-ylamino)-1-(2-methylamino-ethyl)- |
| | 1H-benzoimidazole-5-carboxylic acid [2-(2-hydroxy-ethoxy)- |
| | ethyl]-amide hydrochloride |
| 241 | 1-(2-Methoxy-ethyl)-2-(6-trifluoromethoxy-benzothiazol-2- |
| | ylamino)-1H-benzoimidazole-5-carboxylic acid [2-(2-hydroxy- |
| | ethoxy)-ethyl]-amide |
| 242 | 1-Methyl-2-(6-trifluoromethoxy-benzothiazol-2-ylamino)-1H- |
| | benzoimidazole-5-carboxylic acid [2-((R)-2-hydroxy-1- |
| | methyl-ethoxy)-ethyl]-amide |
| 243 | 2-(6-Chloro-benzothiazol-2-ylamino)-1-methyl-1H- |
| | benzoimidazole-5-carboxylic acid [2-((R)-2-hydroxy-1-methyl- |
| | ethoxy)-ethyl]-amide |
| 244 | 1-Methyl-2-(6-trifluoromethoxy-benzothiazol-2-ylamino)-1H- |
| | benzoimidazole-5-carboxylic acid [2-(2-hydroxy-propoxy)- |
| | ethyl]-amide |
| 245 | 1-Methyl-2-(6-trifluoromethoxy-benzothiazol-2-ylamino)-1H- |
| | benzoimidazole-5-carboxylic acid [2-(2-hydroxy-2-methyl- |
| | propoxy)-ethyl]-amide |
| 246 | 1-Methyl-2-(6-trifluoromethoxy-benzothiazol-2-ylamino)-1H- |
| | benzoimidazole-5-carboxylic acid [2-(3-hydroxy-propoxy)- |
| | ethyl]-amide |
| 247 | 1-Methyl-2-(6-trifluoromethoxy-benzothiazol-2-ylamino)-1H- |
| | benzoimidazole-5-carboxylic acid [2-(3-fluoro-propoxy)-ethyl]- |
| | amide |
| 248 | 2-(6-Chloro-benzothiazol-2-ylamino)-1-methyl-1H- |
| | benzoimidazole-5-carboxylic acid [2-(3-hydroxy-propoxy)- |
| | ethyl]-amide |
| 249 | 2-(6-Chloro-benzothiazol-2-ylamino)-1-methyl-1H- |
| | benzoimidazole-5-carboxylic acid [2-(3-fluoro-propoxy)- |
| | ethyl]-amide |
| 250 | 1-Methyl-2-(6-trifluoromethoxy-benzothiazol-2-ylamino)- |
| | 1H-benzoimidazole-5-carboxylic acid [3-(2-hydroxy-ethoxy)- |
| | propyl]-amide |
| 251 | 1-Methyl-2-(6-trifluoromethoxy-benzothiazol-2-ylamino)-1H- |
| | benzoimidazole-5-carboxylic acid [2-(4-hydroxy-phenyl)-ethyl]- |
| | amide |
| 252 | 1-Methyl-2-(6-trifluoromethoxy-benzothiazol-2-ylamino)-1H- |
| | benzoimidazole-5-carboxylic acid [2-(3-hydroxy-phenyl)-ethyl]- |
| | amide |
| 253 | 1-Methyl-2-(6-trifluoromethoxy-benzothiazol-2-ylamino)-1H- |
| | benzoimidazole-5-carboxylic acid [2-(4-hydroxy-cyclohexyl)- |
| | ethyl]-amide |
| 254 | 1-Methyl-2-(6-trifluoromethoxy-benzothiazol-2-ylamino)-1H- |
| | benzoimidazole-5-carboxylic acid (trans-4-hydroxymethyl- |
| | cyclohexylmethyl)-amide |
| 255 | 2-(6-Chloro-benzothiazol-2-ylamino)-1-methyl-1H- |
| | benzoimidazole-5-carboxylic acid (trans-4-hydroxymethyl- |
| | cyclohexylmethyl)-amide |
| 256 | 1-Methyl-2-(6-trifluoromethoxy-benzothiazol-2-ylamino)- |
| | 1H-benzoimidazole-5-carboxylic acid {2-[2-(2-hydroxy- |
| | ethoxy)-ethoxy]-ethyl}-amide |
| 257 | 1-Methyl-2-(6-trifluoromethyl-benzothiazol-2-ylamino)-1H- |
| | benzoimidazole-5-carboxylic acid [2-(2-fluoro-ethoxy)-ethyl]- |
| | amide |
| 258 | 1-Methyl-2-(6-trifluoromethoxy-benzothiazol-2-ylamino)-1H- |
| | benzoimidazole-5-carboxylic acid [2-(2,2-difluoro-ethoxy)- |
| | ethyl]-amide |
| 259 | 2-(6-Chloro-benzothiazol-2-ylamino)-1-methyl-1H- |
| | benzoimidazole-5-carboxylic acid [2-(2,2-difluoro-ethoxy)- |
| | ethyl]-amide |
| 260 | 1-Methyl-2-(6-trifluoromethoxy-benzothiazol-2-ylamino)-1H- |
| | benzoimidazole-5-carboxylic acid [2-(2-methoxy-ethoxy)-ethyl]- |
| | amide |
| 261 | 2-(6-Chloro-benzothiazol-2-ylamino)-1-methyl-1H- |
| | benzoimidazole-5-carboxylic acid [2-(2-methoxy-ethoxy)-ethyl]- |
| | amide |
| 262 | 1-Methyl-2-(6-trifluoromethyl-benzothiazol-2-ylamino)-1H- |
| | benzoimidazole-5-carboxylic acid [2-(2-methoxy-ethoxy)-ethyl]- |
| | amide |
| 263 | 1-Methyl-2-(6-trifluoromethoxy-benzothiazol-2-ylamino)-1H- |
| | benzoimidazole-5-carboxylic acid [2-(tetrahydro-pyran-2-yl)- |
| | ethyl]-amide |
| 264 | 1-Methyl-2-(6-trifluoromethoxy-benzothiazol-2-ylamino)-1H- |
| | benzoimidazole-5-carboxylic acid [2-(tetrahydro-pyran-4-yl)- |
| | ethyl]-amide |
| 265 | 1-Methyl-2-(6-trifluoromethoxy-benzothiazol-2-ylamino)-1H- |
| | benzoimidazole-5-carboxylic acid [2-(2-cyano-ethoxy)-ethyl]- |
| | amide |
| 266 | 2-(6-Chloro-benzothiazol-2-ylamino)-1-methyl-1H- |
| | benzoimidazole-5-carboxylic acid [2-(2-cyano-ethoxy)-ethyl]- |
| | amide |
| 267 | 1-Methyl-2-(6-trifluoromethoxy-benzothiazol-2-ylamino)-1H- |
| | benzoimidazole-5-carboxylic acid (2-carbamoylmethoxy-ethyl)- |
| | amide |
| 268 | 1-Methyl-2-(6-trifluoromethoxy-benzothiazol-2-ylamino)-1H- |
| | benzoimidazole-5-carboxylic acid [2-(2-amino-ethoxy)-ethyl]- |
| | amide |
| 269 | 2-(6-Chloro-benzothiazol-2-ylamino)-1-methyl-1H- |
| | benzoimidazole-5-carboxylic acid [2-(2-amino-ethoxy)-ethyl]- |
| | amide |
| 270 | 2-(4-Chloro-benzothiazol-2-ylamino)-1-methyl-1H |
| | benzoimidazole-5-carboxylic acid [2-(2-amino-ethoxy)-ethyl]- |
| | amide |
| 271 | 1-Methyl-2-(6-trifluoromethoxy-benzothiazol-2-ylamino)-1H- |
| | benzoimidazole-5-carboxylic acid [2-(2-methylamino-ethoxy)- |
| | ethyl]-amide hydrochloride |
| 272 | 2-(6-Chloro-benzothiazol-2-ylamino)-1-methyl-1H- |
| | benzoimidazole-5-carboxylic acid [2-(2-methylamino-ethoxy)- |
| | ethyl]-amide hydrochloride |
| 273 | 1-Methyl-2-(6-trifluoromethoxy-benzothiazol-2-ylamino)-1H- |
| | benzoimidazole-5-carboxylic acid [2-(2-dimethylamino-ethoxy)- |
| | ethyl]-amide |
| 274 | 2-(6-Chloro-benzothiazol-2-ylamino)-1-methyl-1H- |
| | benzoimidazole-5-carboxylic acid [2-(2-dimethylamino-ethoxy)- |
| | ethyl]-amide |
| 275 | 1-Methyl-2-(6-trifluoromethoxy-benzothiazol-2-ylamino)-1H- |
| | benzoimidazole-5-carboxylic acid [2-(2-acetylamino-ethoxy)- |
| | ethyl]-amide |
| 276 | 1-Methyl-2-(6-trifluoromethoxy-benzothiazol-2-ylamino)-1H- |
| | benzoimidazole-5-carboxylic acid [2-(2-methanesulfonylamino- |
| | ethoxy)-ethyl]-amide |
| 277 | 2-(6-Chloro-benzothiazol-2-ylamino)-1-methyl-1H- |
| | benzoimidazole-5-carboxylic acid (2-ethanesulfonyl-ethyl)- |
| | amide |
| 278 | 2-(6-Chloro-benzothiazol-2-ylamino)-1-methyl-1H- |
| | benzoimidazole-5-carboxylic acid [2-(2-hydroxy- |
| | ethanesulfonyl)-ethyl]-amide |
| 279 | 2-(6-Chloro-benzothiazol-2-ylamino)-1-methyl-1H- |
| | benzoimidazole-5-carboxylic acid [2-(2-fluoro-ethylamino)- |
| | ethyl]-amide hydrochloride |
| 280 | 1-Methyl-2-(6-trifluoromethoxy-benzothiazol-2-ylamino)-1H- |
| | benzoimidazole-5-carboxylic acid ((S)-2,3-dihydroxy-propyl)- |
| | amide |
| 281 | 1-Methyl-2-(6-trifluoromethoxy-benzothiazol-2-ylamino)-1H- |
| | benzoimidazole-5-carboxylic acid ((R)-2,3-dihydroxy-propyl)- |
| | amide |
| 282 | 1-Methyl-2-(6-trifluoromethoxy-benzothiazol-2-ylamino)-1H- |
| | benzoimidazole-5-carboxylic acid ((1R,2S,3R,4R)-2,3-dihydroxy- |
| | 4-hydroxymethyl-cyclopentyl)-amide |
| 283 | 1-Methyl-2-(6-trifluoromethoxy-benzothiazol-2-ylamino)-1H- |
| | benzoimidazole-5-carboxylic acid ((2S,3R,4R,5S,6R)-2,4,5- |
| | trihydroxy-6-hydroxymethyl-tetrahydro-pyran-3-yl)-amide |
| 284 | 1-Methyl-2-(6-trifluoromethoxy-benzothiazol-2-ylamino)-1H- |
| | benzoimidazole-5-carboxylic acid bis-(2-hydroxy-ethyl)-amide |
| 285 | 3-Methyl-2-(6-trifluoromethoxy-benzothiazol-2-ylamino)-3H- |
| | benzoimidazole-5-carboxylic acid (4-hydroxy-butyl)-amide |
| 286 | 3-Methyl-2-(6-trifluoromethoxy-benzothiazol-2-ylamino)-3H- |
| | benzoimidazole-5-carboxylic acid [2-(2-hydroxy-ethoxy)-ethyl]- |
| | amide |
| 287 | 2-(6-Chloro-benzothiazol-2-ylamino)-3-methyl-3H- |
| | benzoimidazole-5-carboxylic acid (4-hydroxy-butyl)-amide |
| 288 | 2-(6-Chloro-benzothiazol-2-ylamino)-3-methyl-3H- |
| | benzoimidazole-5-carboxylic acid [2-(2-hydroxy-ethoxy)-ethyl]- |
| | amide |
| 289 | 1-Methyl-2-(6-trifluoromethoxy-benzothiazol-2-ylamino)-1H- |
| | benzoimidazole-5-carboxylic acid pyrrolidin-3-ylamide |
| | hydrochloride |
| 290 | 2-(6-Chloro-benzothiazol-2-ylamino)-1-methyl-1H- |
| | benzoimidazole-5-carboxylic acid (R)-pyrrolidin-3-ylamide |
| | hydrochloride |
| 291 | 2-(6-Chloro-benzothiazol-2-ylamino)-1-methyl-1H- |
| | benzoimidazole-5-carboxylic acid (S)-pyrrolidin-3-ylamide |
| | hydrochloride |
| 292 | 2-(6-Chloro-benzothiazol-2-ylamino)-1-methyl-1H- |
| | benzoimidazole-5-carboxylic acid [(R)-1-(2-hydroxy-ethyl)- |
| | pyrrolidin-3-yl]-amide |
| 293 | 2-(6-Chloro-benzothiazol-2-ylamino)-1-methyl-1H- |
| | benzoimidazole-5-carboxylic acid [(S)-1-(2-hydroxy-ethyl)- |
| | pyrrolidin-3-yl]-amide |
| 294 | 1-Methyl-2-(6-trifluoromethoxy-benzothiazol-2-ylamino)-1H- |
| | benzoimidazole-5-carboxylic acid [1-((R)-2-hydroxy-propyl)- |
| | pyrrolidin-3-yl]-amide |
| 295 | 2-(6-Chloro-benzothiazol-2-ylamino)-1-methyl-1H- |
| | benzoimidazole-5-carboxylic acid [(R)-1-((S)-2-hydroxy- |
| | propionyl)-pyrrolidin-3-yl]-amide |
| 296 | 2-(6-Chloro-benzothiazol-2-ylamino)-1-methyl-1H- |
| | benzoimidazole-5-carboxylic acid [(R)-1-((R)-2-hydroxy-propyl)- |
| | pyrrolidin-3-yl]-amide |
| 297 | 1-Methyl-2-(6-trifluoromethyl-benzothiazol-2-ylamino)-1H- |
| | benzoimidazole-5-carboxylic acid [(R)-1-((R)-2-hydroxy-propyl)- |
| | pyrrolidin-3-yl]-amide |
| 298 | 1-Methyl-2-(6-trifluoromethoxy-benzothiazol-2-ylamino)-1H- |
| | benzoimidazole-5-carboxylic acid [1-(2-hydroxy-2-methyl- |
| | propyl)-pyrrolidin-3-yl]-amide |
| 299 | 3-(3-{[2-(6-Chloro-benzothiazol-2-ylamino)-1-methyl-1H- |
| | benzoimidazole-5-carbonyl]-amino}-pyrrolidin-1-yl)-propionic |
| | acid |
| 300 | 2-(6-Chloro-benzothiazol-2-ylamino)-1-methyl-1H- |
| | benzoimidazole-5-carboxylic acid [1-(2-methanesulfonylamino- |
| | ethyl)-pyrrolidin-3-yl]-amide |
| 301 | 2-(6-Chloro-benzothiazol-2-ylamino)-1-(2-methoxy-ethyl)-1H- |
| | benzoimidazole-5-carboxylic acid [1-(2-hydroxy-ethyl)- |
| | piperidin-4-yl]-amide |
| 302 | 2-(6-Chloro-benzothiazol-2-ylamino)-1-methyl-1H- |
| | benzoimidazole-5-carboxylic acid (piperidin-4-ylmethyl)-amide |
| | hydrochloride |
| 303 | 2-(6-Chloro-benzothiazol-2-ylamino)-1-methyl-1H- |
| | benzoimidazole-5-carboxylic acid [1-(2-hydroxy-ethyl)- |
| | piperidin-4-ylmethyl]-amide |
| 304 | 2-(6-Chloro-benzothiazol-2-ylamino)-1-(2-methoxy-ethyl)-1H- |
| | benzoimidazole-5-carboxylic acid [1-(2-hydroxy-ethyl)- |
| | piperidin-4-ylmethyl]-amide |
| 305 | [4-(2-Hydroxy-ethyl)-piperazin-1-yl]-[1-methyl-2-(6- |
| | trifluoromethoxy-benzothiazol-2-ylamino)-1H-benzoimidazol- |
| | 5-yl]-methanone |
| 306 | [2-(6-Chloro-benzothiazol-2-ylamino)-1-methyl-1H- |
| | benzoimidazol-5-yl]-[4-(2-hydroxy-ethyl)-piperazin-1-yl]- |
| | methanone |
| 307 | [4-(3-Hydroxy-propyl)-piperidin-1-yl]-[1-methyl-2-(6- |
| | trifluoromethoxy-benzothiazol-2-ylamino)-1H benzoimidazol-5- |
| | yl]-methanone |
| 308 | 2-(6-Chloro-benzothiazol-2-ylamino)-1-methyl-1H- |
| | benzoimidazole-5-carboxylic acid [(R)-1-(2-dimethylamino- |
| | acetyl)-pyrrolidin-3-yl]-amide |
| 309 | 2-(6-Chloro-benzothiazol-2-ylamino)-1-methyl-1H- |
| | benzoimidazole-5-carboxylic acid (R)-piperidin-3-ylamide |
| | hydrochloride |
| 310 | 2-(6-Chloro-benzothiazol-2-ylamino)-1-methyl-1H- |
| | benzoimidazole-5-carboxylic acid (S)-piperidin-3-ylamide |
| | hydrochloride |
| 311 | 2-(6-Chloro-benzothiazol-2-ylamino)-1-methyl-1H- |
| | benzoimidazole-5-carboxylic acid [(R)-1-(2-dimethylamino- |
| | acetyl)-piperidin-3-yl]-amide |
| 312 | 2-(6-Chloro-benzothiazol-2-ylamino)-1-methyl-1H- |
| | benzoimidazole-5-carboxylic acid [(S)-1-(2-dimethylamino- |
| | acetyl)-piperidin-3-yl]-amide |
| 313 | 2-(6-Chloro-benzothiazol-2-ylamino)-1-methyl-1H- |
| | benzoimidazole-5-carboxylic acid [1-(2-dimethylamino- |
| | acetyl)-piperidin-4-yl]-amide |
| 314 | 2-(6-Chloro-benzothiazol-2-ylamino)-1-methyl-1H- |
| | benzoimidazole-5-carboxylic acid [1-(2-dimethylamino-acetyl)- |
| | piperidin-4-ylmethyl]-amide |
| 315 | 2-(6-Chloro-benzothiazol-2-ylamino)-1-(2-methoxy-ethyl)-1H- |
| | benzoimidazole-5-carboxylic acid [1-(2-dimethylamino-acetyl)- |
| | piperidin-4-ylmethyl]-amide |
| 316 | 2-(6-Chloro-benzothiazol-2-ylamino)-1-methyl-1H |
| | benzoimidazole-5-carboxylic acid ((R)-1-methyl-pyrrolidin-3- |
| | yl)-amide |
| 317 | 2-(6-Chloro-benzothiazol-2 ylamino)-1-methyl-1H- |
| | benzoimidazole-5-carboxylic acid ((S)-1-methyl-pyrrolidin-3-yl)- |
| | amide |
| 318 | 2-(6-Chloro-benzothiazol-2-ylamino)-1-methyl-1H- |
| | benzoimidazole-5-carboxylic acid (1-methyl-piperidin-2- |
| | ylmethyl)-amide |
| 319 | 2-(6-Chloro-benzothiazol-2-ylamino)-1-methyl-1H- |
| | benzoimidazole-5-carboxylic acid (1-methyl-piperidin-4-yl)- |
| | amide |
| 320 | 2-(6-Chloro-benzothiazol-2-ylamino)-1-methyl-1H- |
| | benzoimidazole-5-carboxylic acid (1-methanesulfonyl- |
| | piperidin-4-yl)-amide |
| 321 | 2-(6-Chloro-benzothiazol-2-ylamino)-1-methyl-1H- |
| | benzoimidazole-5-carboxylic acid morpholin-4-ylamide |
| 322 | 1-Methyl-2-(6-trifluoromethoxy-benzothiazol-2-ylamino)-1H- |
| | benzoimidazole-5-carboxylic acid (2-methanesulfonylamino- |
| | ethyl)-amide |
| 323 | 1-Methyl-2-(6-trifluoromethoxy-benzothiazol-2-ylamino)-1H- |
| | benzoimidazole-5-carboxylic acid [2-(2-dimethylamino- |
| | acetylamino)-ethyl]-amide |
| 324 | 2-(6-Chloro-benzothiazol-2-ylamino)-1-methyl-1H- |
| | benzoimidazole-5-carboxylic acid [2-(2-dimethylamino- |
| | acetylamino)-ethyl]-amide |
| 325 | 1-Methyl-2-(6-trifluoromethoxy-benzothiazol-2-ylamino)-1H- |
| | benzoimidazole-5-carboxylic acid [2-(2-hydroxy-acetylamino)- |
| | ethyl]-amide |
| 326 | 1-Methyl-2-(6-trifluoromethoxy-benzothiazol-2-ylamino)-1H- |
| | benzoimidazole-5-carboxylic acid [2-((S)-2-hydroxy- |
| | propionylamino)-ethyl]-amide |
| 327 | 1-Methyl-2-(6-trifluoromethoxy-benzothiazol-2-ylamino)-1H- |
| | benzoimidazole-5-carboxylic acid (2-imidazol-1-yl-ethyl)-amide |
| 328 | 1-Methyl-2-(6-trifluoromethoxy-benzothiazol-2-ylamino)-1H- |
| | benzoimidazole-5-carboxylic acid (2-pyrazol-1-yl-ethyl)-amide |
| 329 | 1-Methyl-2-(6-trifluoromethoxy-benzothiazol-2-ylamino)-1H- |
| | benzoimidazole-5-carboxylic acid [2-(2-oxo-pyrrolidin-1-yl)- |
| | ethyl]-amide |
| 330 | 1-Methyl-2-(6-trifluoromethoxy-benzothiazol-2-ylamino)-1H- |
| | benzoimidazole-5-carboxylic acid [2-(2-oxo-imidazolidin-1-yl)- |
| | ethyl]-amide |
| 331 | 2-(6-Chloro-benzothiazol-2-ylamino)-1-methyl-1H- |
| | benzoimidazole-5-carboxylic acid [2-(3-oxo-piperazin-1-yl)- |
| | ethyl]-amide |
| 332 | 2-(6-Chloro-benzothiazol-2-ylamino)-1-(2-methoxy-ethyl)-1H- |
| | benzoimidazole-5-carboxylic acid [2-(3-oxo-piperazin-1-yl)- |
| | ethyl]-amide |
| 333 | 1-Methyl-2-(6-trifluoromethoxy-benzothiazol-2-ylamino)-1H- |
| | benzoimidazole-5-carboxylic acid (2-piperidin-1-yl-ethyl)-amide |
| 334 | 2-(6-Chloro-benzothiazol-2-ylamino)-1-methyl-1H- |
| | benzoimidazole-5-carboxylic acid (2-piperidin-1-yl-ethyl)-amide |
| 335 | 2-(6-Chloro-benzothiazol-2-ylamino)-1-methyl-1H- |
| | benzoimidazole-5-carboxylic acid [2-(4,4-difluoro-piperidin-1- |
| | yl)-ethyl]-amide |
| 336 | 2-(6-Chloro-benzothiazol-2-ylamino)-1-methyl-1H- |
| | benzoimidazole-5-carboxylic acid [2-(3,3-difluoro-piperidin- |
| | 1-yl)-ethyl]-amide |
| 337 | 1-Methyl-2-(6-trifluoromethoxy-benzothiazol-2-ylamino)-1H- |
| | benzoimidazole-5-carboxylic acid [2-(4-methyl-piperazin-1-yl)- |
| | ethyl]-amide |
| 338 | 2-(6-Chloro-benzothiazol-2-ylamino)-1-methyl-1H- |
| | benzoimidazole-5-carboxylic acid [2-(4-methyl-piperazin-1-yl)- |
| | ethyl]-amide |
| 339 | 2-(6-Chloro-benzothiazol-2-ylamino)-1-methyl-1H- |
| | benzoimidazole-5-carboxylic acid (2-piperazin-1-yl-ethyl)- |
| | amide hydrochloride |
| 340 | 2-(6-Chloro-benzothiazol-2-ylamino)-1-methyl-1H- |
| | benzoimidazole-5-carboxylic acid (2-morpholin-4-yl-ethyl)-amide |
| 341 | 1-Methyl-2-(6-trifluoromethyl-benzothiazol-2-ylamino)-1H- |
| | benzoimidazole-5-carboxylic acid (2-morpholin-4-yl-ethyl)-amide |
| 342 | 1-Methyl-2-(6-trifluoromethoxy-benzothiazol-2-ylamino)-1H- |
| | benzoimidazole-5-carboxylic acid [2-(1,1-dioxo-thiomorpholin- |
| | 4-yl)-ethyl]-amide |
| 343 | 2-(6-Chloro-benzothiazol-2-ylamino)-1-methyl-1H- |
| | benzoimidazole-5-carboxylic acid (2-amino-ethyl)-amide |
| | hydrochloride |
| 344 | 1-Methyl-2-(6-trifluoromethoxy-benzothiazol-2-ylamino)-1H- |
| | benzoimidazole-5-carboxylic acid (3-amino-propyl)-amide |
| | hydrochloride |
| 345 | 2-(6-Chloro-benzothiazol-2-ylamino)-1-methyl-1H- |
| | benzoimidazole-5-carboxylic acid (3-amino-propyl)-amide |
| | hydrochloride |
| 346 | 1-Methyl-2-(6-trifluoromethoxy-benzothiazol-2-ylamino)-1H- |
| | benzoimidazole-5-carboxylic acid (4-amino-butyl)-amide |
| | hydrochloride |
| 347 | 2-(6-Chloro-benzothiazol-2-ylamino)-1-methyl-1H- |
| | benzoimidazole-5-carboxylic acid (4-amino-butyl)-amide |
| | hydrochloride |
| 348 | 1-Methyl-2-(6-trifluoromethoxy-benzothiazol-2-ylamino)-1H- |
| | benzoimidazole-5-carboxylic acid (3-dimethylamino-propyl)- |
| | amide |
| 349 | 2-(6-Chloro-benzothiazol-2-ylamino)-1-methyl-1H- |
| | benzoimidazole-5-carboxylic acid (3-dimethylamino-propyl)- |
| | amide |
| 350 | 1-Methyl-2-(6-trifluoromethyl-benzothiazol-2-ylamino)-1H- |
| | benzoimidazole-5-carboxylic acid (3-dimethylamino-propyl)- |
| | amide |
| 351 | 1-Methyl-2-(6-trifluoromethoxy-benzothiazol-2-ylamino)-1H- |
| | benzoimidazole-5-carboxylic acid (3-diethylamino-propyl)-amide |
| 352 | 2-(6-Chloro-benzothiazol-2-ylamino)-1-methyl-1H- |
| | benzoimidazole-5-carboxylic acid (3-diethylamino-propyl)-amide |
| 353 | 1-Methyl-2-(6-trifluoromethyl-benzothiazol-2-ylamino)-1H- |
| | benzoimidazole-5-carboxylic acid (3-diethylamino-propyl)-amide |
| 354 | 2-(6-Chloro-benzothiazol-2-ylamino)-1-methyl-1H- |
| | benzoimidazole-5-carboxylic acid (3-pyrrolidin-1-yl-propyl)- |
| | amide |
| 355 | 1-Methyl-2-(6-trifluoromethoxy-benzothiazol-2-ylamino)-1H- |
| | benzoimidazole-5-carboxylic acid [3-(4-methyl-piperazin-1-yl)- |
| | propyl]-amide |
| 356 | 2-(6-Chloro-benzothiazol-2-ylamino)-1-methyl-1H- |
| | benzoimidazole-5-carboxylic acid [3-(4-methyl-piperazin-1-yl)- |
| | propyl]-amide |
| 357 | 1-Methyl-2-(6-trifluoromethyl-benzothiazol-2-ylamino)-1H- |
| | benzoimidazole-5-carboxylic acid [3-(4-methyl-piperazin-1-yl)- |
| | propyl]-amide |
| 358 | 1-Methyl-2-(6-trifluoromethoxy-benzothiazol-2-ylamino)-1H- |
| | benzoimidazole-5-carboxylic acid (3-morpholin-4-yl-propyl)- |
| | amide |
| 359 | 2-(6-Chloro-benzothiazol-2-ylamino)-1-methyl-1H- |
| | benzoimidazole-5-carboxylic acid (3-morpholin-4-yl-propyl)- |
| | amide |
| 360 | 1-Methyl-2-(6-trifluoromethoxy-benzothiazol-2-ylamino)-1H- |
| | benzoimidazole-5-carboxylic acid (4-diethylamino-butyl)-amide |
| 361 | 6-Diethylamino-1-methyl-2-(6-trifluoromethoxy-benzothiazol-2- |
| | ylamino)-1H-benzoimidazole-5-carboxylic acid |
| | dimethylcarbamoylmethyl-amide |
| 362 | 6-Diethylamino-1-methyl-2-(6-trifluoromethoxy-benzothiazol-2- |
| | ylamino)-1H-benzoimidazole-5-carboxylic acid (2-morpholin-4- |
| | yl-ethyl)-amide |
| 363 | 1-Methyl-2-(6-methyl-benzothiazol-2-ylamino)-1H- |
| | benzoimidazole-5-carboxylic acid dimethylcarbamoylmethyl- |
| | amide |
| 364 | 2-(6-Ethoxy-benzothiazol-2-ylamino)-1-methyl-1H- |
| | benzoimidazole-5-carboxylic acid dimethylcarbamoylmethyl- |
| | amide |
| 365 | 2-(6-Isopropyl-benzothiazol-2-ylamino)-1-methyl-1H- |
| | benzoimidazole-5-carboxylic acid dimethylcarbamoylmethyl- |
| | amide |
| 366 | 2-(6-Chloro-benzothiazol-2-ylamino)-1-(2-methoxy-ethyl)-1H- |
| | benzoimidazole-5-carboxylic acid dimethylcarbamoylmethyl- |
| | amide |
| 367 | 2-(6-Chloro-benzothiazol-2-ylamino)-1-(2-methylamino-ethyl)- |
| | 1H-benzoimidazole-5-carboxylic acid dimethylcarbamoylmethyl- |
| | amide hydrochloride |
| 368 | 1-Methyl-2-(6-trifluoromethoxy-benzothiazol-2-ylamino)-1H- |
| | benzoimidazole-5-carboxylic acid ((S)-1-dimethylcarbamoyl-2- |
| | hydroxy-ethyl)-amide |
| 369 | 1-Methyl-2-(6-trifluoromethyl-benzothiazol-2-ylamino)-1H- |
| | benzoimidazole-5-carboxylic acid ((S)-5-amino-1- |
| | dimethylcarbamoyl-pentyl)-amide hydrochloride |
| 370 | 1-Methyl-2-(6-trifluoromethyl-benzothiazol-2-ylamino)-1H- |
| | benzoimidazole-5-carboxylic acid ((S)-5-dimethylamino-1- |
| | dimethylcarbamoyl-pentyl)-amide |
| 371 | 2-(6-Chloro-benzothiazol-2-ylamino)-1-methyl-1H- |
| | benzoimidazole-5-carboxylic acid (2-dimethylcarbamoyl-ethyl)- |
| | amide |
| 372 | 1-Methyl-2-(6-trifluoromethyl-benzothiazol-2-ylamino)-1H- |
| | benzoimidazole-5-carboxylic acid (2-dimethylcarbamoyl- |
| | ethyl)-amide |
| 373 | 2-(6-Chloro-benzothiazol-2-ylamino)-1-methyl-1H- |
| | benzoimidazole-5-carboxylic acid (3-morpholin-4-yl-3-oxo- |
| | propyl)-amide |
| 374 | 1-Methyl-2-(6-trifluoromethyl-benzothiazol-2-ylamino)-1H- |
| | benzoimidazole-5-carboxylic acid (3-morpholin-4-yl-3-oxo- |
| | propyl)-amide |
| 375 | 1-Methyl-2-(6-trifluoromethyl-benzothiazol-2-ylamino)-1H- |
| | benzoimidazole-5-carboxylic acid [3-(4-methyl-piperazin-1-yl)- |
| | 3-oxo-propyl]-amide |
| 376 | 2-(6-Chloro-benzothiazol-2-ylamino)-1-methyl-1H- |
| | benzoimidazole-5-carboxylic acid (3-dimethylcarbamoyl- |
| | propyl)-amide |
| 377 | 2-(6-Chloro-benzothiazol-2-ylamino)-1-methyl-1H- |
| | benzoimidazole-5-carboxylic acid [4-(4-methyl-piperazin-1-yl)- |
| | 4-oxo-butyl]-amide |
| 378 | 4-{[2-(6-Chloro-benzothiazol-2-ylamino)-1-methyl-1H- |
| | benzoimidazole-5-carbonyl]-amino}-trans-cyclohexanecarboxylic |
| | acid |
| 379 | 2-(6-Chloro-benzothiazol-2-ylamino)-1-methyl-1H- |
| | benzoimidazole-5-carboxylic acid (4-trans-dimethylcarbamoyl- |
| | cyclohexyl)-amide |
| 380 | 2-(6-Chloro-benzothiazol-2-ylamino)-1-methyl-1H- |
| | benzoimidazole-5-carboxylic acid methylcarbamoylmethyl-amide |
| 381 | 1-Methyl-2-(6-trifluoromethyl-benzothiazol-2-ylamino)-1H- |
| | benzoimidazole-5-carboxylic acid methylcarbamoylmethyl-amide |
| 382 | 2-(6-Chloro-benzothiazol-2-ylamino)-1-methyl-1H- |
| | benzoimidazole-5-carboxylic acid [((R)-2-hydroxy- |
| | propylcarbamoyl)-methyl]-amide |
| 383 | 2-(6-Chloro-benzothiazol-2-ylamino)-1-methyl-1H- |
| | benzoimidazole-5-carboxylic acid [(2-methanesulfonyl-ethyl |
| | carbamoyl)-methyl]-amide |
| 384 | 2-(6-Chloro-benzothiazol-2-ylamino)-1-methyl-1H- |
| | benzoimidazole-5-carboxylic acid [(tetrahydro-furan-3- |
| | ylcarbamoyl)-methyl]-amide |
| 385 | 2-(6-Chloro-benzothiazol-2-ylamino)-1-methyl-1H- |
| | benzoimidazole-5-carboxylic acid [(tetrahydro-pyran-4- |
| | ylcarbamoyl)-methyl]-amide |
| 386 | 2-(6-Chloro-benzothiazol-2-ylamino)-1-methyl-1H- |
| | benzoimidazole-5-carboxylic acid [(1-methyl-piperidin-4- |
| | ylcarbamoyl)-methyl]-amide |
| 387 | 2-(6-Chloro-benzothiazol-2-ylamino)-1-methyl-1H- |
| | benzoimidazole-5-carboxylic acid ((R)-piperidin-3- |
| | ylcarbamoylmethyl)-amide hydrochloride |
| 388 | 2-(6-Chloro-benzothiazol-2-ylamino)-1-methyl-1H- |
| | benzoimidazole-5-carboxylic acid [((R)-1-methyl-piperidin-3- |
| | ylcarbamoyl)-methyl]-amide |
| 389 | 1-Methyl-2-(6-trifluoromethoxy-benzothiazol-2-ylamino)-1H- |
| | benzoimidazole-5-carboxylic acid [(4-hydroxy-benzylcarbamoyl)- |
| | methyl]-amide |
| 390 | 1-Methyl-2-(6-trifluoromethoxy-benzothiazol-2-ylamino)-1H- |
| | benzoimidazole-5-carboxylic acid {[(2-hydroxy-ethyl)-methyl- |
| | carbamoyl]-methyl}-amide |
| 391 | 2-(6-Chloro-benzothiazol-2-ylamino)-1-methyl-1H- |
| | benzoimidazole-5-carboxylic acid {[(2-hydroxy-ethyl)-methyl- |
| | carbamoyl]-methyl}-amide |
| 392 | 1-Methyl-2-(6-trifluoromethoxy-benzothiazol-2-ylamino)-1H- |
| | benzoimidazole-5-carboxylic acid {[bis-(2-hydroxy-ethyl)- |
| | carbamoyl]-methyl}-amide |
| 393 | 2-(6-Chloro-benzothiazol-2-ylamino)-1-methyl-1H- |
| | benzoimidazole-5-carboxylic acid {[methyl-(tetrahydro-pyran-4- |
| | yl)-carbamoyl]-methyl}-amide |
| 394 | 2-(6-Chloro-benzothiazol-2-ylamino)-1-methyl-1H- |
| | benzoimidazole-5-carboxylic acid [(methyl-pyrrolidin-3-yl- |
| | carbamoyl)-methyl]-amide hydrochloride |
| 395 | 2-(6-Chloro-benzothiazol-2-ylamino)-1-methyl-1H- |
| | benzoimidazole-5-carboxylic acid {[methyl-(1-methyl-pyrrolidin- |
| | 3-yl)-carbamoyl]-methyl}-amide |
| 396 | 2-(6-Chloro-benzothiazol-2-ylamino)-1-methyl-1H- |
| | benzoimidazole-5-carboxylic acid [(methyl-piperidin-3-yl- |
| | carbamoyl)-methyl]-amide hydrochloride |
| 397 | 2-(6-Chloro-benzothiazol-2-ylamino)-1-methyl-1H- |
| | benzoimidazole-5-carboxylic acid (2-oxo-2-pyrrolidin-1-yl- |
| | ethyl)-amide |
| 398 | 1-Methyl-2-(6-trifluoromethoxy-benzothiazol-2-ylamino)-1H- |
| | benzoimidazole-5-carboxylic acid [2-(3-hydroxy-pyrrolidin-1-yl)- |
| | 2-oxo-ethyl]-amide |
| 399 | 2-(6-Chloro-benzothiazol-2-ylamino)-1-methyl-1H- |
| | benzoimidazole-5-carboxylic acid [2-(3-hydroxy-pyrrolidin-1- |
| | yl)-2-oxo-ethyl]-amide |
| 400 | 2-(6-Chloro-benzothiazol-2-ylamino)-1-methyl-1H- |
| | benzoimidazole-5-carboxylic acid [2-((R)-3-hydroxy-pyrrolidin- |
| | 1-yl)-2-oxo-ethyl]-amide |
| 401 | 2-(6-Chloro-benzothiazol-2-ylamino)-1-methyl-1H- |
| | benzoimidazole-5-carboxylic acid [2-((S)-3-hydroxy-pyrrolidin-1- |
| 402 | 1-Methyl-2-(6-trifluoromethyl-benzothiazol-2-ylamino)-1H- |
| | benzoimidazole-5-carboxylic acid [2-((R)-3-hydroxy-pyrrolidin- |
| | 1-yl)-2-oxo-ethyl]-amide |
| 403 | 1-Methyl-2-(6-trifluoromethyl-benzothiazol-2-ylamino)-1H- |
| | benzoimidazole-5-carboxylic acid [2-((S)-3-hydroxy-pyrrolidin-1- |
| | yl)-2-oxo-ethyl]-amide |
| 404 | 2-(6-Chloro-benzothiazol-2-ylamino)-1-(2-methoxy-ethyl)- |
| | 1H-benzoimidazole-5-carboxylic acid [2-((R)-3-hydroxy- |
| | pyrrolidin-1-yl)-2-oxo-ethyl]-amide |
| 405 | 2-(6-Chloro-benzothiazol-2-ylamino)-1-(2-methoxy-ethyl)-1H- |
| | benzoimidazole-5-carboxylic acid [2-((S)-3-hydroxy-pyrrolidin- |
| | 1-yl)-2-oxo-ethyl]-amide |
| 406 | 1-Methyl-2-(6-trifluoromethoxy-benzothiazol-2-ylamino)-1H- |
| | benzoimidazole-5-carboxylic acid [2-((S)-2-hydroxymethyl- |
| | pyrrolidin-1-yl)-2-oxo-ethyl]-amide |
| 407 | 1-Methyl-2-(6-trifluoromethoxy-benzothiazol-2-ylamino)-1H- |
| | benzoimidazole-5-carboxylic acid [2-((3S,4S)-3,4-dihydroxy- |
| | pyrrolidin-1-yl)-2-oxo-ethyl]-amide |
| 408 | 2-(6-Chloro-benzothiazol-2-ylamino)-1-methyl-1H- |
| | benzoimidazole-5-carboxylic acid [2-((R)-3-methoxy- |
| | pyrrolidin-1-yl)-2-oxo-ethyl]-amide |
| 409 | 2-(6-Chloro-benzothiazol-2-ylamino)-1-methyl-1H- |
| | benzoimidazole-5-carboxylic acid [2-((S)-3-methoxy-pyrrolidin- |
| | 1-yl)-2-oxo-ethyl]-amide |
| 410 | 1-Methyl-2-(6-trifluoromethyl-benzothiazol-2-ylamino)-1H- |
| | benzoimidazole-5-carboxylic acid [2-((S)-3-methoxy- |
| | pyrrolidin-1-yl)-2-oxo-ethyl]-amide |
| 411 | 2-(6-Chloro-benzothiazol-2-ylamino)-1-methyl-1H- |
| | benzoimidazole-5-carboxylic acid [2-((S)-3-amino-pyrrolidin- |
| | 1-yl)-2-oxo-ethyl]-amide hydrochloride |
| 412 | 1-Methyl-2-(6-trifluoromethoxy-benzothiazol-2-ylamino)-1H- |
| | benzoimidazole-5-carboxylic acid [2-((S)-3-amino-pyrrolidin-1- |
| | yl)-2-oxo-ethyl]-amide hydrochloride |
| 413 | 1-Methyl-2-(6-trifluoromethoxy-benzothiazol-2-ylamino)-1H- |
| | benzoimidazole-5-carboxylic acid [2-((R)-3-methylamino- |
| | pyrrolidin-1-yl)-2-oxo-ethyl]-amide hydrochloride |
| 414 | 1-Methyl-2-(6-trifluoromethoxy-benzothiazol-2-ylamino)-1H- |
| | benzoimidazole-5-carboxylic acid [2-((S)-3-methylamino- |
| | pyrrolidin-1-yl)-2-oxo-ethyl]-amide hydrochloride |
| 415 | 1-Methyl-2-(6-trifluoromethoxy-benzothiazol-2-ylamino)-1H- |
| | benzoimidazole-5-carboxylic acid [2-((R)-3-dimethylamino- |
| | pyrrolidin-1-yl)-2-oxo-ethyl]-amide |
| 416 | 1-Methyl-2-(6-trifluoromethoxy-benzothiazol-2-ylamino)-1H- |
| | benzoimidazole-5-carboxylic acid [2-((S)-3-dimethylamino- |
| | pyrrolidin-1-yl)-2-oxo-ethyl]-amide |
| 417 | 2-(6-Chloro-benzothiazol-2-ylamino)-1-methyl-1H- |
| | benzoimidazole-5-carboxylic acid [2-((R)-3-dimethylamino- |
| | pyrrolidin-1-yl)-2-oxo-ethyl]-amide |
| 418 | 2-(6-Chloro-benzothiazol-2-ylamino)-1-methyl-1H- |
| | benzoimidazole-5-carboxylic acid [2-((S)-3-dimethylamino- |
| | pyrrolidin-1-yl)-2-oxo-ethyl]-amide |
| 419 | 2-(6-Chloro-benzothiazol-2-ylamino)-1-methyl-1H- |
| | benzoimidazole-5-carboxylic acid [2-((S)-2-dimethylcarbamoyl- |
| | pyrrolidin-1-yl)-2-oxo-ethyl]-amide |
| 420 | 3-Methyl-2-(6-trifluoromethoxy-benzothiazol-2-ylamino)-3H- |
| | imidazo[4,5-b]pyridine-6-carboxylic acid (2-morpholin-4-yl- |
| | 2-oxo-ethyl)-amide |
| 421 | 2-(6-Chloro-benzothiazol-2-ylamino)-1-methyl-1H- |
| | benzoimidazole-5-carboxylic acid (2-morpholin-4-yl-2-oxo- |
| | ethyl)-amide |
| 422 | 1-Methyl-2-(6-trifluoromethyl-benzothiazol-2-ylamino)-1H- |
| | benzoimidazole-5-carboxylic acid (2-morpholin-4-yl-2-oxo- |
| | ethyl)-amide |
| 423 | 2-(6-Chloro-benzothiazol-2-ylamino)-1-methyl-1H- |
| | benzoimidazole-5-carboxylic acid (2-oxo-2-thiomorpholin-4-yl- |
| | ethyl)-amide |
| 424 | 2-(6-Chloro-benzothiazol-2-ylamino)-1-methyl-1H- |
| | benzoimidazole-5-carboxylic acid [2-(1,1-dioxo-thiomorpholin- |
| | 4-yl)-2-oxo-ethyl]-amide |
| 425 | 2-(6-Chloro-benzothiazol-2-ylamino)-1-methyl-1H- |
| | benzoimidazole-5-carboxylic acid (2-oxo-2-piperazin-1-yl-ethyl)- |
| | amide hydrochloride |
| 426 | 2-(6-Chloro-benzothiazol-2-ylamino)-1-methyl-1H- |
| | benzoimidazole-5-carboxylic acid [2-(4-methyl-piperazin-1-yl)- |
| | 2-oxo-ethyl]-amide |
| 427 | 1-Methyl-2-(6-trifluoromethyl-benzothiazol-2-ylamino)-1H- |
| | benzoimidazole-5-carboxylic acid [2-(4-methyl-piperazin-1- |
| | yl)-2-oxo-ethyl]-amide |
| 428 | 2-(6-Chloro-benzothiazol-2-ylamino)-1-(2-methoxy-ethyl)-1H- |
| | benzoimidazole-5-carboxylic acid [2-(4-methyl-piperazin-1-yl)-2- |
| | oxo-ethyl]-amide |
| 429 | 2-(6-Chloro-benzothiazol-2-ylamino)-1-methyl-1H- |
| | benzoimidazole-5-carboxylic acid [2-(4-methanesulfonyl- |
| | piperazin-1-yl)-2-oxo-ethyl]-amide |
| 430 | 2-(6-Chloro-benzothiazol-2-ylamino)-1-methyl-1H- |
| | benzoimidazole-5-carboxylic acid [2-(4-dimethyl sulfamoyl- |
| | piperazin-1-yl)-2-oxo-ethyl]-amide |
| 431 | 1-Methyl-2-(6-trifluoromethoxy-benzothiazol-2-ylamino)-1H- |
| | benzoimidazole-5-carboxylic acid [2-(3-hydroxy-piperidin-1-yl)- |
| | 2-oxo-ethyl]-amide |
| 432 | 1-Methyl-2-(6-trifluoromethyl-benzothiazol-2-ylamino)-1H- |
| | benzoimidazole-5-carboxylic acid [2-((R)-3-hydroxy-piperidin-1- |
| | yl)-2-oxo-ethyl]-amide |
| 433 | 2-(6-Chloro-benzothiazol-2-ylamino)-1-methyl-1H- |
| | benzoimidazole-5-carboxylic acid [2-((S)-3-hydroxy-piperidin- |
| | 1-yl)-2-oxo-ethyl]-amide |
| 434 | 1-Methyl-2-(6-trifluoromethyl-benzothiazol-2-ylamino)-1H- |
| | benzoimidazole-5-carboxylic acid [2-((S)-3-hydroxy-piperidin- |
| | 1-yl)-2-oxo-ethyl]-amide |
| 435 | 1-Methyl-2-(6-trifluoromethoxy-benzothiazol-2-ylamino)-1H- |
| | benzoimidazole-5-carboxylic acid [2-(4-hydroxy-piperidin- |
| | 1-yl)-2-oxo-ethyl]-amide |
| 436 | 2-(6-Chloro-benzothiazol-2-ylamino)-1-methyl-1H- |
| | benzoimidazole-5-carboxylic acid [2-(4-hydroxy-piperidin-1- |
| | yl)-2-oxo-ethyl]-amide |
| 437 | 1-Methyl-2-(6-trifluoromethyl-benzothiazol-2-ylamino)-1H- |
| | benzoimidazole-5-carboxylic acid [2-(4-hydroxy-piperidin-1- |
| | yl)-2-oxo-ethyl]-amide |
| 438 | 2-(6-Chloro-benzothiazol-2-ylamino)-1-(2-methoxy-ethyl)-1H- |
| | benzoimidazole-5-carboxylic acid [2-(4-hydroxy-piperidin-1- |
| | yl)-2-oxo-ethyl]-amide |
| 439 | 1-Methyl-2-(6-trifluoromethoxy-benzothiazol-2-ylamino)-1H- |
| | benzoimidazole-5-carboxylic acid [2-(2-hydroxymethyl-piperidin- |
| | 1-yl)-2-oxo-ethyl]-amide |
| 440 | 1-Methyl-2-(6-trifluoromethoxy-benzothiazol-2-ylamino)-1H- |
| | benzoimidazole-5-carboxylic acid [2-(3-hydroxymethyl- |
| | piperidin-1-yl)-2-oxo-ethyl]-amide |
| 441 | 1-Methyl-2-(6-trifluoromethoxy-benzothiazol-2-ylamino)-1H- |
| | benzoimidazole-5-carboxylic acid [2-(4-hydroxymethyl- |
| | piperidin-1-yl)-2-oxo-ethyl]-amide |
| 442 | 2-(6-Chloro-benzothiazol-2-ylamino)-1-methyl-1H- |
| | benzoimidazole-5-carboxylic acid [2-(4-hydroxymethyl- |
| | piperidin-1-yl)-2-oxo-ethyl]-amide |
| 443 | 2-(6-Chloro-benzothiazol-2-ylamino)-1-(2-methoxy-ethyl)-1H- |
| | benzoimidazole-5-carboxylic acid [2-(4-hydroxymethyl- |
| | piperidin-1-yl)-2-oxo-ethyl]-amide |
| 444 | 2-(6-Chloro-benzothiazol-2-ylamino)-1-methyl-1H- |
| | benzoimidazole-5-carboxylic acid [2-((S)-3-methoxy-piperidin-1- |
| | yl)-2-oxo-ethyl]-amide |
| 445 | 1-Methyl-2-(6-trifluoromethyl-benzothiazol-2-ylamino)-1H- |
| | benzoimidazole-5-carboxylic acid [2-((S)-3-methoxy-piperidin-1- |
| | yl)-2-oxo-ethyl]-amide |
| 446 | 1-Methyl-2-(6-trifluoromethyl-benzothiazol-2-ylamino)-1H- |
| | benzoimidazole-5-carboxylic acid [2-((R)-3-methoxy-piperidin-1- |
| | yl)-2-oxo-ethyl]-amide |
| 447 | 1-Methyl-2-(6-trifluoromethoxy-benzothiazol-2-ylamino)-1H- |
| | benzoimidazole-5-carboxylic acid [2-(4-methoxymethyl- |
| | piperidin-1-yl)-2-oxo-ethyl]-amide |
| 448 | 1-Methyl-2-(6-trifluoromethoxy-benzothiazol-2-ylamino)-1H- |
| | benzoimidazole-5-carboxylic acid [2-(4-fluoromethyl-piperidin-1- |
| | yl)-2-oxo-ethyl]-amide |
| 449 | 1-Methyl-2-(6-trifluoromethoxy-benzothiazol-2-ylamino)-1H- |
| | benzoimidazole-5-carboxylic acid [2-oxo-2-(4-trifluoromethyl- |
| | piperidin-1-yl)-ethyl]-amide |
| 450 | 1-Methyl-2-(6-trifluoromethoxy-benzothiazol-2-ylamino)-1H- |
| | benzoimidazole-5-carboxylic acid [2-(4-cyano-piperidin-1- |
| | yl)-2-oxo-ethyl]-amide |
| 451 | 1-Methyl-2-(6-trifluoromethoxy-benzothiazol-2-ylamino)-1H- |
| | benzoimidazole-5-carboxylic acid [2-(4-carbamoyl-piperidin-1- |
| | yl)-2-oxo-ethyl]-amide |
| 452 | 1-Methyl-2-(6-trifluoromethoxy-benzothiazol-2-ylamino)-1H- |
| | benzoimidazole-5-carboxylic acid {2-oxo-2-[4-(pyrimidin-2- |
| | yloxy)-piperidin-1-yl]-ethyl}-amide |
| 453 | 1-Methyl-2-(6-trifluoromethoxy-benzothiazol-2-ylamino)-1H- |
| | benzoimidazole-5-carboxylic acid [2-(4-methylamino-piperidin-1- |
| | yl)-2-oxo-ethyl]-amide hydrochloride |
| 454 | 2-(6-Chloro-benzothiazol-2-ylamino-1-methyl-1H- |
| | benzoimidazole-5-carboxylic acid [2-(4-methylamino-piperidin-1- |
| | yl)-2-oxo-ethyl]-amide hydrochloride |
| 455 | 1-Methyl-2-(6-trifluoromethoxy-benzothiazol-2-ylamino)-1H- |
| | benzoimidazole-5-carboxylic acid [2-(4-dimethylamino- |
| | piperidin-1-yl)-2-oxo-ethyl]-amide |
| 456 | 2-(6-Chloro-benzothiazol-2-ylamino)-1-methyl-1H- |
| | benzoimidazole-5-carboxylic acid [2-(4-dimethylamino- |
| | piperidin-1-yl)-2-oxo-ethyl]-amide |
| 457 | 2-(6-Chloro-benzothiazol-2-ylamino)-1-methyl-1H- |
| | benzoimidazole-5-carboxylic acid [2-(4-methylaminomethyl- |
| | piperidin-1-yl)-2-oxo-ethyl]-amide hydrochloride |
| 458 | 1-Methyl-2-(6-trifluoromethoxy-benzothiazol-2-ylamino)-1H- |
| | benzoimidazole-5-carboxylic acid [2-(4-dimethylaminomethyl- |
| | piperidin-1-yl)-2-oxo-ethyl]-amide |
| 459 | 2-(6-Chloro-benzothiazol-2-ylamino)-1-methyl-1H- |
| | benzoimidazole-5-carboxylic acid [2-(4-dimethylaminomethyl- |
| | piperidin-1-yl)-2-oxo-ethyl]-amide |
| 460 | 2-(6-Chloro-benzothiazol-2-ylamino)-1-methyl-1H- |
| | benzoimidazole-5-carboxylic acid [2-(3-methylaminomethyl- |
| | piperidin-1-yl)-2-oxo-ethyl]-amid hydrochloride |
| 461 | 2-(6-Chloro-benzothiazol-2-ylamino)-1-methyl-1H- |
| | benzoimidazole-5-carboxylic acid [2-(3-dimethylaminomethyl- |
| | piperidin-1-yl)-2-oxo-ethyl]-amide |
| 462 | 2-(6-Chloro-benzothiazol-2-ylamino)-1-methyl-1H- |
| | benzoimidazole-5-carboxylic acid ((S)-1-dimethylcarbamoyl- |
| | ethyl)-amide |
| 463 | 1-Methyl-2-(6-trifluoromethyl-benzothiazol-2-ylamino)-1H- |
| | benzoimidazole-5-carboxylic acid ((S)-1-dimethylcarbamoyl- |
| | ethyl)-amide |
| 464 | 2-(6-Chloro-benzothiazol-2-ylamino)-1-methyl-1H- |
| | benzoimidazole-5-carboxylic acid [(S)-1-methyl-2-(4-methyl- |
| | piperazin-1-yl)-2-oxo-ethyl]-amide |
| 465 | 2-(6-Chloro-benzothiazol-2-ylamino)-1-methyl-1H- |
| | benzoimidazole-5-carboxylic acid ((S)-1-methyl-2-morpholin-4- |
| | yl-2-oxo-ethyl)-amide |
| 466 | 1-Methyl-2-(6-trifluoromethyl-benzothiazol-2-ylamino)-1H- |
| | benzoimidazole-5-carboxylic acid ((S)-1-methyl-2-morpholin-4- |
| | yl-2-oxo-ethyl)-amide |
| 467 | 2-(6-Chloro-benzothiazol-2-ylamino)-1-methyl-1H- |
| | benzoimidazole-5-carboxylic acid ((R)-1-dimethylcarbamoyl- |
| | ethyl)-amide |
| 468 | 1-Methyl-2-(6-trifluoromethyl-benzothiazol-2-ylamino)-1H- |
| | benzoimidazole-5-carboxylic acid ((R)-1-dimethylcarbamoyl- |
| | ethyl)-amide |
| 469 | 2-(6-Chloro-benzothiazol-2-ylamino)-1-methyl-1H- |
| | benzoimidazole-5-carboxylic acid [(R)-1-methyl-2-(4-methyl- |
| | piperazin-1-yl)-2-oxo-ethyl]-amide |
| 470 | 1-Methyl-2-(6-trifluoromethyl-benzothiazol-2-ylamino)-1H- |
| | benzoimidazole-5-carboxylic acid [(R)-1-methyl-2-(4-methyl- |
| | piperazin-1-yl)-2-oxo-ethyl]-amide |
| 471 | 2-(6-Chloro-benzothiazol-2-ylamino)-1-methyl-1H- |
| | benzoimidazole-5-carboxylic acid ((R)-1-methyl-2-morpholin-4- |
| | yl-2-oxo-ethyl)-amide |
| 472 | 1-Methyl-2-(6-trifluoromethyl-benzothiazol-2-ylamino)-1H- |
| | benzoimidazole-5-carboxylic acid ((R)-1-methyl-2-morpholin-4- |
| | yl-2-oxo-ethyl)-amide |

Compounds in Table A and within the genus of Formula (I) may be prepared as described in WO '018 or other methods apparent to one of skill in the art.

In another embodiment, the present invention provides a compound of Formula (I) or a pharmaceutically acceptable salt thereof for use in medicine. In another embodiment, the invention provides a compound or a pharmaceutically acceptable salt of any one of embodiments 1 to 250 for use in medicine. In another embodiment, the present invention provides a compound of Formula (I) or a pharmaceutically acceptable salt thereof for use in preparation of a medicament to treat one or more of the conditions described herein. In another embodiment, the invention provides a compound or a pharmaceutically acceptable salt of any one of embodiments 1 to 250 for use in preparation of a medicament to treat one or more of the conditions described herein.

B. Co-Administration

The pharmaceutical compositions disclosed herein may be co-formulated with one or more therapeutic agents or may be formulated for co-administration with a separate formulation of a therapeutic agent. An appropriate time course for sequential administration may be chosen by the physician, according to such factors as the nature of a patient's illness, and the patient's condition. In certain embodiments, sequential administration includes the co-administration of one or more additional therapeutics agents within a period of one week, 72 hours, 48 hours, 24 hours, or 12 hours. The additional therapeutic agent may include those described in Section II.B above.

C. Effective Amounts

In some embodiments, the compositions are administered in an amount effective to induce a pharmacological, physiological, or molecular effect compared to a control that is not administered the composition. In some embodiments, a pharmaceutical composition comprising a compound of Formula (I) or a pharmaceutically acceptable salt thereof is administered to a subject in need thereof in an amount to reduce osteoclastogenesis in the subject or to reduce osteoclast function in the subject. For example, a decrease in osteoclast function may be measured by a reduction in expression of one or more of osteoclast marker genes such as, but not limited to, Atp6v0d2, Matrix metalloprotein 9, Oscar, Cathepsin K, Dcstamp, and Trap. In some embodiments, a pharmaceutical composition comprises a compound of Formula (I) or a pharmaceutically acceptable salt thereof is administered to a subject in need thereof in an amount to decrease the loss of bone density, maintain bone density, or increase bone density.

Suitable controls are known in the art and can be determined based on the disease to be treated. Suitable controls include, but are not limited to a subject, or similarly situated subjects without a related disorder; or a condition or status of a subject with the disease or disorder prior to initiation of the treatment.

In some additional embodiments, a pharmaceutical composition comprising a compound of Formula (I) or a pharmaceutically acceptable salt thereof is administered to a subject in need thereof in an effective amount to improve one or more pharmacological, physiological, or molecular effects, or to reduce or alleviate one or more symptoms of the disease or disorder compared to a subject treated with a different therapeutic agent.

D. Dosages and Dosage Regimes

An appropriate dose of a compound of Formula (I) or a pharmaceutically acceptable salt thereof for use in pharmaceutical formulations of the present invention may be between 0.1 mg and 15 mg per kg. In another embodiment, where the subject is a human the dose may be between 1 mg and 1000 mg. In another embodiment, a compound of Formula (I) or a pharmaceutically acceptable salt thereof is administered at a daily or weekly dose that is less than or equal to 100, 95, 90, 85, 80, 75, 70, 65, 60, 55, 50, 45, 40, 35, 30, 25, 20, 15, 10, or 5 mg.

E. Formulations

The pharmaceutical compositions comprising a compound of Formula (I) or a pharmaceutically acceptable salt thereof may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In another embodiment, formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

In another embodiment, the composition may comprise an aqueous suspension. Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Also, oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

In another embodiment, the pharmaceutical compositions of the present invention may comprise a syrup or elixir. Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The pharmaceutical compositions of the present invention may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

In an embodiment, for topical use, creams, ointments, jellies, solutions of suspensions, etc., containing the compounds of the invention may be employed. For the purpose of this application, topical applications shall include mouth washes and gargles.

Pharmaceutically-acceptable salts of compounds of Formula (I), where a basic or acidic group is present in the structure, are also included within the scope of the invention. The term “pharmaceutically acceptable salts” refers to salts of the compounds of this invention which are not biologically or otherwise undesirable and are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isethionate, Lactate, Lactobionate, Laurate, Malate, Maleate, Mandelate, Mesylate, Methylbromide, Methylnitrate, Methyl sulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts recited in Stephen M. Berge, et al., J, Pharm, Sci. Vol 66(1), pp. 1-19(1977).

Thus, in another embodiment, the invention provides a pharmaceutical composition comprising a compound of Formula (I) or a pharmaceutically acceptable salt thereof, and a pharmaceutically acceptable carrier. In another embodiment, the invention provides a pharmaceutical composition comprising a compound or pharmaceutically acceptable salt of any one of embodiments 1 to 250 and a pharmaceutical acceptable carrier.

### IV. Methods of Diagnosis

The methods of treatment disclosed herein can include a first step of selecting a subject for treatment. In some embodiments, the subject is selected for treatment when the subject exhibits one or more of the clinical symptoms of bone loss or bone disease. In some embodiments, the subject is selected for treatment when the subject exhibits a genetic or biochemical indicator or has a risk factor for bone loss or bone disease.

In some embodiments, the subject is selected when a combination of clinical symptoms and genetic or biochemical alterations or risk factors are identified. In some embodiments, the subject is selected based on one or more clinical symptoms, or one or more genetic or biochemical alterations or risk factors. For example, subjects can be selected for treatment based on the identification of a certain amount of bone loss, a certain age, sex, upregulation of or certain levels of one or more biomarkers or genes associated with osteoclastogenesis or osteoclast activity.

The diagnosis of and monitoring progress of bone related disease, such as osteoporosis or other diseases described herein, may require information about bone turnover and bone mass. Determinations of bone turnover have been performed using standard serum, urine and/or sweat laboratory tests including fasting calcium/creatinine, hydroxyproline, alkaline phosphatase and/or osteocalcin/bone growth protein utilizing standard high pressure liquid chromatography (HPLC) techniques. For example, whenever bone formation occurs (calcium deposition) or bone resorption occurs (calcium breakdown), various chemical reactions occur within the body that elevate the presence of certain indicators in the blood and urine suggesting changes in the calcium/bone mineral status. Biomarkers may lack information on the severity or stage of a disease and on the morphological condition of an organ or tissue.

Several bone specific assays have been developed which enable bone turnover to be evaluated with an ELISA/EMIT immunoassay format. Descriptions of these immunoassay formats can be found in U.S. Pat. Nos. 5,320,970, 5,300,434, and 5,140,103. The labeling for the new assays use a biochemical marker to quantify bone resorption and/or formation and provides information on bone turnover.

For diagnosis of bone diseases, collagen breakdown products may be detected in serum or urine by using two or more immunoassays, and forming a ratio between the concentration of one fragment and a second fragment to form an index to determine the rate of bone resorption. In another method of forming an index of biomarker results, a ratio of free lysyl pyridinoline cross-links and creatinine content to form a urinary index of bone resorption are used to diagnose bone disease.

Bone mass determinations, on the other hand, have been traditionally performed by using various x-ray based techniques including single and dual-photon absorptiometry (SPA and DP A), quantitative computed tomography (QCT), and dual-energy absorptiometry (DXA). Imaging tests such as x-rays, ultrasound, computed tomography and MRI can provide detailed information about the morphological condition of an organ or a tissue and on the severity or the stage of a disease process. However, such imaging techniques typically lack information on the metabolic activity of various tissues and organs and, in diseased states, cannot give an estimate of the rate of progression or the prognosis of a disease.

A. Bone mineral density test A clinical practice for diagnosing bone disease or bone mass involves a skeletal survey, which comprises a series of plain film x-rays or other x-ray techniques including single and dual-photon absorptiometry (SPA and DP A). Other imaging modalities have been studied including CT, dual-energy absorptiometry (DXA), MRI, and PET. Imaging tests such as x-rays, ultrasound, computed tomography and MRI can provide detailed information about the morphological condition of an organ or a tissue and on the severity or the stage of a disease process. An example of methods for analyzing x-ray images to evaluate bone condition may be found in U.S. Pat. No. 8,639,009.

T-score is a subject's bone density compared with what is normally expected in a healthy young adult of someone's sex. T-score is the number of standard deviations that bone density is above or below the average; a T-score of −1 and above—a subject's bone density is considered normal; a T-score between −1 and −2.5 is a sign of osteopenia, a condition in which bone density is below normal and may lead to osteoporosis; a T-score of −2.5 and below indicates likely osteoporosis in a subject.

Z-score is the number of standard deviations above or below what is normally expected for someone of a person's age, sex, weight, and ethnic or racial origin. If Z-score is significantly higher or lower than the average, it may suggest that something other than aging is causing abnormal bone loss.

B. Bodily Fluid Tests

Bodily fluid-based diagnostic tests may also be used. Dysregulated bone metabolism can be detected in a patient's blood or urine by measuring biochemical resorptive markers such as, for example, N-terminal cross-linked telopeptide (NTx), C-terminal cross-linked telopeptide (CTx), soluble RANKL, and the ratio of soluble RANKL to osteoprotegrin (sRANKL/OPG). Bodily fluids may also be used to measure the amount of bone microparticles. Hydroxyapatite may be used as a biomarker of bone microparticles. In another embodiment, a bodily fluid-based diagnostic text may determine whether a subject under expresses microRNA-140 (miR-140) and/or over expresses let-7 miRNAs.

Additional markers that may be useful for the monitoring or evaluation of a subject include serum total alkaline phosphatase, serum bone-specific alkaline phosphatase, serum osteocalcin, serum C-terminal propeptide of type 1 procollagen C1NP or serum N-terminal propeptide of type I procollagen (P1NP) [to monitor bone formation], urinary hydroxyproline, urinary total pyridinoline (PYD), urinary free deoxypyridinoline (DPD), urinary cross-linked N-terminal telopeptides of type 1 collagen (NTx), urinary or serum cross-linked C-terminal telopeptides of type 1 collagen (CTx), bone sialoprotein (BSP), and tartrate-resistant acid phosphatase 5b (TRACP-5b) [to monitor bone resorption]

Reference values for any test or biomarker may be what is normally expected in a healthy young adult of someone's sex, or may be what is normally expected for someone of a person's age, sex, weight, and ethnic or racial origin. Values greater or less than 10%, 20%, 40%, or 50%, or greater or less than 1, 1.5, 2.0, 2.5 standard deviations from normal values may indicate a subject at risk of or suffering from a bone disease as described herein.

### EXAMPLES

### Materials and Methods

1. Compounds

Recombinant RANKL was purchased from Wako Pure Chemical (Osaka, Japan). The compounds used in the studies were the compounds of Examples 73, 134, 50, and 236 in Table A above, and these compounds were designated as HPP-1, HPP-2, HPP-3 and HPP-4, respectively).

2. Cells

Mouse monocyte cell line RAW264.7 was obtained from the Riken Bioresource Center (Tsukuba, Japan).

3. Cell Culture

RAW264.7 cells were cultured in α-modified Eagle's medium (Wako Pure Chemical) that contained 10% fetal bovine serum (FBS; Atlas Biologicals, Fort Collins, Colo.) and supplemented with antibiotics (100 U/mL of penicillin and 100 μg/mL of streptomycin). All cells were cultured at 37° C. in a 5% CO₂ incubator.

4. Cell Viability Assay

Cytotoxicity of test compounds HPP-1, -2, -3, and -4 was examined using the AlamarBlue™ Cell Viability Reagent (Thermo Fisher Scientific, San Jose, Calif.). In brief, RAW264.7 cells were plated on 24-well plates and were cultured with various concentration of the test compounds for 1 day. Solution was added to the culture and fluorescence intensity (excitation: 530 nm, emission: 590 nm) was measured using the Synergy HTX Multi-Mode plate Reader (BioTek Japan, Tokyo, Japan) after 1 hour.

5. Osteoclastogenesis Assay

Cells were plated on 96-well plates (RAW264.7 cells: 103 cells/well) in triplicate in the presence or absence of recombinant RANKL (100 ng/ml) with or without the test compounds. After 4 days culture, cells were stained for tartrate-resistant acid phosphatase (TRAP) using an acid phosphatase kit (Sigma-Aldrich, St. Louis, Mo.). Dark red multinucleated cells (>3 nuclei) were counted as TRAP-positive multinucleated cells.

6. Real-Time RT-PCR Analysis

RNA was extracted from RAW264.7 cells using the GenElute mammalian total RNA Miniprep kit (Sigma-Aldrich, St. Louis, Mo.) with on-column genomic DNA digestion according to the manufacturer's instructions. For antioxidant gene expression analysis, RNA was extracted at 1 day after test compound treatment, and the gene expressions of HMOX1 and NQO1 was analyzed. For osteoclast marker gene expression analysis, RNA was extracted at 4 days after RANKL stimulation, and the gene expressions of Atp6v0d2, Cathepsin K, Matrix metalloproteinase 9, Trap, Dcstamp, and Oscar were analyzed. After measurement of the RNA concentration, isolated RNA (500 ng each) was reverse transcribed with iScript cDNA-Supermix (Bio-Rad Laboratories, Hercules, Calif.). Real-time RT-PCR was performed with SsoFast EvaGreen-Supermix (Bio-Rad Laboratories). PCR primers used in the experiments were from PrimerBank and were described in H. Kanzaki, et al. “The Keap1/Nrf2 protein axis plays a role in osteoclast differentiation by regulating intracellular reactive oxygen species signaling,” The Journal of Biological Chemistry 288(32) (2013) 23009-20. Fold changes of gene of interest were calculated by using the 8-8 Ct method with ribosomal protein S18 as reference gene. Data shown are representative of 3 independent experiments performed in triplicate.

7. Resorption Assay

RAW264.7 cells were plated on synthesized calcium phosphate substrate (bone resorption assay plate; PG Research, Tokyo, Japan) and stimulated with RANKL in the presence or absence of test compounds. After 7 days of cultivation, cells were removed with bleach, and the calcium phosphate substrate was washed with distilled water and then dried. Photographs were taken, and the average of the resorbed area per field was calculated from 12 images of each sample, using ImageJ software (National Institutes of Health, Bethesda).

8. Preparation of Nuclear and Cytoplasmic Protein Lysate

Nuclear protein lysate was prepared from RAW 264.7 cells using the LysoPure™ Nuclear and Cytoplasmic Extractor Kit (Wako, Osaka, Japan) according to the manufacturer's instructions. Nuclear protein samples were extracted after 6 hrs of Compound HPP-4 treatment. Briefly, cultured cells were washed with PBS and treated with cell lysis buffer. After centrifugation, nuclear pellet was washed twice and lysed with nuclear lysis reagent. After centrifugation, the supernatant was used as the nuclear protein extract. The protein concentrations of each of the nuclear lysates were measured with the Quick Start™ protein assay kit (Bio-Rad), and the concentrations were adjusted to be the same. Cytoplasmic protein samples were extracted after 1 day of Compound HPP-4 treatment using lysis buffer (5 mM EDTA, 10% glycerol, 1% Triton X-100, 0.1% SDS, 1% NP-40 in PBS) containing proteinase inhibitor cocktail (Wako, Osaka, Japan). Protein concentration in each of the cytoplasmic protein lysates was measured with Pierce BCA Protein Assay Kit (Thermo Fisher Scientific, Waltham, Mass.), and the concentrations were adjusted to be the same.

9. Western Blot Analysis

Prepared protein lysates were subjected to electrophoresis on TGX Precast gel (Bio-Rad Laboratories), and the proteins were transferred to a PVDF membrane using a Trans-Blot® Turbo™ (Bio-Rad Laboratories). After washing, the membrane was blocked with PDVF Blocking Reagent® (Toyobo Co. Ltd, Osaka, Japan), and then incubated with the primary antibody in Can Get Signal® Solution-1 (Toyobo Co. Ltd). After thorough washing with PBS containing 0.5% Tween-20 (PBS-T), the membrane was incubated with horse-radish peroxidase-conjugated secondary antibody in Can Get Signal Solution-2 (Toyobo Co. Ltd), and washed with PBS-T. Chemiluminescence was produced using Luminata Forte (EMD Millipore Corporation, Billerica, Mass.) and detected with LumiCube (Liponics, Tokyo, Japan). The primary antibodies for these experiments were anti-Nrf2 (1/1000 dilution; Santa Cruz Biotechnology Inc., Santa Cruz, Calif.), anti-histone H3 (1/4000; Cell Signaling Technology Japan, Tokyo, Japan).

10. Intracellular ROS Detection

Cells were pretreated with or without Compound HPP-4 for 1 h, stimulated with soluble RANKL for 6 h, washed with PBS, and collected. Cells were then incubated with fluorescent superoxide probe (BES-So-AM; Wako Pure Chemical) on ice. After washing, intracellular ROS was detected using an AccuriC6 flow cytometer (BD Biosciences, San Jose, Calif., USA). The viable monocyte/macrophage fraction was gated on a forward scatter/side scatter plot, and ROS levels were monitored in the FL-1 channel. Data shown are representative of 3 independent experiments performed in triplicated.

11. In Vivo Bone Destruction Model

The animal experimental protocol was reviewed and approved by the Institutional Animal Care and Use committee, Tsurumi University (approval number; 28A030). All animals were treated ethically, and animal experiments were performed in compliance with the Regulations for Animal Experiments and Related Activities at Tsurumi University. The calvarial bone destruction mouse model, induced by repeated LPS injections. Twenty 7-week-old male BALB/c mice (Clea Japan, Tokyo, Japan) were used. Mice were randomly assigned to four groups (n=5 each): a Dimethyl sulfoxide (DMSO)-injected group (control group; 10 μl PBS+2 μl DMSO), a HPP-4-injected group (HPP-4 group; 10 μl PBS+2 μl of Cpd. HPP-4 2 μM), an LPS-induced bone resorption group (LPS group; 10 μl of 1 μg/μl LPS+2 μl DMSO) and an LPS-induced bone resorption and Cpd. HPP-4-injected group (LPS+HPP-4 group; 10 μl of 1 μg/μl LPS+2 μl of HPP-4 2 μM), Injections were performed under anaesthesia with a 30-gauge needle at a point on the midline of the skull located between the eyes on days 1, 3, 5, 7, and 9. On day 11, mice were sacrificed by cervical dislocation and cranial tissue samples were fixed overnight with 4% paraformaldehyde in PBS.

12. Micro-Computed Tomography Analysis for Bone Destruction

Fixed cranial tissue samples were subsequently scanned with an X-ray microcomputed tomography (pCT) system (inspeXio SMX-225CT; Shimadzu Corp., Kyoto, Japan). After reconstitution, the DICOM files were rendered into three-dimensional images using Pluto software. Percentage of resorbed area, calculated from the ratio of the number of pixels in the resorbed area in the cranial bone to the number of pixels in the total analyzed image of the cranial bone, was calculated with the ImageJ software. The region of interest was set between the fronto-parietal (coronal) suture and parietooccipital (lambdoidal) suture.

13. Statistical Analysis

All data are presented as the mean±standard deviation from three independent experiments. ANOVA and Tukey's HSD test were used for evaluating the statistical significance (SPSS® 11.0J; IBM, Chicago, Ill.). P<0.05 was considered to be statistically significant.

### Results

1. Assessment of Cytotoxicity of Test Compounds

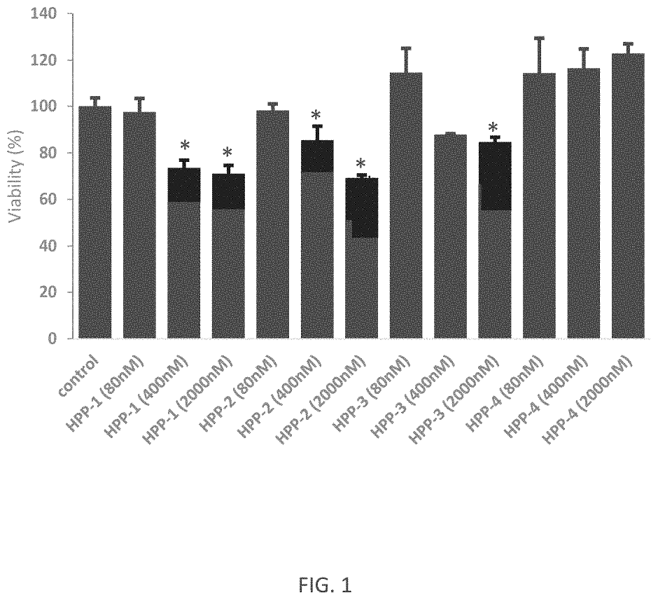

Test compounds HPP-1 to HPP-4 were examined for cytotoxicity against RAW264.7 cells. The results are reported as average percentage of control and are summarized in Table 1 below. There was no statistically significant difference in cell viability at 80 nM for test compounds HPP-1 to HPP-4, but the highest tested concentration of test compounds HPP-1 to HPP-4 (2000 nM) exhibited cytotoxicity compared with the control (FIG. 1).

TABLE 1
Percent of Cell Viability Relative to Control

| | 80 nM of Cpd | 400 nM of Cpd | 2000 nM of Cpd |
|---|---|---|---|
| | (Ave % of control, | (Ave % of control, | (Ave % of control, |
| Cpd. | stdev) | stdev) | stdev) |
| HPP-1 | 97.51 (5.87) | 73.63 (3.32) | 71.14 (3.56) |
| HPP-2 | 98.21 (2.77) | 85.57 (5.97) | 69.35 (1.21) |
| HPP-3 | 114.73 (10.40) | 87.86 (0.34) | 84.78 (2.03) |
| HPP-4 | 114.53 (14.91) | 116.62 (8.25) | 122.89 (4.18) |

Control = 100.00% (stdev 3.59)

2. Test Compounds Suppressed RANKL-Mediated Osteoclastogenesis

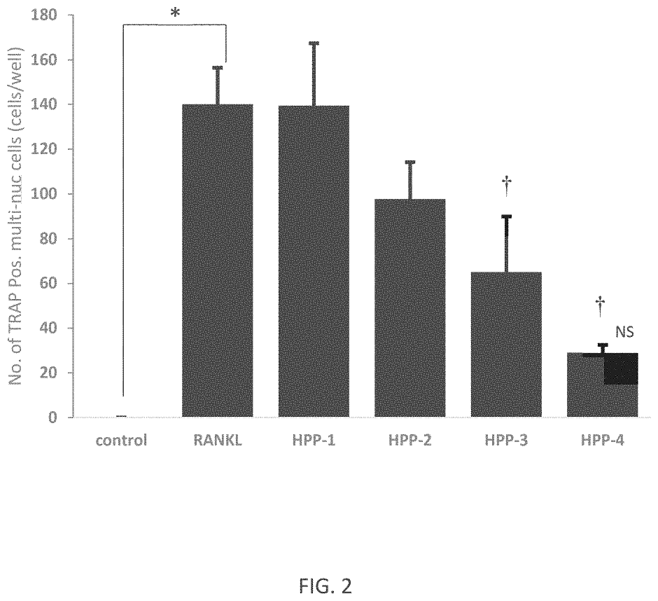

Test compounds HPP-1 to HPP-4 (400 nM) were also examined for any an inhibitory effect on RANKL-mediated osteoclastogenesis. RANKL (100 ng/mL) stimulation of RAW264.7 cells induced a high number of TRAP-positive multinucleated cells, compared with the control, indicating increased osteoclastogenesis. Compounds HPP-1 and HPP-2 had no effect on the number of TPAP-positive multinucleated cells, but compounds HPP-3 and HPP-4 decreased the number of TPAP-positive multinucleated cells. Among them, HPP-4 decreased the number of TPAP-positive multinucleated cells to that of control. These results are summarized in Table 2 below and in FIG. 2. These results indicated that Compound HPP-4 strongly inhibited osteoclast differentiation in RAW264.7 cells in vitro.

TABLE 2
TRAP Staining

| | No. of TRAP-positive | |
|---|---|---|
| | multinucleated cells | |
| Cell Treatment | (Cells/Well) | St. Dev. |
| Control | 0.00 | 0.00 |
| RANKL | 140.00 | 16.52 |
| RANKL + HPP-1 (400 nM) | 139.33 | 28.11 |
| RANKL + HPP-2 (400 nM) | 97.67 | 16.50 |
| RANKL + HPP-3 (400 nM) | 65.00 | 24.98 |
| RANKL + HPP-4 (400 nM) | 29.00 | 3.61 |

3. Test Compounds Suppressed Osteoclast Function

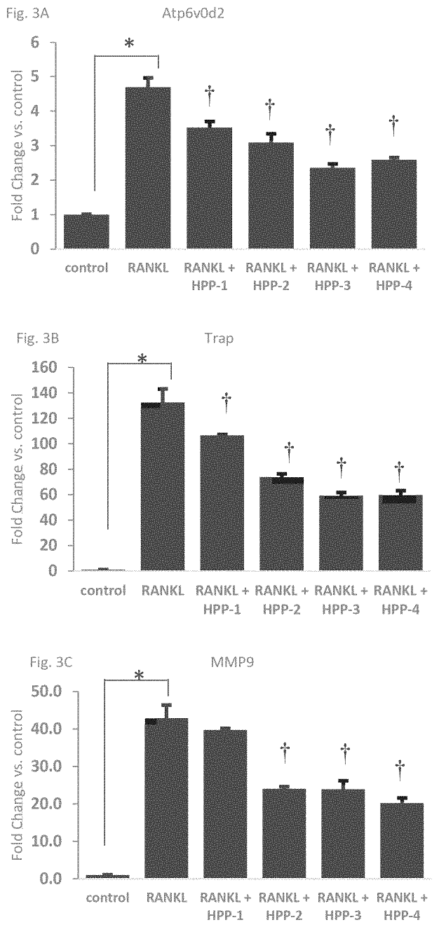

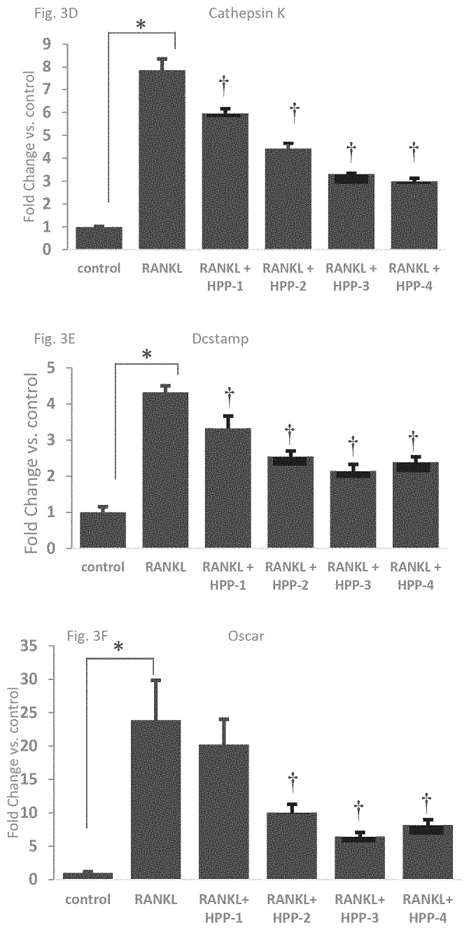

To further examine the inhibitory effect of test compounds HPP-1 to HPP-4 on osteoclastogenesis, the gene expression of several osteoclast differentiation markers in RAW264.7 cells was examined by real-time RT-PCR. RANKL induced the expression of osteoclast marker genes and RANKL-mediated upregulation of these genes was suppressed by Compounds HPP-2, HPP-3 and HPP-4. However, Compound HPP-1 showed weak suppression for these expression, and there were no significant differences in Atp6v0d2, Mmp9, and Oscar. These results are summarized in Tables 3A-3F below and in FIGS. 3A-3F.

### Osteoclast Marker Gene Expression Data (Tables 3A-3F)

TABLE 3A
ATP6vd2

| | Fold Change from | |
|---|---|---|
| Cell Treatment | Control (Ave) | St. Dev. |
| Control | 1.00 | 0.01 |
| RANKL | 4.69 | 0.27 |
| RANKL + HPP-1 (400 nM) | 3.52 | 0.18 |
| RANKL + HPP-2 (400 nM) | 3.09 | 0.26 |
| RANKL + HPP-3 (400 nM) | 2.36 | 0.12 |
| RANKL + HPP-4 (400 nM) | 2.59 | 0.06 |

TABLE 3B
TRAP

| | Fold Change from | |
|---|---|---|
| Cell Treatment | Control (Ave) | St. Dev. |
| Control | 1.00 | 0.15 |
| RANKL | 132.47 | 10.65 |
| RANKL + HPP-1 (400 nM) | 106.62 | 0.82 |
| RANKL + HPP-2 (400 nM) | 73.81 | 2.71 |
| RANKL + HPP-3 (400 nM) | 59.23 | 2.66 |
| RANKL + HPP-4 (400 nM) | 59.76 | 3.58 |

TABLE 3C
Mmp9

| | Fold Change from | |
|---|---|---|
| Cell Treatment | Control (Ave) | St. Dev. |
| Control | 1.00 | 0.08 |
| RANKL | 42.90 | 3.49 |
| RANKL + HPP-1 (400 nM) | 39.73 | 0.44 |
| RANKL + HPP-2 (400 nM) | 24.02 | 0.59 |
| RANKL + HPP-3 (400 nM) | 23.89 | 2.35 |
| RANKL + HPP-4 (400 nM) | 20.21 | 1.43 |

TABLE 3D
Cathepsin K

| | Fold Change from | |
|---|---|---|
| Cell Treatment | Control (Ave) | St. Dev. |
| Control | 1.00 | 0.03 |
| RANKL | 7.83 | 0.52 |
| RANKL + HPP-1 (400 nM) | 5.96 | 0.21 |
| RANKL + HPP-2 (400 nM) | 4.42 | 0.23 |
| RANKL + HPP-3 (400 nM) | 3.31 | 0.03 |
| RANKL + HPP-4 (400 nM) | 2.99 | 0.14 |

TABLE 3E
Dcstamp

| | Fold Change from | |
|---|---|---|
| Cell Treatment | Control (Ave) | St. Dev. |
| Control | 1.00 | 0.15 |
| RANKL | 4.32 | 0.18 |
| RANKL + HPP-1 (400 nM) | 3.33 | 0.34 |
| RANKL + HPP-2 (400 nM) | 2.55 | 0.16 |
| RANKL + HPP-3 (400 nM) | 2.14 | 0.19 |
| RANKL + HPP-4 (400 nM) | 2.39 | 0.15 |

TABLE 3F
Oscar

| | Fold Change from | |
|---|---|---|
| Cell Treatment | Control (Ave) | St. Dev. |
| Control | 1.00 | 0.18 |
| RANKL | 23.88 | 5.96 |
| RANKL + HPP-1 (400 nM) | 20.22 | 3.78 |
| RANKL + HPP-2 (400 nM) | 10.03 | 1.28 |
| RANKL + HPP-3 (400 nM) | 6.45 | 0.65 |
| RANKL + HPP-4 (400 nM) | 8.18 | 0.83 |

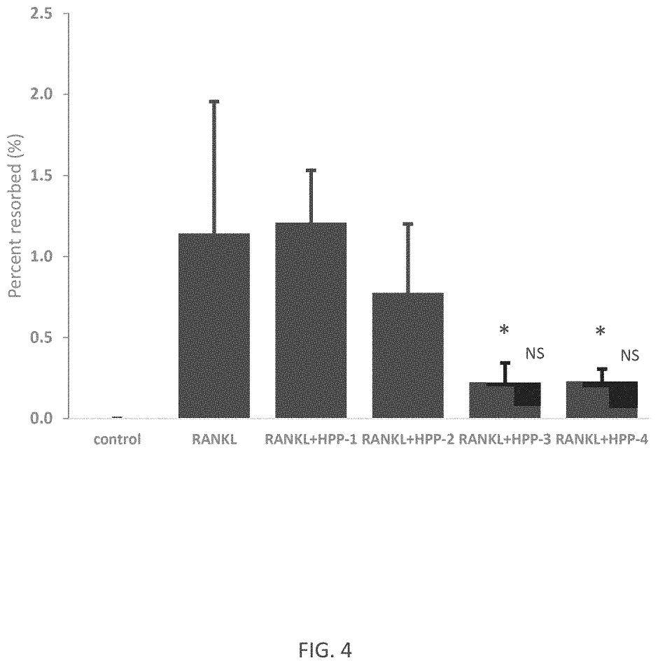

Next, resorption activity was examined using the bone resorption assay plate. RANKL stimulation of RAW264.7 cells induced numerous resorption areas on the substrate, and HPP-3 and HPP-4 reduced the resorption areas such that there was no significant difference from control. These results are summarized in Table 4 below and in FIG. 4.

TABLE 4
Bone Resorption Assay

| | Ave of Resorbed Area | |
|---|---|---|
| Cell Treatment | per Filed (%) | St. Dev. |
| Control | 0.00 | 0.00 |
| RANKL | 1.14 | 0.81 |
| RANKL + HPP-1 (400 nM) | 1.21 | 0.32 |
| RANKL + HPP-2 (400 nM) | 0.78 | 0.43 |
| RANKL + HPP-3 (400 nM) | 0.22 | 0.12 |
| RANKL + HPP-4 (400 nM) | 0.23 | 0.08 |

These results of the real-time RT-PCR analysis and the bone resorption assay suggested that HPP-4 inhibited not only osteoclastogenesis, but also osteoclast activity.

4. HPP-4 Increased Nuclear Nrf2

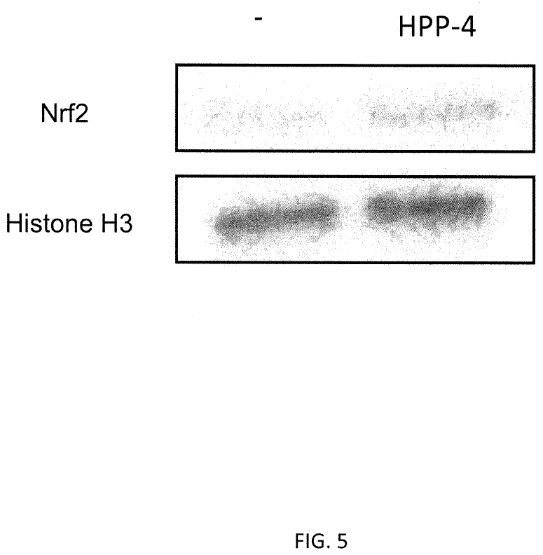

The molecular inhibitory mechanism of HPP-4 on osteoclastic signaling was examined. Western blotting using nuclear extracts of RAW264.7 cells demonstrated that stimulation of RAW264.7 cells with HPP-4 led to nuclear translocation of Nrf2 (See FIG. 5).

5. HPP-4 Induced the Expression of Antioxidant Enzymes

To further examine the effect of HPP-4 on the cellular antioxidant response, the expression of antioxidant enzymes such as NQO1 and HMOX1. The expressions of these two antioxidant enzymes were increased by HPP-4 (400 nM) in RAW264.7 cells (p<0.05) relative to control. This indicates that HPP-4 substantially induces an antioxidant response in RAW264.7 cells. (See Tables 5A and 5B below).

TABLE 5A
NQO1

| | Fold Change from | |
|---|---|---|
| Cell Treatment | Control (Ave) | St. Dev. |
| Control | 1.00 | 0.10 |
| HPP-4 (400 nM) | 2.67 | 0.32 |

TABLE 5B
HMOX1

| | Fold Change from | |
|---|---|---|
| Cell Treatment | Control (Ave) | St. Dev. |
| Control | 1.00 | 0.06 |
| HPP-4 (400 nM) | 1.62 | 0.01 |

6. HPP-4 Attenuated RANKL-Mediated Intracellular ROS

Flow cytometry was used to investigate whether HPP-4 could interfere with RANKL-triggered intracellular ROS production in RAW264.7 cells. Treatment of RAW264.7 cells with RANKL increased intracellular production of superoxide, as detected using BES-So-AM. Treatment with HPP-4 inhibited this RANKL-mediated increased in intracellular ROS, indicating that HPP-4 attenuated RANKL signaling by decreasing superoxide production.

7. Local HPP-4 Injection Ameliorated RANKL-Dependent Bone Destruction in Mice

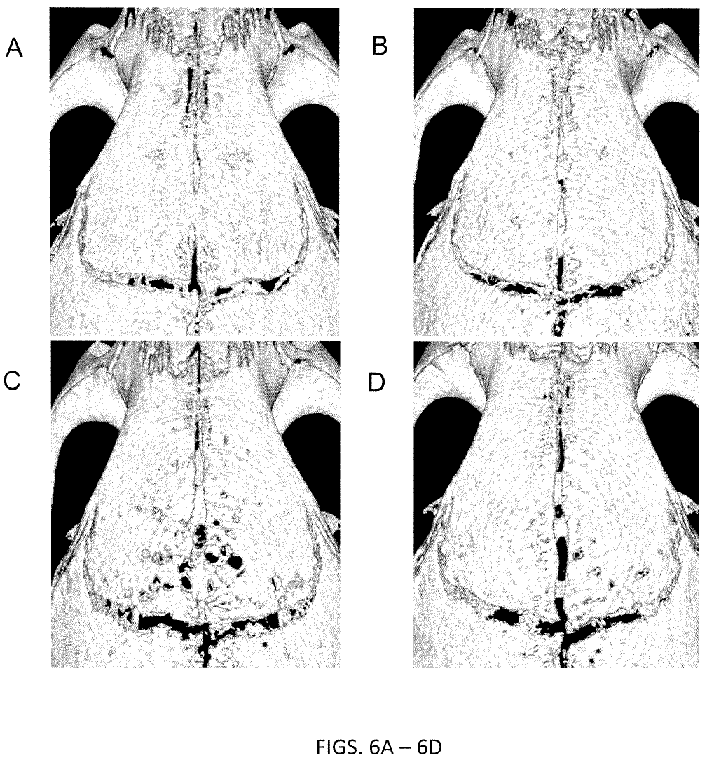

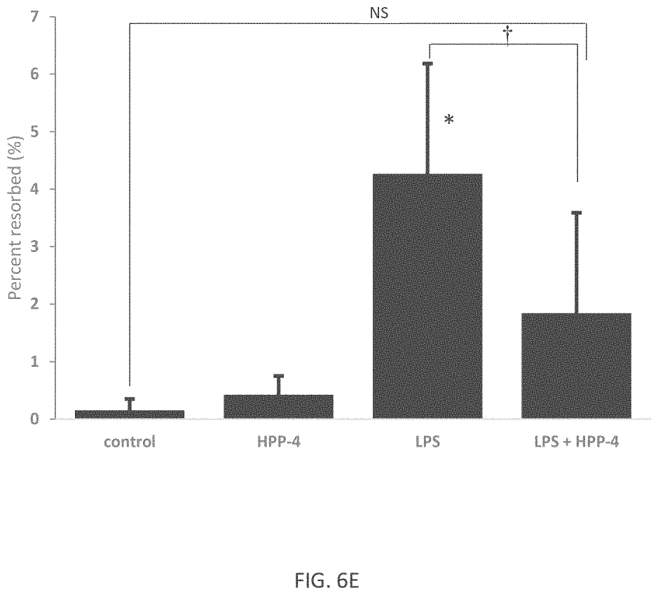

Local injection of HPP-4 was examined to determine whether HPP-4 could ameliorate LPS-mediated bone destruction in mice calvaria. Repeated LPS injection induced bone destruction in mice compared with the control group (Compare FIGS. 6A and 6C). Local injection of HPP-4 ameliorated LPS-mediated bone destruction as demonstrated by μCT imaging of resorbed areas in calvaria (Compare FIGS. 6C and 6D). Resorbed areas were measured and found that HPP-4 significantly inhibited LPS-mediated bone destruction. These results suggested that HPP-4 is a potential inhibitor against bone destruction. Results are summarized in Table 6 below and in FIG. 6E.

TABLE 6
In Vivo Bone Destruction

| | Percent Resorbed | |
|---|---|---|
| Cell Treatment | (%) | St. Dev. |
| Control | 0.15 | 0.20 |
| HPP-4 (400 nM) | 0.42 | 0.33 |
| LPS | 4.27 | 1.93 |
| LPS + HPP-4 (400 nM) | 1.84 | 1.75 |

1. A method of inhibiting osteoclastogenesis or inhibiting osteoclast activity comprising:
administering to a subject a compound of Formula (I) or a pharmaceutically acceptable salt thereof,
wherein
a compound of Formula (I) has the structure shown below 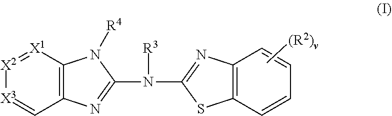
wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h} R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or 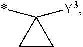
where Y³ is cyclopropyl, —CF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{z};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
a) -halogen,
b) —C₁₋₆ alkyl,
c) —C₃₋₁₀ cycloalkyl,
d) -heterocyclyl,
e) -cyano,
f) —CF₃,
g) —OCF₃,
h) —O—R^{d},
i) —S(O)_{w}—R^{d},
j) —S(O)₂O—R^{d},
k) —NR^{d}R^{e},
l) —C(O)—R^{d},
m) —C(O)—O—R^{d},
n) —OC(O)—R^{d},
o) —C(O)NR^{d}R^{e},
p) —C(O)-heterocyclyl,
q) —NR^{d}C(O)R^{e},
r) —OC(O)NR^{d} R^{e},
s) —NR^{d} C(O)OR^{d}, or
t) —NR^{d} C(O)NR^{d} R^{e},
where the alkyl, cycloalkyl, and heterocyclyl groups are optionally substituted one or more times with substituents independently selected from R^{y};
R^{b} is
a) -halogen,
b) —C₁₋₆ alkyl,
c) —C₃₋₁₀ cycloalkyl,
d) -heterocyclyl,
e) -phenyl,
f) -heteroaryl,
g) -cyano,
h) —CF₃,
i) —OCF₃,
j) —O—R^{f},
k) —S(O)_{w}—R^{f},
l) —S(O)₂O—R^{f},
m) —NR^{f}R^{g},
n) —C(O)—R^{f},
o) —C(O)—O—R^{f},
p) —OC(O)—R^{f},
q) —C(O)NR^{f}R^{g},
r) —C(O)-heterocyclyl,
s) —NR^{f} C(O)R^{g},
t) —OC(O)NR^{f} R^{g},
u) —NR^{f} C(O)OR^{f}, or
v) —NR^{f} C(O)NR^{f} R^{g},
where the alkyl, cycloalkyl, heterocyclyl, phenyl, and heteroaryl groups are optionally substituted one or more times with substituents independently selected from R^{z};
R^{c} is
a) -halogen,
b) —C₁₋₆ alkyl,
c) —C₃₋₁₀ cycloalkyl,
d) -heterocyclyl,
e) -cyano,
f) —CFs,
g) —OCF₃,
h) —O—R^{h},
i) —S(O)_{w}—R^{h},
j) —S(O)₂O—R^{h},
k) —NR^{h}R^{k},
l) —C(O)—R^{h},
m) —C(O)—O—R^{h},
n) —OC(O)—R^{h},
o) —C(O)NR^{h} R^{k},
p) —C(O)-heterocyclyl,
q) —NR^{h} C(O)R^{k},
r) —OC(O)NR^{h} R^{k},
s) —NR^{h} C(O)OR^{k},
t) —NR^{h} C(O)NR^{h} R^{k},
u) —NR^{h} S(O)_{w}R^{k},
v) -phenyl,
w) -heteroaryl, or
x) —O—(C₁₋₄ alkylene)-O—(C₁₋₄ alkylene)-N(R^{h})C(O)—OR^{k},
where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
a) -halogen,
b) —NH₂,
c) -cyano,
d) -carboxy,
e) -hydroxy,
f) -thiol,
g) —CF₃,
h) —OCF₃,
i) —C(O)—NH₂,
j) —S(O)₂—NH₂,
k) oxo,
l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
z) —S(O)₂—N(C₁₋₆ alkyl)₂, optionally substituted one or more times with substituents selected independently from the group consisting of halogen, —OH, —O—C₁₋₆ alkyl, —NH₂, —NH-Cue alkyl, and —N(C₁₋₆ alkyl)₂,
aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
a) —R^{y}
b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
R^{z} is
a) —R^{y}
b) -phenyl,
c) -heteroaryl;
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
v is an integer from 0 to 4, and
w is an integer from 0 to 2.

2. The method of claim 1, wherein the method further comprises the step of determining whether a subject is at risk for or has a bone related disease by
obtaining or having obtained a biological sample from the subject and
performing or having performed a bodily fluid test on the biological sample to determine if the subject has biomarkers for a bone related disease.

3. The method of claim 1, wherein the method further comprises the step of
determining whether a subject is at risk for or has a bone related disease by
performing or having performed a first skeletal survey on an area of the subject's skeleton to determine if the subject has a bone density level associated with a bone related disease.

4. The method of claim 2, wherein the method further comprises the step of
obtaining or having obtained biological samples over a period from the subject,
performing or having performed a bodily fluid test on the biological samples to determine whether the level of one or more biochemical resorptive markers are increasing or decreasing over the period, and
if the level of one or more biochemical resorptive markers are increasing or are not decreasing then administering a greater dose of a compound of Formula (I) or a pharmaceutically acceptable salt thereof.

5. The method of claim 3, wherein the method further comprises the step of
performing or having performed a second skeletal survey to determine whether the subject's bone density has changed from the first skeletal survey, and
if the subject's bone density has decreased from the first to the second skeletal survey then administering a greater dose and/or a more frequent dose of a compound of Formula (I) or a pharmaceutically acceptable salt thereof.

6. The method of claim 1, wherein the compound of Formula (I) or a pharmaceutically acceptable salt thereof is selected from the group consisting of:
1-Methyl-2-(6-trifluoromethoxy-benzothiazol-2-ylamino)-1H-benzoimidazole-5-carboxylic acid ((S)-2-hydroxy-propyl)-amide;
3-Methyl-2-(6-trifluoromethoxy-benzothiazol-2-ylamino)-3H-imidazo[4,5-b]pyridine-6-carboxylic acid (2-methoxy-ethyl)-amide,
1-Methyl-2-(6-trifluoromethyl-benzothiazol-2-ylamino)-1H-benzimidazole-5-carboxylic acid dimethylcarbamoylmethyl-amide; and
1-Methyl-2-(6-trifluoromethyl-benzothiazol-2-ylamino)-1H-benzoimidazole-5-carboxylic acid [2-(2-hydroxy-ethoxy)-ethyl]-amide,
or a pharmaceutically acceptable salt thereof.

7. A method of treating periodontitis or gingivitis comprising
administering to a subject a compound of Formula (I) or a pharmaceutically acceptable salt thereof,
wherein
a compound of Formula (I) has the structure shown below 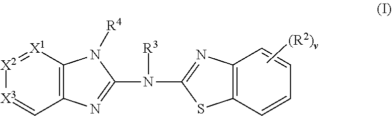
wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h} R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or 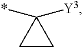
where Y³ is cyclopropyl, —CF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{z};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
a) -halogen,
b) —C₁₋₆ alkyl,
c) —C₃₋₁₀ cycloalkyl,
d) -heterocyclyl,
e) -cyano,
f) —CF₃,
g) —OCF₃,
h) —O—R^{d},
i) —S(O)_{w}—R^{d},
j) —S(O)₂O—R^{d},
k) —NR^{d}R^{e},
l) —C(O)—R^{d},
m) —C(O)—O—R^{d},
n) —OC(O)—R^{d},
o) —C(O)NR^{d}R^{e},
p) —C(O)-heterocyclyl,
q) —NR^{d}C(O)R^{e},
r) —OC(O)NR^{d} R^{e},
s) —NR^{d} C(O)OR^{d}, or
t) —NR^{d} C(O)NR^{d} R^{e},
where the alkyl, cycloalkyl, and heterocyclyl groups are optionally substituted one or more times with substituents independently selected from R^{y};
R^{b} is
a) -halogen,
b) —C₁₋₆ alkyl,
c) —C₃₋₁₀ cycloalkyl,
d) -heterocyclyl,
e) -phenyl,
f) -heteroaryl,
g) -cyano,
h) —CF₃,
i) —OCF₃,
j) —O—R^{f},
k) —S(O)_{w}—R^{f},
l) —S(O)₂O—R^{f},
m) —NR^{f}R^{g},
n) —C(O)—R^{f},
o) —C(O)—O—R^{f},
p) —OC(O)—R^{f},
q) —C(O)NR^{f}R^{g},
r) —C(O)-heterocyclyl,
s) —NR^{f} C(O)R^{g},
t) —OC(O)NR^{f} R^{g},
u) —NR^{f} C(O)OR^{f}, or
v) —NR^{f} C(O)NR^{f} R^{g},
where the alkyl, cycloalkyl, heterocyclyl, phenyl, and heteroaryl groups are optionally substituted one or more times with substituents independently selected from R^{z};
R^{c} is
a) -halogen,
b) —C₁₋₆ alkyl,
c) —C₃₋₁₀ cycloalkyl,
d) -heterocyclyl,
e) -cyano,
f) —CFs,
g) —OCF₃,
h) —O—R^{h},
i) —S(O)_{w}—R^{h},
j) —S(O)₂O—R^{h},
k) —NR^{h}R^{k},
l) —C(O)—R^{h},
m) —C(O)—O—R^{h},
n) —OC(O)—R^{h},
o) —C(O)NR^{h} R^{k},
p) —C(O)-heterocyclyl,
q) —NR^{h} C(O)R^{k},
r) —OC(O)NR^{h} R^{k},
s) —NR^{h} C(O)OR^{k},
t) —NR^{h} C(O)NR^{h} R^{k},
u) —NR^{h} S(O)_{w}R^{k},
v) -phenyl,
w) -heteroaryl, or
x) —O—(C₁₋₄ alkylene)-O—(C₁₋₄ alkylene)-N(R^{h})C(O)—OR^{k},
where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
a) -halogen,
b) —NH₂,
c) -cyano,
d) -carboxy,
e) -hydroxy,
f) -thiol,
g) —CF₃,
h) —OCF₃,
i) —C(O)—NH₂,
j) —S(O)₂—NH₂,
k) oxo,
l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
z) —S(O)₂—N(C₁₋₆ alkyl)₂, optionally substituted one or more times with substituents selected independently from the group consisting of halogen, —OH, —O—C₁₋₆ alkyl, —NH₂, —NH-Cue alkyl, and —N(C₁₋₆ alkyl)₂,
aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
a) —R^{y}
b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
R^{z} is
a) —R^{y}
b) -phenyl,
c) -heteroaryl;
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
v is an integer from 0 to 4, and
w is an integer from 0 to 2.

8. The method of claim 7, wherein a compound of Formula (I) or a pharmaceutically acceptable salt thereof is administered in an amount sufficient to inhibit osteoclastogenesis or osteoclast activity in the subject.

9. The method of claim 8, wherein the method further comprises the step of
determining whether a subject is at risk for or has a bone related disease by
obtaining or having obtained a biological sample from the subject and
performing or having performed a bodily fluid test on the biological sample to determine if the subject has biomarkers for a bone related disease.

10. The method of claim 8, wherein the method fun her comprises the step of
determining whether a subject is at risk for or has a bone related disease by
performing or having performed a first skeletal survey on an area of the subject s skeleton to determine if the subject has a bone density level associated with a bone related disease.

11. The method of claim 9, wherein the method further comprises the step of
obtaining or having obtained biological samples over a period from the subject,
performing or having performed a bodily fluid test on the biological samples to determine whether the level of one or more biochemical resorptive markers are increasing or decreasing over the period, and
if the level of one or more biochemical resorptive market s are increasing or are not decreasing then administering a greater dose of a compound of Formula (I) or a pharmaceutically acceptable salt thereof.

12. The method of claim 10, wherein the method further comprises the step of
performing or having performed a second skeletal survey to determine whether the subject's bone density has changed from the first skeletal survey, and
if the subject's bone density has decreased from the first to the second skeletal survey then administering a greater dose and/or a mote frequent dose of a compound of Formula (I) or a pharmaceutically acceptable salt thereof.

13. The method of claim 7, wherein the compound of Formula (I) or a pharmaceutically acceptable salt thereof is selected from the group consisting of
1-Methyl-2′-(6-trifluoromethoxy-benzothiazol-2-ylamino)-1H-benzoimidazole-5-carboxylic acid ((S)-2-hydroxy-propyl)-amide,
3-Methyl-2-(6-trifluoromethoxy-benzothiazol-2-ylamino)-3H-imidazo[4,5-b]pyridine-6-carboxylic acid (2-methoxy-ethyl)-amide,
1-Methyl-2-(6-trifluoromethyl-benzothiazol-2-ylamino)-1H-benzimidazole-5-carboxylic acid dimethylcarbamoylmethyl-amide; and
1-Methyl-2-(6-trifluoromethyl-benzothiazol-2-ylamino)-1H-benzimidazole-5-carboxylic acid [2-f2-hydroxy-ethoxy)-ethyl]-amide;
or a pharmaceutically acceptable salt thereof.

14. A method of inhibiting bone destruction, inhibiting bone loss, of inhibiting the rate of reduction of bone density, of maintaining bone density, or of increasing bone density comprising
administering to a subject a compound of Formula (I) or a pharmaceutically acceptable salt thereof,
wherein
a compound of Formula (I) has the structure shown below 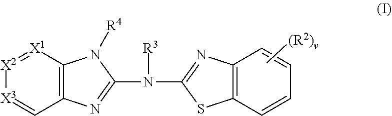
wherein
X¹ is ═N— or ═CH—;
X² is ═C(R¹)— and X³ is ═C(-L-G)-; or X² is ═C(-L-G)- and X³ is ═C(R¹)—;
G is hydrogen, —C₁₋₈ alkyl, —C₃₋₁₀ cycloalkyl, —C₁₋₆ alkylene-C₃₋₁₀ cycloaklyl, heterocyclyl, —C₁₋₆ alkylene-C₃₋₁₀ heterocyclyl, phenyl, heteroaryl, or NR^{h} R^{k}, where the alkyl, alkylene, cycloalkyl, heterocyclyl, phenyl, and heteroaryl groups are optionally substituted one or more times with substituents independently selected from R^{c}; or G is —CH₂Y³, —CH₂CH₂Y³, —CH₂CH₂CH₂Y³, —CH(CH₃)CH₂Y³, —CH₂CH(Y³)CH₃, —CH(Y³)CH₃, —CH₂C(Y³)(CH₃)₂, —C(Y³)(CH₃)₂, or 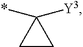
where Y³ is cyclopropyl, —CF₃, —OCH₃, —OCH₂CH₃, —F, —Cl, —OH, —O(CH₂)₂—OH, —O(CH₂)₂—F, —SCH₃, —S(O)₂—CH₃, —SCH₂CH₃, —S(O)₂CH₂CH₃, —NH—CH₃, —NH—CH₂CH₃, —N(CH₃)₂, tetrahydropyran-4-yl, tetrahydrofuran-2-yl, morpholin-2-yl, morpholin-4-yl, piperidin-1-yl, 4-hydroxy-piperidin-1-yl, 3-hydroxy-piperidin-1-yl, —NH—C(O)—CH₃, —NH—C(O)—CH₂CH₃, tetrahydrofuran-2-yl-methyloxy, or —C(O)—Y⁴, where Y⁴ is —OH, —OCH₃, —OCH₂CH₃, —OC(CH₃)₃, —NH₂, —NH—CH₃, —NH—CH₂CH₃, —N(CH₃)₂, —N(CH₂CH₃)₂, morpholin-4-yl, 4-methyl-piperazin-1-yl, pyrrolidin-1-yl, or piperazin-1-yl;
L is —CH₂—C(O)N(R⁶)—, —C(O)N(R⁶)—, —C(O)—O—, —SO₂—, —C(O)—, heteroarylene optionally substituted one or more times with substituents independently selected from R^{x}, or heterocyclylene optionally substituted one or more times with substituents independently selected from R^{x}; or the group -L-G is -cyano;
R¹ is hydrogen, R^{a}, phenyl, or heteroaryl, where the phenyl and heteroaryl groups are optionally substituted one or more times with substituents independently selected from R^{x};
R² is R^{b};
R³ is hydrogen, —C₁₋₆ alkyl, or —C₁₋₆ alkylene-C₃₋₁₀ cycloaklyl, where the alkyl, alkylene, and cycloalkyl groups are optionally substituted one or more times with substituents independently selected from R^{z};
R⁴ is —C₁₋₆ alkyl or —C₁₋₆ alkylene-C₃₋₁₀ cycloaklyl, where the alkyl, alkylene, and cycloalkyl groups are optionally substituted one or more times with substituents independently selected from R^{y};
R⁶ is hydrogen, —C₁₋₆ alkyl, —C₁₋₆ alkylene-C₃₋₁₀ cycloaklyl, where the alkyl, alkylene, and cycloalkyl groups are optionally substituted one or more times with substituents independently selected from R^{x};
R^{a} is
a) -halogen,
b) —C₁₋₆ alkyl,
c) —C₃₋₁₀ cycloalkyl,
d) -heterocyclyl,
e) -cyano,
f) —CF₃,
g) —OCF₃,
h) —O—R^{d},
i) —S(O)_{w}—R^{d},
j) —S(O)₂O—R^{d},
k) —NR^{d}R^{e},
l) —C(O)—R^{d},
m) —C(O)—O—R^{d},
n) —OC(O)—R^{d},
o) —C(O)NR^{d}R^{e},
p) —C(O)-heterocyclyl,
q) —NR^{d}C(O)R^{e},
r) —OC(O)NR^{d} R^{e},
s) —NR^{d} C(O)OR^{d}, or
t) —NR^{d} C(O)NR^{d} R^{e},
where the alkyl, cycloalkyl, and heterocyclyl groups are optionally substituted one or more times with substituents independently selected from R^{y};
R^{b} is
a) -halogen,
b) —C₁₋₆ alkyl,
c) —C₃₋₁₀ cycloalkyl,
d) -heterocyclyl,
e) -phenyl,
f) -heteroaryl,
g) -cyano,
h) —CF₃,
i) —OCF₃,
j) —O—R^{f},
k) —S(O)_{w}—R^{f},
l) —S(O)₂O—R^{f},
m) —NR^{f}R^{g},
n) —C(O)—R^{f},
o) —C(O)—O—R^{f},
p) —OC(O)—R^{f},
q) —C(O)NR^{f}R^{g},
r) —C(O)-heterocyclyl,
s) —NR^{f} C(O)R^{g},
t) —OC(O)NR^{f} R^{g},
u) —NR^{f} C(O)OR^{f}, or
v) —NR^{f} C(O)NR^{f} R^{g},
where the alkyl, cycloalkyl, heterocyclyl, phenyl, and heteroaryl groups are optionally substituted one or more times with substituents independently selected from R^{z};
R^{c} is
a) -halogen,
b) —C₁₋₆ alkyl,
c) —C₃₋₁₀ cycloalkyl,
d) -heterocyclyl,
e) -cyano,
f) —CFs,
g) —OCF₃,
h) —O—R^{h},
i) —S(O)_{w}—R^{h},
j) —S(O)₂O—R^{h},
k) —NR^{h}R^{k},
l) —C(O)—R^{h},
m) —C(O)—O—R^{h},
n) —OC(O)—R^{h},
o) —C(O)NR^{h} R^{k},
p) —C(O)-heterocyclyl,
q) —NR^{h} C(O)R^{k},
r) —OC(O)NR^{h} R^{k},
s) —NR^{h} C(O)OR^{k},
t) —NR^{h} C(O)NR^{h} R^{k},
u) —NR^{h} S(O)_{w}R^{k},
v) -phenyl,
w) -heteroaryl, or
x) —O—(C₁₋₄ alkylene)-O—(C₁₋₄ alkylene)-N(R^{h})C(O)—OR^{k},
where the alkylene, alkyl, cycloalkyl, heterocyclyl, phenyl, and heteroaryl groups are optionally substituted one or more times with substituents independently selected from R^{x};
R^{d} and R^{e} are independently hydrogen, C₁₋₆ alkyl, or C₃₋₁₀ cycloalkyl, where the alkyl and cycloalkyl groups are optionally substituted one or more times with substituents independently selected from R^{y}; or, if R^{d} and R^{e} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{y};
R^{f} and R^{g} are independently hydrogen, C₁₋₆ alkyl, C₃₋₁₀ cycloalkyl, phenyl, or heteroaryl, where the alkyl, cycloalkyl, phenyl, and heteroaryl groups are optionally substituted one or more times with substituents independently selected from R^{z}; or, if R^{f} and R^{g} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{z};
R^{h} and R^{k} are independently hydrogen, C₁₋₆ alkyl, C₃₋₁₀ cycloalkyl, heterocyclyl, phenyl, or heteroaryl, where the alkyl, cycloalkyl, heterocyclyl, phenyl, and heteroaryl groups are optionally substituted one or more times with substituents independently selected from R^{x}; or, if R^{h} and R^{k} are both attached to the same nitrogen atom, together with that nitrogen atom may optionally form a heterocyclic ring selected from the group consisting of azetidino, pyrrolidino, pyrazolidino, imidazolidino, oxazolidino, isoxazolidino, thiazolidino, isothiazolidino, piperidino, piperazino, morpholino, thiomorpholino, and azepano, where each ring is optionally substituted one or more times with substituents independently selected from R^{x};
R^{y} is
a) -halogen,
b) —NH₂,
c) -cyano,
d) -carboxy,
e) -hydroxy,
f) -thiol,
g) —CF₃,
h) —OCF₃,
i) —C(O)—NH₂,
j) —S(O)₂—NH₂,
k) oxo,
l) —C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
m) -heterocyclyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
n) —C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
o) —O—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
p) —O—C₃₋₁₀ cycloalkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
q) —NH—C₁₋₆ alkyl optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
r) —N(C₁₋₆ alkyl)₂ optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
s) —C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
t) —C(O)—O—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
u) —S—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
v) —S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
w) —C(O)—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
x) —C(O)—N(C₁₋₆ alkyl)₂, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
y) —S(O)₂—NH—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
z) —S(O)₂—N(C₁₋₆ alkyl)₂, optionally substituted one or more times with substituents selected independently from the group consisting of halogen, —OH, —O—C₁₋₆ alkyl, —NH₂, —NH-Cue alkyl, and —N(C₁₋₆ alkyl)₂,
aa) —NH—C(O)—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂, or
bb) —NH—S(O)₂—C₁₋₆ alkyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂;
R^{x} is
a) —R^{y}
b) -phenyl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
c) -heteroaryl, optionally substituted one or more times with substituents selected independently from the group consisting of halogen, —OH, —O—C₁₋₆ alkyl, —NH₂, —NH—C₁₋₆ alkyl, and —N(C₁₋₆ alkyl)₂,
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
R^{z} is
a) —R^{y}
b) -phenyl,
c) -heteroaryl;
d) —O-phenyl,
e) —O-heteroaryl,
f) —C(O)-phenyl,
g) —C(O)-heteroaryl,
h) —C(O)—O-phenyl, or
i) —C(O)—O-heteroaryl;
v is an integer from 0 to 4, and
w is an integer from 0 to 2.

15. The method of claim 14, wherein the amount of a compound of Formula (I) or a pharmaceutically acceptable salt thereof administered is sufficient to inhibit osteoclastogenesis or osteoclast activity in the subject.

16. The method of claim 15, wherein that subject has been diagnosed with periodontitis, gingivitis, osteoporosis, osteoarthritis, or rheumatoid arthritis.

17. The method of claim 15, wherein the method further comprises the step of
determining whether a subject is at risk for or has a bone related disease by
obtaining or having obtained a biological sample from the subject and
performing or having performed a bodily fluid test on the biological sample to determine if the subject has biomarkers for a bone related disease.

18. The method claim 15, wherein the method further comprises the step of
determining whether a subject is at risk for or has a bone related disease by
performing or having performed a first skeletal survey on an area of the subject's skeleton to determine if the subject has a bone density level associated with a bone related disease.

19. The method of claim 17, wherein the method further comprises the step of
obtaining or having obtained biological samples over a period from the subject,
performing or having performed a bodily fluid test on the biological samples to determine whether the level of one or more biochemical resorptive markers are increasing or decreasing over the period, and
if the level of one or more biochemical resorptive markers are increasing or are not decreasing then administering a greater dose of a compound of Formula (I) or a pharmaceutically acceptable salt thereof.

20. The method of claim 18, wherein the method further comprises the step of
performing or having performed a second skeletal survey to determine whether the subject's bone density has changed from the first skeletal survey, and
if the subject's bone density has decreased from the first to the second skeletal survey-then administering a greater dose and/or a more frequent dose of a compound of Formula (I) or a pharmaceutically acceptable salt thereof.

21. The method of claim 14, wherein the compound of Formula (I) or a pharmaceutically acceptable salt thereof is selected from the group consisting of
1-Methyl-2-(6-trifluoromethoxy-benzothiazol-2-ylamino)-1H-benzoimidazole-5-carboxylic acid ((S)-2-hydroxy-propyl)-amide-;
3-Methyl-2-(6-trifluoromethoxy-benzothiazol-2-ylamino)-3H-imidazo[4,5-b]pyridine-6-carboxylic acid (2-methoxy-ethyl)-amide;
1-Methyl-2-(6-trifluoromethyl-benzothiazol-2-ylamino)-1H-benzimidazole-5-carboxylic acid dimethylcarbamoylmethyl-amide; and
1-Methyl-2-(6-trifluoromethyl-benzothiazol-2-ylamino)-1H-benzoimidazole-5-carboxylic acid [2-(2-hydroxy-ethoxy)-ethyl]-amide;
or a pharmaceutically acceptable salt thereof.
